(19) United States
(12) Patent Application Publication
(10) Pub. No.: US 2021/0341378 A1
(43) Pub. Date: Nov. 4, 2021

(54) HIGH-CONTENT IMAGING OF MICROFLUIDIC DEVICES

(71) Applicant: EMULATE, Inc., Boston, MA (US)

(72) Inventors: Kyung-Jin Jang, Boston, MA (US); Daniel Levner, Brookline, MA (US); Konstantia-Roumvini Kodella, Cambridge, MA (US); Jonathan Rubins, Cambridge, MA (US); Debora Barreiros Petropolis, Cambridge, MA (US); Samatha Peel, Cambridge (GB); Adam M. Corrigan, Cambridge (GB); Beate Ehrardt, 1 Francis Crick Ave (GB); Pedro Pinto, Cambridge (GB); Dominic Williams, Cambridge (GB); Matt Boeckeler, Waltham, MA (US); Alison J. Foster, Cambridge (GB); Geraldine Hamilton, Boston, MA (US); Lorna Ewart Ewart, Cambridge (GB)

(21) Appl. No.: 17/375,237

(22) Filed: Jul. 14, 2021

Related U.S. Application Data
(63) Continuation of application No. PCT/US2020/014590, filed on Jan. 22, 2020.
(60) Provisional application No. 62/916,483, filed on Oct. 17, 2019.
(60) Provisional application No. 62/834,004, filed on Apr. 15, 2019.
(60) Provisional application No. 62/828,849, filed on Apr. 3, 2019.
(60) Provisional application No. 62/795,345, filed on Jan. 22, 2019.

(51) Int. Cl. G01N 15/14; G01N 33/50; G02B 21/00

(52) U.S. Cl. CPC G01N 15/1434; G01N 33/5067; G01N 2015/144; G02B 21/0072; G02B 21/008

(57) ABSTRACT

The present invention is related to high-content microscopy imaging of microfluidic cell culture systems. A method of high-content microfluidic device microscopy is contemplated. along with related statistical analysis and microfluidic device adaptors.

### CROSS-REFERENCE TO RELATED APPLICATIONS

This application claims priority to U.S. Provisional Patent Applications No. 62/795,345 filed Jan. 22, 2019; 62/828,849 filed Apr. 3, 2019; 62/834,004 filed Apr. 15, 2019; and Ser. No. 37/487,696 filed Oct. 17, 2019, herein incorporated by reference in their entireties.

### FIELD OF INVENTION

The present invention is related to high-content microscopy imaging of microfluidic cell culture systems. A method of high-content microfluidic device microscopy is contemplated, along with related statistical analysis and microfluidic device adaptors.

### BACKGROUND

The pharmaceutical industry needs to improve the probability of success of drugs reaching late stage clinical trial. One category of microfluidic devices are micro-engineered systems that aim to recapitulate the organ microenvironment for drug discovery. Microfluidic devices comprising cells of human origin have been adopted for enhancing pre-clinical efficacy and toxicity evaluation and prediction. While capturing cellular phenotype via imaging in response to drug exposure is a useful readout in these models, this application has been limited due to difficulties in imaging the microfluidic devices robustly and at scale.

### SUMMARY OF INVENTION

The present invention is related to high-content microscopy imaging of microfluidic cell culture systems. A method of high-content microfluidic device microscopy is contemplated, along with related statistical analysis and a microfluidic device adaptor conFIG.d to decrease imaging variability.

In the drug discovery process, the ability to accurately detect and predict both efficacy and safety relies on the availability of robust assay models that reproduce, as closely as possible, the effects at both a human volunteer and patient level. The relative simplicity of two-dimensional cell culture models means that findings often do not translate into the clinical phases of drug development. Therefore, complex culture systems (including three-dimensional and co-culture systems) are an area of intense interest because of their potential for improved translatability whilst retaining the control and ease of handling such systems. Microfluidic devices comprising organ cells, variously known as Organ Chips, organs-on-a-chip, tissue chips, organomimetic devices or microphysiological systems, aim to recapitulate the complex physiology and microenvironment of an in vivo organ through spatiotemporal control of tissue architecture and addition of fluid dynamics.

The use of microfluidic devices that more accurately recapitulate organ and disease biology have the potential to impact aspects of drug discovery from target validation, elucidation of mechanism of action, and compound efficacy, through to safety and pharmacokinetic profiling of drugs destined for the clinic. Some examples of types of drugs for treating a disorder or disease include but are not limited to: prophylactic compounds (i.e. delay of onset and/or prevention); compounds for reducing symptoms; compounds for extending lifespan; compounds for preventing onset of symptoms; etc. Nonlimiting examples of compounds are described herein, and include pharmaceutical compounds, chemotherapy compounds, protein therapy, etc.

Such types of drugs may also be used on the context of gene therapy. It is not intended to limit the use of gene therapy, examples include replacing a faulty gene, adding a new gene, such as in an attempt to cure disease; improve your body's ability to fight disease; etc., reducing the expression or turning off a gene that may cause disease, turning on genes for assisting in preventing disease, replacing disease associated nucleic acids with non-disease associated nucleic acids. Some examples of types of diseases that may be cured using gene therapy drugs include but are not limited to those for curing cancer, blindness, immune, and neuronal disorders, to name a few. However, gene therapy treatment has risks including undesirable inflammation, e.g. causing tissue damage, causing organ damage leading to organ failure; targeting healthy cells causing damage that may lead to other illness, diseases and cancer; gene therapy vectors themselves may recover their ability to cause disease and do so; genetic material is inserted into an unintended location with a host cell gene which may lead to tumor formation or other unwanted consequence.

Gene therapy vectors include but are not limited to engineered viruses, plasmids, bacteria, CRISPR constructs, etc., Viral vectors are often used because they express proteins that can recognize certain cells, i.e. cell receptors, and they may physically insert genetic material in the form of nucleic acids into the host cells' genome. Researchers remove disease-causing genes from infectious viruses forming a treatment vector, then add nucleic acid sequences intended for delivery into target/host cells.

Gene therapy virus vectors include but are not limited to adenoviruses, adeno-associated viruses (AAVs), retroviruses, and lentiviruses, Although gene therapy vectors are typically administered via intravenous routes, individual patient-derived cellular gene therapy may be used where cells are removed from the patient, treated, e.g. with gene therapy, compounds, or combinations thereof, and then returned to the patient. Cells obtained from an organisms may be in a liquid, e.g. blood for obtaining white blood cells, lung fluid, plural fluid, lymphatic fluid or as part of a biopsy. A biopsy may be processed for releasing cell clumps and/or single cells for use herein. Examples of cell purification include but are not limited to FACS sorting, affinity columns, density gradient centrifugation, etc. Such patient derived cells may undergo purification steps in order to reduce bystander effects of gene therapy treatments before the treated cells are returned to the patient.

While therapeutic gene therapy is contemplated for many types of disorders and diseases, there is limited accurate preclinical data mainly because plate culture systems lack accurate mimicking of physiological responses and lack accurate mimicking of physiological responses of organs. However, as mentioned herein, microfluidic devices known as Organ Chips have cell type mimicking capabilities and have organ mimicking capabilities that may be used for providing more accurate preclinical data on reactions to gene therapy therapeutics, in particular providing preclinical data for use in evaluating pharmacological compounds for gene therapy. Thus, in some embodiments, cells derived from individuals for use in gene therapy may be tested in a microfluidic device. Thus, in some embodiments, gene therapy treatment responses may be tested in a microfluidic device. In some embodiments, such testing may provide preclinical data for more accurate predictions of cellular responses in patients. Moreover, the use of individual patient derived cells may find use for testing gene therapy treatment responses within a microfluidic device prior to being administered back to the patient.

A role for microfluidic devices will also be towards understanding on- and off-target species differences and how this translates to human physiology. However, there are challenges involved with extracting meaningful and robust data that may be overcome to enable full exploitation of these high-fidelity models. For example, the scale of these systems (approximately 35,000 to 70,000 cells) can in some cases hinder detection of analytes due to the small quantities produced and the small media volumes available for analysis. Imaging via microscopy offers an orthogonal approach for capturing cell phenotype in response to drug exposure. In addition, imaging data and the fact that microfluidic devices provide cellular and molecular level resolution of biology can enable new insights into biological processes and enable mechanistic insights, e.g. mechanism of action of drug for efficacy. However, routine application of imaging has been limited due to difficulties in the ability to image microfluidic devices reproducibly at scale.

Automated microscopes take advantage of the standard layout of multi-well plates to remove the need for manual control of the stage, allowing higher throughput acquisition on large numbers of plates. By contrast, the recent rise of microfluidic systems means that a standard architecture has not yet emerged; indeed, multiple microfluidic device architectures are presently used for various application or modular combination of organs. Furthermore, due to the narrow width of channels on some microfluidic devices, fields of view (FOV) should be placed accurately placed to correctly capture the cells, meaning that field positions often need to be adjusted on a device-by-device basis to avoid introducing imaging biases. Simply placing microfluidic devices in the same location for imaging may not result in consistent imaging, due to the size limitations of microfluidic devices. As such, the combination of an intelligent scanning workflow and an adapter to ensure consistent microfluidic placement on microscope stages could lead to high-content, low-variability microfluidic device imaging. There are a wide range of options for on-the-fly microscope control based on feedback from acquired images, including the MetaXpress journal capability, the Wako Software Suite for Yokogawa systems, through to full microscope control using tools such as MicroManager.

Presented herein, in one embodiment, is an invention consisting of a microfluidic device adapter, a high-content imaging workflow, and a method of statistical analysis for use with microfluidic devices. It is not intended that the present invention be limited by the type of microscope, microfluidic device, or cause for microfluidic device imaging (such as microfluidic device inspection, cellular experiments, bacterial experiments, organism experiments, chemical experiments, diagnostic experiments, for use with personalized medicine, etc.) In one embodiment, where the microfluidic device is seeded with cells, it is not intended that the present invention be limited by the cell type, cell density, etc. The high-content imaging workflow and statistical analysis presented herein may be used to investigate any microfluidic device. The high-content imaging workflow and statistical analysis presented herein are advantageous as they may be implemented across multiple industries that use microfluidic devices and may be used to investigate anything contained within a microfluidic device. The high-content imaging workflow presented herein is also advantageous as it has the potential to vastly increase the efficiency of microfluidic experiments, reduce image variability, improve image quality and remove user bias.

One aspect of the invention presented herein is a microfluidic device adaptor that allows compatibility of microfluidic devices with high throughput, high content microscopes, such as confocal microscopes. The adapter may also exist in a system comprising a microfluidic device adaptor and microfluidic device. There are several embodiments for which the microfluidic device and microfluidic device adaptor interact. The microfluidic device adaptor may comprise alignment features for a one or a plurality of microfluidic devices. In one embodiment, the alignment features comprise cutouts into which the microfluidic devices fit. In another embodiment, the microfluidic device fits into microfluidic device adaptor via a compression fit. In another embodiment, the compression fit is a radial compression fit. In one embodiment, the microfluidic device adaptor interfaces with a microscope. In another embodiment, the microfluidic device interfaces with a microscope stage. In one embodiment, the microfluidic device adaptor comprises interface features to attach to a microscope. In one embodiment, the microfluidic device adaptor comprises interface features to attach to a microscope stage. In one embodiment, the microfluidic device adaptor comprises interface features to attach to a microscope stage consisting of guide rails.

One aspect of the invention presented herein is a high-content imaging workflow that has the capability of reducing acquisition time of microfluidic devices by as much as 95%, reducing imaging variability between microfluidic devices to less than 10%, improving imaging quality and removing user bias. The high-content imaging workflow may be used with any microfluidic device on any microscope that comprises a camera. Many microscopes are envisioned, such as confocal or light microscopes capable of imaging.

In one embodiment of the high-content imaging workflow, a first set of imaging takes places, followed by image analysis, followed by a second set of imaging based off the analysis of the first round of imaging. Image analysis may comprise the mapping out of microfluidic devices or portions of the microfluidic device, in one embodiment to identify a coordinate system within the microfluidic device. In an exemplary embodiment, the high-content imaging workflow presented herein incorporates intelligent scanning to map out the cell chambers within microfluidic devices seeded with cells that are ready for higher magnification imaging. In an exemplary embodiment, the first round of acquisitions may be low-resolution, on which a coordinate system may be identified, the coordinate system may then direct a second round of high-resolution acquisitions. The first set of acquisitions may not need to be high-resolution as it may only be used to identify particular aspects of the microfluidic device; however, depending on the use of the high-content imaging workflow, the first set of acquisitions may be high-resolution. The first and second round of acquisitions may be used with any type of microscopy comprising a camera, such as brightfield or fluorescent. In one embodiment, however, the first round of acquisitions may be brightfield and the second round of acquisitions may be fluorescent. The second round of acquisitions, guided by the coordinate system, may comprise Z stack slices through different layers of the microfluidic device. The process from mapping out the microfluidic device regions to high resolution Z stacking of the interior of each microfluidic device may be fully automated. Furthermore, in one embodiment, it is possible to examine whether specimen within the microfluidic devices show differential responses depending upon their location in a microfluidic device. The setup of this automated workflow has applications to a large variety of microfluidic devices, including multi-cellular microfluidic devices where detailed cellular phenotype (including morphology, proliferation, apoptosis, and mitochondrial function), in response to agents, is desired to be studied.

It is not intended that the invention presented herein be limited by a specific type of coordinate system identified during image analysis. Coordinate systems may be identified using any microfluidic device architecture or geometric criteria. Microfluidic device architecture includes fabricated components of the microfluidic device, such as channels, channel walls, ports, membranes, and other structures. Coordinate systems may also be based off geometric criteria identified within the microfluidic device, such as the shapes, apparent densities, and location of materials contained within the microfluidic device. Geometric criteria identified within the microfluidic device may be based on the presence of cells, organisms, particles, fluids, or anything else placed, cultured or flowed within the microfluidic device. Coordinate systems may be based off anything on or within a microfluidic device.

In one embodiment, microfluidic device architecture may be considered standard across microfluidic devices. In such a case, the high-content imaging workflow may be able to identify that architecture in any microfluidic device comprising said architecture. Standard architecture may comprise anything on or within a microfluidic device. Standard architecture includes microchannels, microchannel walls, access ports, microstructures, etc. In one embodiment, the microfluidic device architecture consists of the locations of surfaces. In one embodiment, the location of surfaces within the microfluidic device is based off the focal height of said surface. In one embodiment, the microfluidic device architecture consists of microfluidic channel walls. In one embodiment, the microfluidic device architecture consists of a membrane. In one embodiment, the microfluidic device architecture consists of pores. In one embodiment, the microfluidic device architecture consists of microchannel access ports. In one embodiment, the microfluidic device architecture consists of microstructures. In one embodiment, the microfluidic device architecture consists of tissue culture anchors, such as for skeletal muscle tissue.

Further, the high-content analysis presented herein is capable of identifying not only microfluidic device architecture, but also geometric criteria within the microfluidic device. Again, any microfluidic device architecture or geometric criteria may be used to set a coordinate system within the images. Geometric criteria include the shapes, apparent densities and location of anything in or on a microfluidic device. Shape factor is a value assigned to the shape of an object, regardless of the object's other dimensions. Shape factors include circularity, eccentricity, solidity, convexity, aspect ratio, elongation, compactness, waviness, and more. As well, geometric criteria used to identify a coordinate system within a microfluidic device may include an objects location in the microfluidic device. In one embodiment, an objects location may be gauged relative to the object's proximity to other objects. In one embodiment, the objects location may be gauged relative to the object's proximity to features of the microfluidic device, such as microfluidic device surfaces, channel walls, ports, microstructures, etc.

Cell culture is emerging as one of the major uses of microfluidic devices. The high-content imaging workflow presented herein lends well for the use of imaging microfluidic devices seeded with cells. The high-content imaging workflow presented is not intended to be limited by the particular purpose for imaging microfluidic devices. In the instance where one is culturing cells in one or more microfluidic devices, there are several reasons to image microfluidic devices and image large numbers of microfluidic devices consecutively. In one embodiment, the microfluidic device is for the use of health diagnostics. In one embodiment, the microfluidic device is used to study chemical reactions. In one embodiment, the microfluidic device houses a small specimen, such as bacteria, worms, etc. In one embodiment, the microfluidic device contains nothing. In one embodiment, the invention presented herein may be used to image and analyze microfluidic device architecture. In one embodiment, the high-content imaging workflow presented herein may be used to image and analyze biological parameters, such as cell morphology, metabolic levels, cellular interactions, proteins levels, lipid levels, acid levels, etc.

In one embodiment, biological parameters may be studied in order to investigate cellular viability or health. Cellular apoptosis, a measurement of cellular death, may be analyzed using the high-content imaging. Cell morphology may include the relative shape and size of cells. Metabolic levels include anything produced by the cells, such as alcohols, amino acids, nucleotides, antioxidants, organic acids, polyols, vitamins, etc. The high-content imaging workflow may be used to image any metabolite imaged in standard cell culture experiments. Cellular interactions to be imaged and analyzed by the high-content imaging protocol include cell junctions, cell signaling, cell immune response, cell coagulation, cellular disease spread, etc. The high-content imaging workflow may be used to image any cellular interaction imaged in standard cell culture experiments.

In one embodiment, the high-content imaging workflow presented herein may be used to image and analyze canaliculus networks, such as bile canalicular networks. A canaliculus is a small passageway or duct. A biocanaliculus is a canaliculus in a biological system such cuniculus within organs, bones, teeth, etc. A bile canalicular network is a system of canaliculus within a biological system. Bile canalicular networks exist in vivo and in vitro, such as within microfluidic devices. In vitro biocanaliculus include cell lined channels in microfluidic devices. In one embodiment, the high-content imaging workflow presented herein may be used to study parameters (size, width, height, length, shape, etc.) of bile canalicular networks and the cells within the bile canalicular networks. In one embodiment, the high-content imaging workflow presented herein may be used to image bile canalicular networks to investigate the health of canaliculi. In one embodiment, the high-content imaging workflow presented herein may be used to investigate whether canaliculi in microfluidic devices are interlocking. Healthy canaliculi tend to interlock.

In one embodiment, cellular interactions between cells contained within bile canalicular channels may be investigated. Protein levels in cellular systems contained within microfluidic devices may also be imaged and analyzed using the high-content imaging protocol presented herein. The high-content imaging workflow may be used to image any protein imaged in standard cellular experiments. Proteins include alpha-smooth muscle actin (α-SMA), vimentin, stabilin-1, hemoglobin, cadherin, ependymin, integrin, NCAM, selectin, CFTR, multidrug resistance-associated protein 2 (MRP2), bile salt export pump (BSEP) protein, monocyte chemoattractant protein-1 (MCP-1), interferon gamma-induced protein 10 (IP-10), etc. Lipid levels and lipid accumulation may also be imaged and analyzed using the high-content imaging workflow. Acid levels within cell cultures in microfluidic device may also be imaged and analyzed using the high-content imaging workflow. The high-content imaging workflow may be used to image any acid imaging during standard cell culture. Acids include bile, such as that in liver tissue, and more.

The high-content imaging workflow presented herein may be especially helpful in imaging cells stained for α-SMA. Cells stained for α-SMA have a non-zero baseline when fluorescently imaging, and therefore it is difficult to distinguish between background and α-SMA related fluorescence. The high-content imaging workflow and related statistical analysis may be able to differentiate between relevant α-SMA fluorescence and background brightness. The same may be said for lipid accumulation imaging. Cells stained for lipid accumulation present a non-zero fluorescence base-line, and are therefore typically difficult to image. The high-content imaging workflow presented herein lends itself to distinguishing between relevant lipid accumulation fluorescence and background brightness.

In one embodiment, the high-content imaging workflow may be used in identifying the presence of nuclear stains on the cells in the microfluidic device. In one embodiment, the high-content imaging workflow may be used in identifying membrane markers between the cells in the microfluidic device. In one embodiment, the membrane markers are tight junction markers. In one embodiment, the tight junction markers are zonula occludens-1 (ZO-1) markers. In one embodiment, the tight junction markers are cadherin markers. In one embodiment, the cadherin markers are epithelial cadherin markers. In one embodiment, the high-content imaging workflow may be used in identifying the presence of a gradient along the length microfluidic device. In one embodiment, the gradients are identified downstream in the microfluidic device channels. In one embodiment, the gradients are identified upstream in the microfluidic device channels. In one embodiment, the gradient is a change in the number of metabolites. In one embodiment, the gradient is an oxygen gradient. In one embodiment, the gradient is a change in the number of nuclei present. Typically conducting imaging of microfluidic devices in order to investigate biological parameters, such as cell morphology, cell junction strength, marker quantification, etc. can take hours per microfluidic device. The high-content imaging workflow presented herein has the capability to image a microfluidic device in as little as five to ten minutes. In a preferred embodiment of the inventions presented herein, the imaging of eight microfluidic devices seeded with cells may be decreased from 16 hours to just 50 minutes, for a time saving of 95%. Coordinate systems may be identified, in one embodiment, based on cells within the microfluidic device. It is not intended that the image analysis and coordinate system identification be limited by the cell type, cell size, cell density, cell age, cell culture length, whether the cell is attached or not to a surface, cell location, etc. Coordinate systems may be identified based on cells of different types, sizes, densities, ages, culture levels, attachment levels, cell location, etc. In one embodiment, cells are cultured on to a surface of the microfluidic device, such as channel walls or membranes, such that they are attached to said surface. In another embodiment, the cells are not attached to any surface, such as channel walls or membranes. The coordinate system may be a targeted cellular microsystem, either two or three dimensional. In one embodiment, the coordinate system is a recapitulated physiological system, such as a tract, vessels, stratified cellular structures, etc.

Geometric criteria used to identify coordinate systems may be based on the location of cells on or within a microfluidic device. In one embodiment, the image analysis identifies the proximity of cells to each other or features of the microfluidic device in order to identify a coordinate system for a second set of acquisitions. The identification of the location of cells within the microfluidic device may be used regardless of cell attachment or not. In one embodiment, it is desired to image cells of a particular location. In this embodiment, the image analysis of the first set of acquisitions detects cells of that particular location, sets a coordinate system about them, and conducts a second set of acquisitions.

Geometric criteria used to identify coordinate systems may be based on cell geometry or shape. Cell shape factors include cell circularity, eccentricity and solidity. Cell shape may be identified in order to then identify a coordinate system for a second set of acquisitions. Cell circularity is the amount the cell is shaped as a circle. A circle has a circularity of one. Circularity is also known as isoperimetric quotient. Cell eccentricity is how much a cell deviates from being circular. The eccentricity of a circle is zero. Oftentimes non-attached cells exhibit more circular shapes, while attached cells exhibit more elongated shapes. Cell solidity, also known as convexity, is the proportion of the cell that fits within a smooth line around the cell. A cell with many protrusions or indentations would have a cell solidity closer to zero than a cell with smooth edges. Other geometric criteria or shape factors that may be used to identify a coordinate system within a microfluidic device are aspect ratio, elongation, compactness, waviness, etc. In one embodiment, it is desired to image cells of a particular shape. In this embodiment, the image analysis of the first set of acquisitions detects cells of that particular shape, sets a coordinate system about them, and conducts a second set of acquisitions. In one embodiment, the image analysis identifies the size of cells in order to then identify a coordinate system. In one embodiment, it is desired to image cells of a particular size. In this embodiment, the image analysis of the first set of acquisitions detects cells of that particular size, sets a coordinate system about them, and conducts a second set of acquisitions.

In one example, cells may be seeded in a microfluidic device in such a way as to recapitulate a biliary canaliculus. In such an embodiment, the coordinate system is a biliary canaliculus. In one embodiment, cell size smaller than 70 μm² and greater than 7 μm², may be used to determine the coordinate system of a biliary canaliculi. In one embodiment, the high-content imaging workflow may detect cell solidity greater than 0.7 in order to determine the coordinate system of a biliary canaliculi. In some cases, jagged, elongated canaliculi are sought during the first round of acquisitions. In some embodiments, the high-content imaging workflow may detect circularity below 0.5 in order to determine the coordinate system of a biliary canaliculi. In some embodiments, the high-content imaging workflow may detect eccentricity greater than 0.8 in order to determine the coordinate system of a biliary canaliculi. Geometric criteria of cells may be compared to known geometric criteria of other objects or object surfaces, including features of a microfluidic device. In one embodiment, the circularity of cells may be compared to the circularity of foreign objects, such as round, synthetic beads. In one embodiment, the size of cells may be compared to the size of foreign objects, such as synthetic beads. Further, geometric criteria, such as cell shape, may be used to gauge cell health. Again, it is not intended that the high-content imaging workflow, or any part of the invention presented herein, be limited by the microfluidic device architecture or geometric criteria chosen for image analysis.

The high-content imaging workflow presented herein may also be used, in some embodiments, to image the effect of an agent or compound on cells, or other biological specimen, within the microfluidic device. Agents to be tested in microfluidic devices include the combination or independent use of pharmaceuticals, cosmetics, food items, chemicals, etc. Further, the high-content imaging workflow presented herein may be used to image cells at a variety of culture levels. It has been shown experimentally, that microfluidic devices to be imaged can be cultured from hours to weeks. In one embodiment, the cells are cultured for less than seven days. In one embodiment, the cells are cultured for more than seven days. In one embodiment, the cells are cultured for more than two weeks.

The second round of acquisition may comprise a Z-stack of images, or a three-dimensional acquisition. In one embodiment, the microfluidic device is seeded with cells. In one embodiment, the cells are located in the channels separated by the membrane. In one embodiment, the three-dimensional acquisition comprises an endothelial cell layer and epithelial cell layer together, separated by the membrane. As an example, the cells may be liver cells. In that embodiment, the liver cells may be hepatocytes and sinusoidal endothelial cells. The liver cells may be of any origin, including, but not limited to, human, dog, and rat. In one embodiment, the hepatocytes and sinusoidal endothelial cells are human hepatocytes and human sinusoidal endothelial cells. In another embodiment, the cells are kidney cells. The second round of acquisition may also comprise individual cells. Again, the microfluidic device is not limited by its design or composition. In one embodiment, the channels of the microfluidic device are coated with a mixture of extracellular matrix. In one embodiment, there is flow within the channels. In one embodiment, there is flow in one channel and not others. In one embodiment, there is no flow within any channels. In one embodiment, flow is introduced into the channels of the microfluidic device in order to exert shear stress on the cells. In one embodiment, flow is introduced to the channels before imaging. In one embodiment, flow is introduced to the channels during imaging. In one embodiment, flow is introduced to the channels before and during imaging.

Another aspect of the invention presented herein is a method to decouple sources of variability in experiments using the high-content imaging workflow and related statistics. In one embodiment, imaging a high number of microfluidic devices using a high-content imaging workflow allows different sources of variability to be decoupled, including microfluidic device, holder row, individual holder effects, etc. by including hyperparameters describing the standard deviation associated with each error source. In one embodiment, the fitting procedure automatically identifies the microfluidic device to microfluidic device, row-to-row and holder-to-holder variability. In one embodiment, the statistical analysis allows biological treatment effect after adjusting for unwanted influences to be estimated, such as microfluidic device to microfluidic device, row-to-row and holder-to-holder variability. The automatic identification of imaging variability is advantageous, as it allows scientists to better understand where the variability in their experiments is coming from, and from there the scientists adjust experiments to lessen variability. The automatic identification of variability was tested experimentally by scientists imaging microfluidic devices seeded with liver cells. Surprisingly, it was found by the scientists that the highest variability came from microfluidic device to microfluidic device, as opposed to holder-to-holder and row-to-row variability. With that information at hand, the scientists can better target that area of variability and revisit microfluidic device fabrication and seeding in order to decrease variability.

The development of an intelligent, high-content scanning workflow to microfluidic devices seeded with liver cells has been conducted as an exemplary embodiment of the invention. A microfluidic device seeded with exemplary liver cell types is used, in one embodiment, to recapitulate the physiological environment of the liver. Any organ name followed by chip, such as Liver Chip, Kidney Chip, Gut Chip, Blood-Brain-Barrier Chip, etc., signifies a microfluidic device seeded with that variety of exemplary cell types in order to recapitulate the physiological environment of that particular organ. Microfluidic devices seeded with liver cells have enormous potential as predictive models for Drug-Induced Liver Injury (DILI) which remains a major cause of drug attrition during drug discovery and development. A framework has been developed for the statistical analysis of microfluidic devices seeded with liver cells that not only reduces bias and variability but also ensures that the questions posed by the study can be robustly answered. In some embodiments, the sample size is large enough to detect a hepatotoxic effect with an appropriate experimental power. Accurate sample size calculations minimize experimental cost since they prevent scientists from running studies that are inconclusive or use too many microfluidic devices. The mass content microscopy analysis further identifies different sources of variability and allows scientists to test for adverse effects while controlling for unwanted influences. The high-content microscopy and related statistics framework presented herein has been used to design and prosecute a study to evaluate the hepatotoxic effects of an active compound that is in clinical development.

To demonstrate broader embodiments of this workflow for different microfluidic device formats, a preliminary investigation of a microfluidic device to be seeded with kidney cells from a different manufacturer, designed to generate a complete tubular structure with unidirectional perfusion, has been conducted. The microfluidic device seeded with kidney cells differs from the microfluidic device seeded with liver cells firstly in thickness, and also as it incorporates a glass window on the bottom face designed to facilitate imaging. In one embodiment, the microfluidic device seeded with kidney cells has an overall size still compatible with the bespoke microfluidic device adaptors for use with a microscope, such as a confocal microscope.

Exemplary embodiments are presented below in order to elucidate model uses of the inventions presented herein.

One exemplary embodiment of the present invention is a method of imaging microfluidic devices comprising: (a) providing a microfluidic device comprising a membrane, said membrane separating two microfluidic channels; (b) providing a microscope capable of image acquisition; (c) taking a first set of microscopic acquisitions; (d) analyzing the first set of microscope acquisitions, determining a focal height and locating a standard coordinate system, wherein the coordinate system is located based on the location of the membrane within the microfluidic device; (e) and taking a second set of microscopic acquisitions based on the coordinate system located in the first set of microscopic acquisitions.

In an exemplary embodiment, the microscope may be a confocal microscope. In one embodiment, the first set of microscopic acquisitions are low-resolution. In one embodiment, the second set of microscopic acquisitions are high-resolution. In one exemplary embodiment, the microfluidic device is seeded with cells. In one embodiment, the second set of microscope acquisitions are used to evaluate the effect of an agent on the cells. In one embodiment, the agent is a pharmaceutical. In one embodiment, the cells are cultured for more than seven days. In one embodiment, the cells are located in the channels separated by the membrane. In one embodiment, the second set of microscopic acquisitions comprises a three-dimensional acquisition. In one embodiment, the three-dimensional acquisition comprises an endothelial cell layer and hepatocyte cell layer together, separated by the membrane. In one embodiment, the cells are liver cells. In one embodiment, the liver cells are hepatocytes and sinusoidal endothelial cells. In one embodiment, the hepatocytes and sinusoidal endothelial cells are human hepatocytes and human sinusoidal endothelial cells. In one embodiment, the cells are kidney cells. In one embodiment, the microscopic acquisitions are of individual cells. In one embodiment, the channels of the microfluidic device are coated with a mixture of extracellular matrix. In one embodiment, the method further comprises applying flow to the channels. In one embodiment, the flow exerts shear stress on the cells. In one embodiment, the method further comprises identifying the presence of cells in the microfluidic device. In one embodiment, the method further comprises identifying the presence of nuclear stains on the cells in the microfluidic device. In one embodiment, the method further comprises identifying membrane markers between the cells in the microfluidic device. In one embodiment, the membrane markers are tight junction markers. In one embodiment, the tight junction markers are zonula occludens-1 (ZO-1) markers. In one embodiment, the tight junction markers are cadherin markers. In one embodiment, the cadherin markers are epithelial cadherin markers. In one embodiment, the method further comprises identifying the presence of a gradient along the length microfluidic device. In one embodiment, the gradients are identified downstream in the microfluidic device channels. In one embodiment, the gradients are identified upstream in the microfluidic device channels. In one embodiment, the gradient is a change in the number of metabolites. In one embodiment, the gradient is an oxygen gradient. In one embodiment, the gradient is a change in the number of nuclei present. In one embodiment, the method further comprises identifying the presence of α-SMA. In one embodiment, the method further comprises identifying lipid accumulation. In one embodiment, the method further comprises identifying bile canaliculi. In one embodiment, the cells are identified using geometric criteria. In one embodiment, the geometric criteria are selected from a list comprising of size, circularity, eccentricity and solidity.

One exemplary embodiment of the present invention is a method of imaging microfluidic devices comprising: (a) providing a microfluidic device comprising a membrane, said membrane separating two microfluidic channels; (b) providing a microscope capable of image acquisition; (c) taking a first set of microscopic acquisitions; (d) analyzing the first set of microscope acquisitions, determining a focal height and locating a standard coordinate system, wherein the coordinate system is located based on the location of the membrane within the microfluidic device; (e) and taking a second set of microscopic acquisitions based on the coordinate system located in the first set of microscopic acquisitions.

In one embodiment, the coordinate system is located based on pores in the membrane. In one embodiment, the coordinate system is located based on the location of a first surface of the membrane. In one embodiment, the coordinate system is located based on the location of a second surface of the membrane. In one embodiment, the coordinate system is located based on a first and second surface of the membrane. In one embodiment, the method further comprises identifying the presence of cells in the microfluidic device. In one embodiment, the method further comprises identifying the presence of nuclear stains on the cells in the microfluidic device. In one embodiment, the method further comprises identifying membrane markers between the cells in the microfluidic device. In one embodiment, wherein the membrane markers are tight junction markers. In one embodiment, the tight junction markers are zonula occludens-1 (ZO-1) markers. In one embodiment, wherein the tight junction markers are cadherin markers. In one embodiment, wherein the cadherin markers are epithelial cadherin markers. In one embodiment, the method further comprises identifying the presence of a gradient along the length microfluidic device. In one embodiment, wherein the gradients are identified downstream in the microfluidic device channels. In one embodiment, wherein the gradients are identified upstream in the microfluidic device channels. In one embodiment, wherein the gradient is a change in the number of metabolites. In one embodiment, wherein the gradient is an oxygen gradient. In one embodiment, wherein the gradient is a change in the number of nuclei present. In one embodiment, the method further comprises identifying the presence of α-SMA. In one embodiment, the method further comprises identifying lipid accumulation. In one embodiment, the method further comprises identifying biocanaliculi. In one embodiment, the cells are identified using geometric criteria. In one embodiment, the geometric criteria are selected from a list comprising of size, circularity, eccentricity and solidity. In one embodiment, the microscope is a confocal microscope. In one embodiment, the first set of microscopic acquisitions are low-resolution. In one embodiment, the second set of microscopic acquisitions are high-resolution. In one embodiment, the microfluidic device is seeded with cells. In one embodiment, the second set of microscope acquisitions are used to evaluate the effect of an agent on the cells. In one embodiment, the agent is a pharmaceutical. In one embodiment, the cells are cultured for more than seven days. In one embodiment, the cells are located in the channels separated by the membrane. In one embodiment, the second set of microscopic acquisitions comprises a three-dimensional acquisition. In one embodiment, the three-dimensional acquisition comprises an endothelial cell layer and hepatocyte cell layer together, separated by the membrane. In one embodiment, the cells are liver cells. In one embodiment, the liver cells are hepatocytes and sinusoidal endothelial cells. In one embodiment, the hepatocytes and sinusoidal endothelial cells are human hepatocytes and human sinusoidal endothelial cells. In one embodiment, the cells are kidney cells. In one embodiment, the microscopic acquisitions are of individual cells. In one embodiment, the channels of the microfluidic device are coated with a mixture of extracellular matrix. In one embodiment, the method further comprises applying flow to the channels. In one embodiment, the flow exerts shear stress on the cells.

An exemplary method of analyzing cellular phenotype changes following agent exposure comprises: (a) providing one or more microfluidic device comprising microchannels, said microchannels comprising microchannel walls; (b) providing a microscope capable of image acquisition; (c) treating a number of the microfluidic devices an agent and a number of microfluidic devices with a control media; (d) taking a first set of microscopic acquisitions; (e) analyzing the first set of microscope acquisitions and locating a standard coordinate system, wherein the coordinate system is located based on the location of the microchannel walls within the microfluidic device; (f) taking a second set of microscopic acquisitions based on the coordinate system located in the first set of microscopic acquisitions; (g) making endpoint measurements of the acquisitions; (h) fitting a Bayesian linear regression model to the measurements; (i) estimating a linear field effect based on the Bayesian linear regression; and (j) comparing the linear field effect from microfluidic devices treated with an agent verses microfluidic device treated with a control media.

In one embodiment, the microscope is a confocal microscope. In one embodiment, the first set of microscopic acquisitions are low-resolution. In one embodiment, the second set of microscopic acquisitions are high-resolution. In one embodiment, the microfluidic device is seeded with cells. In one embodiment, the second set of microscope acquisitions are used to evaluate the effect of an agent on the cells. In one embodiment, the agent is a pharmaceutical. In one embodiment, the cells are cultured for more than seven days. In one embodiment, the cells are located in the channels separated by the membrane. In one embodiment, the second set of microscopic acquisitions comprises a three-dimensional acquisition. In one embodiment, the three-dimensional acquisition comprises an endothelial cell layer and hepatocyte cell layer together, separated by the membrane. In one embodiment, the cells are liver cells. In one embodiment, the liver cells are hepatocytes and sinusoidal endothelial cells. In one embodiment, the hepatocytes and sinusoidal endothelial cells are human hepatocytes and human sinusoidal endothelial cells. In one embodiment, the cells are kidney cells. In one embodiment, the microscopic acquisitions are of individual cells. In one embodiment, the channels of the microfluidic device are coated with a mixture of extracellular matrix. In one embodiment, the method further comprises applying flow to the channels. In one embodiment, the flow exerts shear stress on the cells.

An exemplary method of analyzing cellular phenotype changes following agent exposure comprises: (a) providing one or more microfluidic device comprising a membrane, said membrane separating two microfluidic channels, each channel seeded with cells; (b) providing a microscope capable of image acquisition; (c) treating a number of the microfluidic devices an agent and a number of microfluidic devices with a control media; (d) taking a first set of microscopic acquisitions; (e) analyzing the first set of microscope acquisitions and locating a standard coordinate system, wherein the coordinate system is located based on the location of the membrane within the microfluidic device; (f) taking a second set of microscopic acquisitions based on the coordinate system located in the first set of microscopic acquisitions; (g) making endpoint measurements of the acquisitions; (h) fitting a Bayesian linear regression model to the measurements; (i) estimating a linear field effect based on the Bayesian linear regression; and (j) comparing the linear field effect from microfluidic devices treated with an agent verses microfluidic device treated with a control media.

In one embodiment, the microscope is a confocal microscope. In one embodiment, the first set of microscopic acquisitions are low-resolution. In one embodiment, the second set of microscopic acquisitions are high-resolution. In one embodiment, the microfluidic device is seeded with cells. In one embodiment, the second set of microscope acquisitions are used to evaluate the effect of an agent on the cells. In one embodiment, the agent is a pharmaceutical. In one embodiment, the cells are cultured for more than seven days. In one embodiment, the cells are located in the channels separated by the membrane. In one embodiment, the second set of microscopic acquisitions comprises a three-dimensional acquisition. In one embodiment, the three-dimensional acquisition comprises an endothelial cell layer and hepatocyte cell layer together, separated by the membrane. In one embodiment, the cells are liver cells. In one embodiment, the liver cells are hepatocytes and sinusoidal endothelial cells. In one embodiment, the hepatocytes and sinusoidal endothelial cells are human hepatocytes and human sinusoidal endothelial cells. In one embodiment, the cells are kidney cells. In one embodiment, the microscopic acquisitions are of individual cells. In one embodiment, the channels of the microfluidic device are coated with a mixture of extracellular matrix. In one embodiment, the method further comprises applying flow to the channels. In one embodiment, the flow exerts shear stress on the cells.

One exemplary embodiment of the present invention is a method of flowing a plurality of lipid nanoparticles (LNP) for delivering nucleic acid sequences to cells in a microfluidic device, comprising, a) providing, i) a plurality of lipid nanoparticles (LNP) comprising nucleic acid sequences; ii) a microfluidic device comprising a membrane, said membrane separating two microfluidic channels, wherein at least one channel is seeded with cells, and b) flowing said LNPs into one of said channels for delivering said nucleic acid sequences to said cells. In one embodiment, said LNPs comprise Adeno-Associated Virus (AAV) sequences. In one embodiment, said Adeno-Associated Virus (AAV) sequences are selected from the group of serotypes consisting of AAV2, AAV8, and AAV9. In one embodiment, said LNPs are encapsulated. In one embodiment, said LNPs are decorated. In one embodiment, said delivery results in transfecting said cells with said nucleic acid sequences. In one embodiment, said nucleic acid sequences are selected from the group consisting of ribonucleic acid (RNA), messenger ribonucleic acid (mRNA), and deoxyribonucleic acid (DNA). In one embodiment, said nucleic acid sequences are silencing ribonucleic acids (RNA) selected from the group consisting of small interfering RNA (siRNA) and RNA interference (RNAi). In one embodiment, said nucleic acid sequences encode a green fluorescent protein (GFP) transgene. In one embodiment, said contacting is in the same channel as said cells. In one embodiment, said cells are attached to said membrane. In one embodiment, said cells are hepatocytes. In one embodiment, said hepatocytes are selected from the group consisting of human. monkey, rat and mouse. In one embodiment, said hepatocytes cultured for more than three days. In one embodiment, said hepatocytes cultured for more than seven days. In one embodiment, said hepatocytes are cultured for more than nine days. In one embodiment, said hepatocytes are expressing asialoglycoprotein receptor 1 (ASGR1). In one embodiment, said hepatocytes are expressing asialoglycoprotein receptor 1 (ASGR1) after nine days of culture. In one embodiment, said hepatocytes are expressing asialoglycoprotein receptor 1 (ASGR1) on day 10 of culture. In one embodiment, said nucleic acid sequences encode silencing molecules for reducing expression of asialoglycoprotein receptor 1 (ASGR1). In one embodiment, said microfluidic device is seeded with a second cell type. In one embodiment, said second cell type is a population of endothelial cells. In one embodiment, said method further provides a microscope capable of image acquisition; and taking a first set of microscopic acquisitions prior to said contacting, analyzing the first set of microscope acquisitions, determining a focal height and locating a standard coordinate system, wherein the coordinate system is located based on the location of the membrane within the microfluidic device. In one embodiment, said method further comprises identifying the presence of said cells in the microfluidic device. In one embodiment, said method further comprises identifying the presence of nuclear stains on said cells in the microfluidic device. In one embodiment, said method further comprises taking a second set of microscopic acquisitions after said contacting based on the coordinate system located in the first set of microscopic acquisitions. In one embodiment, said second set of microscope acquisitions are used to evaluate the effect of said delivery of nucleic acids on said cells. In one embodiment, said method further comprises identifying a membrane marker between the cells in the microfluidic device. In one embodiment, said membrane marker is asialoglycoprotein receptor 1 (ASGR1). In one embodiment, said nucleic acid sequences are a pharmaceutical.

One exemplary embodiment of the present invention is a method of flowing a plurality of lipid nanoparticles (LNP) for delivering nucleic acid sequences to cells in a microfluidic device for analyzing cellular phenotype changes following nucleic acid delivery to a cell comprising, a) providing, i) a plurality of lipid nanoparticles (LNP) comprising nucleic acids; ii) a plurality of lipid nanoparticles (LNP) without nucleic acids; iii) one or more microfluidic device comprising a membrane, said membrane separating two microfluidic channels, wherein at least one channel is seeded with cells in said devices, and iv) a microscope capable of image acquisition; and b) flowing said LNPs comprising nucleic acids into said channel of at least one said microfluidic device for delivering said nucleic acid sequences to said cells and flowing said LNPs without nucleic acids into at least a second microfluidic device; c) taking a first set of microscopic acquisitions; d) analyzing the first set of microscope acquisitions and locating a standard coordinate system, wherein the coordinate system is located based on the location of the membrane within the microfluidic device; e) taking a second set of microscopic acquisitions based on the coordinate system located in the first set of microscopic acquisitions; f) making endpoint measurements of the acquisitions; g) fitting a Bayesian linear regression model to the measurements; h) estimating a linear field effect based on the Bayesian linear regression; and i) comparing the linear field effect from microfluidic devices treated with said LNP comprising said nucleic acid verses said microfluidic device treated with said control LNP without said nucleic acid.

In a further embodiment, the present invention contemplates a method of imaging microfluidic devices comprising: (a) providing a microfluidic device comprising a membrane, said membrane separating two microfluidic channels; (b) providing a microscope capable of image acquisition; (c) taking a first set of microscopic image acquisitions; (d) determining a focal height and locating a standard coordinate system from said first set of microscopic image acquisitions, wherein the coordinate system is located based on the location of the membrane within the microfluidic device; and (e) taking a second set of microscopic image acquisitions based on the coordinate system located in the first set of microscopic acquisitions. It is not intended that the present invention be limited to a particular type of microscope; however, one preferred microscope is a confocal microscope. In one embodiment, the first set of microscopic acquisitions are low-resolution. In one embodiment, the second set of microscopic acquisitions are high-resolution. In a preferred embodiment, the microfluidic device is seeded with cells. In one embodiment, the second set of microscope acquisitions are used to evaluate the effect of an agent on the cells. In one embodiment, the agent is a pharmaceutical or test candidate. In one embodiment, the cells are cultured for more than seven days. In one embodiment, the cells are located in the channels separated by the membrane. In one embodiment, the second set of microscopic acquisitions comprises a three-dimensional acquisition. In one embodiment, the three-dimensional acquisition comprises an endothelial cell layer and hepatocyte cell layer together, separated by the membrane. In one embodiment, the cells are lung cells. In one embodiment, the cells are ciliated cells. In one embodiment, the cells are liver cells. In one embodiment, the liver cells are hepatocytes and sinusoidal endothelial cells. In one embodiment, the hepatocytes and sinusoidal endothelial cells are human hepatocytes and human sinusoidal endothelial cells. In one embodiment, the cells are kidney cells. In one embodiment, the microscopic acquisitions are of individual cells. In one embodiment, the channels of the microfluidic device are coated with a mixture of extracellular matrix. In one embodiment, the method further comprises applying flow to the channels. In one embodiment, the second set of acquisitions, guided by the coordinate system, comprises Z stack slices through different layers of the microfluidic device. In one embodiment, the method further comprises identifying the presence of cells in the microfluidic device. In one embodiment, the method further comprises identifying the presence of nuclear stains on the cells in the microfluidic device. In one embodiment, the method further comprises identifying membrane markers between the cells in the microfluidic device. In one embodiment, the membrane markers are tight junction markers. In one embodiment, the tight junction markers are zonula occludens-1 (ZO-1) markers. In one embodiment, the tight junction markers are cadherin markers. In one embodiment, the cadherin markers are epithelial cadherin markers. In one embodiment, the method further comprises identifying the presence of a gradient along the length microfluidic device. In one embodiment, the gradients are identified downstream in the microfluidic device channels. In one embodiment, the gradients are identified upstream in the microfluidic device channels. In one embodiment, the gradient is a change in the number of metabolites. In one embodiment, the gradient is an oxygen gradient. In one embodiment, the gradient is a change in the number of nuclei present. In one embodiment, the method further comprises identifying the presence of α-SMA. In one embodiment, the method further comprises identifying lipid accumulation. In one embodiment, the method further comprises identifying bile canaliculi. In one embodiment, the cells are identified using geometric criteria. In one embodiment, the geometric criteria are selected from a list comprising of size, circularity, eccentricity and solidity.

In a further embodiment, the present invention contemplates a method of imaging microfluidic devices comprising: (a) providing a microfluidic device comprising a membrane, said membrane separating two microfluidic channels; (b) providing a microscope capable of image acquisition; (c) taking a set of low resolution microscopic image acquisitions; (d) locating a standard coordinate system using said set of low resolution image acquisitions, wherein the coordinate system is located based on the location of the membrane within the microfluidic device; and (e) taking a set of high resolution microscopic acquisitions based on the coordinate system located in the first set of microscopic acquisitions. In one embodiment, the coordinate system is located based on pores in the membrane. In one embodiment, the coordinate system is located based on the location of a first surface of the membrane. In one embodiment, the coordinate system is located based on the location of a second surface of the membrane. In one embodiment, the coordinate system is located based on a first and second surface of the membrane. In one embodiment, the method further comprises identifying the presence of cells in the microfluidic device. In one embodiment, the method further comprises identifying the presence of nuclear stains on the cells in the microfluidic device. In one embodiment, the method further comprises identifying membrane markers between the cells in the microfluidic device. In one embodiment, the membrane markers are tight junction markers. In one embodiment, the tight junction markers are zonula occludens-1 (ZO-1) markers. In one embodiment, the tight junction markers are cadherin markers. In one embodiment, the cadherin markers are epithelial cadherin markers. In one embodiment, the method further comprises identifying the presence of a gradient along the length microfluidic device. In one embodiment, the gradients are identified downstream in the microfluidic device channels. In one embodiment, the gradients are identified upstream in the microfluidic device channels. In one embodiment, the gradient is a change in the number of metabolites. In one embodiment, the gradient is an oxygen gradient. In one embodiment, the gradient is a change in the number of nuclei present. In one embodiment, the method further comprises identifying the presence of α-SMA. In one embodiment, the method further comprises identifying lipid accumulation. In one embodiment, the method further comprises identifying bile canaliculi. In one embodiment, the cells are identified using geometric criteria. In one embodiment, the geometric criteria are selected from a list comprising of size, circularity, eccentricity and solidity. In one embodiment, the microscope is a confocal microscope. In one embodiment, the microfluidic device is seeded with cells. In one embodiment, the high resolution set of microscopic image acquisitions is used to evaluate the effect of an agent on the cells. In one embodiment, the agent is a pharmaceutical. In one embodiment, the cells are cultured for more than seven days. In one embodiment, the cells are located in the channels separated by the membrane. In one embodiment, the high resolution set of microscopic acquisitions comprises a three-dimensional acquisition. In one embodiment, the three-dimensional acquisition comprises an endothelial cell layer and hepatocyte cell layer together, separated by the membrane. In one embodiment, the cells are lung cells. In one embodiment, the cells are ciliated. In one embodiment, the cells are liver cells. In one embodiment, the liver cells are hepatocytes and sinusoidal endothelial cells. In one embodiment, the hepatocytes and sinusoidal endothelial cells are human hepatocytes and human sinusoidal endothelial cells. In one embodiment, the cells are kidney cells. In one embodiment, the microscopic acquisitions are of individual cells. In one embodiment, the channels of the microfluidic device are coated with a mixture of extracellular matrix. In one embodiment. the method further comprises applying flow to the channels (e.g. wherein the flow exerts shear stress on the cells).

In still another embodiment, the present invention contemplates a method of analyzing cellular phenotype changes following agent exposure comprising: (a) providing a plurality of microfluidic devices comprising cells in microchannels, said microchannels comprising microchannel walls; (b) providing a microscope capable of image acquisition; (c) treating a number of said microfluidic devices with an agent and a number of said microfluidic devices with a control media; (d) taking a first set of microscopic acquisitions; e) locating a standard coordinate system using the first set of microscope acquisitions, wherein the coordinate system is located based on the location of the microchannel walls within the microfluidic device; (f) taking a second set of microscopic acquisitions based on the coordinate system located in the first set of microscopic acquisitions; (g) making endpoint measurements of the acquisitions; (h) fitting a regression model to the measurements; (i) estimating a field effect based on the regression; and (j) comparing the field effect from microfluidic devices treated with an agent verses microfluidic device treated with a control media. In one embodiment, said regression model is a Bayesian linear regression model. In one embodiment, said field effect is a linear field effect. In one embodiment, the microscope is a confocal microscope. In one embodiment, the first set of microscopic acquisitions are low-resolution. In one embodiment, the second set of microscopic acquisitions are high-resolution. In one embodiment, the agent is a pharmaceutical. In one embodiment, the cells are cultured for more than seven days. In one embodiment, the second set of microscopic acquisitions comprises a three-dimensional acquisition. In one embodiment, the three-dimensional acquisition comprises an endothelial cell layer and hepatocyte cell layer together, separated by the membrane. In one embodiment, the cells are lung cells. In one embodiment, the cells are ciliated cells. In one embodiment, the cells are liver cells. In one embodiment, the liver cells are hepatocytes and sinusoidal endothelial cells. In one embodiment, the hepatocytes and sinusoidal endothelial cells are human hepatocytes and human sinusoidal endothelial cells. In one embodiment, the cells are kidney cells. In one embodiment, the microscopic acquisitions are of individual cells. In one embodiment, the method further comprises applying flow to the channels. In still another embodiment, the present invention contemplates a method of imaging, comprising: a) providing a microfluidic device comprising cells stained for α-SMA and a microscope capable of image acquisition; b) taking a first round of image acquisitions of said cells; c) calculating coordinates based on the first round of image acquisitions; and d) taking a second round of image acquisitions of said cells based on the coordinates of step c). In one embodiment, said cells stained for α-SMA have a non-zero baseline when fluorescently imaged. In one embodiment, said second round of image acquisitions distinguishes between background and α-SMA related fluorescence. In yet another embodiment, the present invention contemplates a method of imaging, comprising: (a) providing a microfluidic device comprising cells stained for lipid accumulation and a microscope capable of image acquisition; (b) taking a first round of image acquisitions of said cells; (c) calculating coordinates based on the first round of image acquisitions; and (d) taking a second round of image acquisitions of said cells based on the coordinates of step c). In one embodiment, said cells stained for α-SMA have a non-zero baseline when fluorescently imaged. In one embodiment, said second round of image acquisitions distinguishes between background and lipid accumulation related fluorescence. In still another embodiment, the present invention contemplates a method of imaging, comprising: (a) providing a microfluidic device comprising liver cells stained for bile canaliculi and a microscope capable of image acquisition; (b) taking a first round of image acquisitions of said cells; (c) calculating coordinates based on the first round of image acquisitions; and (d) taking a second round of image acquisitions of said cells based on the coordinates of step c). In one embodiment, said second round of image acquisitions distinguishes between background and bile canaliculi related fluorescence. In one embodiment, the second set of microscopic acquisitions comprises a three-dimensional acquisition. In one embodiment, the three-dimensional acquisition comprises an endothelial cell layer and hepatocyte cell layer together, separated by the membrane. In one embodiment, the method further comprises applying flow to the channels.

In yet another embodiment, the present invention contemplates a statistical method of analyzing microfluidic device acquisitions in order to decouple sources of variability comprising: (a) randomizing the order in which microfluidic devices are imaged; b) taking images according to the randomizing of step a); (c) fitting a regression model to the images; and (d) estimating a parameter, said parameter selected from the group consisting of treatment effects, time effects, and microfluidic device variability. In one embodiment, said regression model is a Bayesian linear regression model.

### Definitions

The term “microfluidic” as used herein, relates to components where moving fluid is constrained in or directed through one or more channels wherein one or more dimensions are 1 mm or smaller (microscale). Microfluidic devices are described in the U.S. Pat. No. 8,647,861, and the International Patent App. No. PCT/US2014/071611, the contents of each are incorporated herein by reference in it's entirety, (such microfluidic devices are also referred to herein as “chips”). Microfluidic channels may be larger than microscale in one or more directions, though the channel(s) will be on the microscale in at least one direction. In some instances, the geometry of a microfluidic channel may be configured to control the fluid flow rate through the channel (e.g. increase channel height to reduce shear). Microfluidic channels can be formed of various geometries to facilitate a wide range of flow rates through the channels.

Examples of microfluidic devices include but are not limited to: WO 2010/009307, Organ Mimic Device With Microchannels And Methods Of Use And Manufacturing Thereof; and examples of microfluidic devices having open tops: WO2017096297; WO2013086486, herein incorporated by reference in their entireties. Examples of microfluidic devices including airway cells for use as described herein, specifically including microfluidic devices for use in high content imaging, e.g. lung, alveolar cells, respiratory cells, small airway cells, bronchial cells, etc. include but are not limited to: WO2013086486; WO2016010861; WO2017096297, herein incorporated by reference in their entireties. In some embodiments, microfluidic devices provided for use in high content imaging comprise airway cells have an air-liquid interface. In some embodiments, microfluidic devices provided for use in high content imaging comprise airway cells do not have an air-liquid interface, e.g., while freshly seeded cells are undergoing initial divisional growth and differentiation.

The phrases “connected to,” “coupled to,” “in contact with,” and “in communication with” as used herein, refer to any form of interaction between two or more entities, including mechanical, electrical, magnetic, electromagnetic, fluidic, and thermal interaction. For example, in one embodiment, channels in a microfluidic device are in fluidic communication with a fluid source such as a fluid reservoir. Two components may be coupled to each other even though they are not in direct contact with each other. For example, two components may be coupled to each other through an intermediate component (e.g. tubing or other conduit). Thus, a working fluid in a rigid container can be in fluidic communication with a working fluid reservoir via tubing or other conduit.

The term “channels” as used herein, are pathways (whether straight, curved, single, multiple, in a network, etc.) through a medium (e.g., silicon) that allow for movement of liquids and gasses. Channels thus can connect other components, i.e., keep components “in communication” and more particularly, “in fluidic communication” and still more particularly, “in liquid communication.” Such components include, but are not limited to, liquid-intake ports and gas vents. Microchannels are channels with dimensions less than 1 millimeter and greater than 1 micron.

“Microchannels” are channels with dimensions less than 1 millimeter and greater than 1 micron. Additionally, the term “microfluidic” as used herein relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances, the geometry of a microfluidic channel may be conFIG.d to control the fluid flow rate through the channel (e.g. increase channel height to reduce shear). Microfluidic channels can be formed of various geometries to facilitate a wide range of flow rates through the channels. One portion of a microchannel can be a membrane. For example, the floor of a microchannel can comprise a membrane, including a porous membrane. The microchannel (or portion thereof) or membrane can be coated with substances such as various cell adhesion promoting substances or ECM proteins, such as fibronectin, laminin or various collagen types or combinations thereof. For example, endothelial cells can attach to a collagen coated microchannel.

The present invention contemplates a variety of “microfluidic devices.” The methods described herein for the use of microfluidic devices and for perfusing microfluidic devices are not limited to the particular embodiments of microfluidic devices described herein, and may be applied generally to microfluidic devices, e.g. devices having one or more microchannels and ports.

“High-content” refers to the ability to image many microfluidic devices in a shortened period of time as compared to manual microscopy of each microfluidic device.

### BRIEF DESCRIPTIONS OF DRAWINGS

Exemplary embodiments are illustrated in referenced FIG.s. It is intended that the embodiments and FIG.s disclosed herein are to be considered illustrative rather than restrictive.

The file of this patent contains at least one drawing executed in color. Copies of this patent with color drawings will be provided by the Patent and Trademark Office upon request and payment of the necessary fee.

FIGS. 1A-1B show macroscopic and microscopic depictions of a microfluidic device seeded with liver cells. FIG. 1A shows a schematic of microfluidic devices seeded with liver cells illustrating dimensions; FIG. 1B shows that each microfluidic device incorporates a Hepatocyte and Liver Sinusoidal Endothelial Cell (LSEC) interface held within Extracellular Matrix (ECM) (purple) coated membrane (grey). Incorporation of microfluidics allows physiological flow of media/drug treatment across the cell layers.

FIG. 2 shows the design and 3D printing of adaptors that allow microfluidic device compatibility with plate based, automated microscopes. Left, shows a schematic of microfluidic device seeded with liver cells. Middle, shows an adaptor design. Right, microfluidic devices seeded with liver cells within adaptors to fit footprint of standard Multi-well plate.

FIGS. 3A-3B show one embodiment of an imaging workflow and intelligent scanning strategy used on a microfluidic device seeded with liver cells. FIG. 3A shows one embodiment of an outline of a first pass imaging workflow, or the first series of acquisitions, and intelligent scanning strategy of microfluidic device seeded with liver cells. In one embodiment, the automated microscope first performs a low resolution (4×) bright field scan of the microfluidic devices within each adaptor. In the embodiment depicted, the adaptor holds 8 microfluidic devices. In the embodiment depicted, the first set of low-resolution acquisitions are followed by the automatic launch of an analysis script that stitches the images together and defines X-Y coordinates for fields of view required for high-resolution imaging. FIG. 3B shows an outline of second pass imaging, or the second set of acquisitions, which in this embodiment are high-resolution. CV7000 microscope acquires a high resolution (20×) Z stack at the XY coordinates defined by first pass imaging. Image processing accurately separates out the cell layers ready for image analysis

FIGS. 4A-4C show exemplary confocal images taken at 20× magnification. Images are representative and taken from the mid region of each microfluidic device. Images shown include markers of F-actin morphology (Phalloidin), proliferation (Ki67), mitochondrial structure (ATPB—ATP synthase beta subunit), apoptosis (CC3—Cleaved Caspase 3) and Nuclei (Hoechst). FIG. 4A shows images captured from a microfluidic device treated with vehicle control (DMSO). FIGS. 4B-4C show images are taken from the mid region of microfluidic devices and show markers of F-actin morphology (Phalloidin), apoptosis (CC3) and Nuclei (Hoechst). Images were captured from microfluidic devices treated with vehicle control (DMSO) or staurosporine (10 μM) for 6 hours. FIG. 4B shows the microfluidic devices treated with vehicle control (DMSO). FIG. 4C shows microfluidic devices treated with staurosporine (10 μM).

FIGS. 5A-5B show Bayesian analysis of a test compound to understand the variability in the data allowing the decoupling of the variability due to technical error (in blue) from experimental conditions (in green). Two examples are given, as shown in FIGS. 5A and 5B. FIG. 5A shows analysis based on LSEC cell count. FIG. 5B shows analysis based on CC3 (Cleaved Caspase 3) signal in hepatocytes.

FIGS. 6A-6E show recapitulation of species-specific drug toxicities in rat, dog, and human microfluidic liver devices. FIG. 6A is a schematic of a microfluidic device seeded with liver cells that recapitulates complex liver microarchitecture. Primary hepatocytes in the upper parenchymal channel in ECM sandwich format and NPCs (e.g. LSECs, Kupffer, and stellate cells) on the opposite side of the same membrane in the lower vascular channel. FIG. 6B shows albumin secretions after daily administration of bosentan at 1, 3, 10, 30, and 100 μM for 3 days in dual-cell (hepatocyte and LSECs) microfluidic devices seeded with human liver cells and plates (hepatocyte sandwich monoculture) and for 7 days in dual-cell dog and rat microfluidic device liver systems and plates (n=3 independent microfluidic devices and plate wells). FIG. 6C shows quantification of % CLF-positive area in bile canaliculi (BC) from the parenchymal channel after bosentan treatment at 30 μM for 7 days in microfluidic devices seeded with human liver cells. Mann-Whitney U test (n=3 independent microfluidic devices with 3 randomly selected different areas per microfluidic device, see detailed description on the analysis herein). FIG. 6D shows representative images of CLF (green, BSEP substrate) and BSEP (red, DAPI in blue) from the parenchymal channel. FIG. 6E shows quantification of BSEP-positive area and fold change of BSEP gene expression. Mann-Whitney U test (n=3 independent microfluidic devices). Scale bar, 20 μm. *P<0.05, **P<0.01, ****P<0.0001. Error bars present mean±SEM.

FIGS. 7A-7C shows detection of hepatocellular injury and release of various DILI biomarkers using quadruple-cell human microfluidic device liver systems. FIG. 7A shows total GSH and ATP levels from the parenchymal and vascular channels after daily administration of APAP at 0.5, 3, and 10 mM for 7 days in microfluidic devices seeded with human liver cells. FIG. 7B shows representative images of ROS levels (magenta, CellROX) after daily administration of APAP at 0.5, 3, and 10 mM and co-administration of 3 mM of APAP and 200 μM of BSO for 7 days in microfluidic devices seeded with human liver cells and quantification of number of CellROX-positive events per field of view. Kruskal-Wallis tests (n=3 independent microfluidic devices with 3 to 5 randomly selected different areas per microfluidic device). Scale bar, 100 gm. FIG. 7C shows albumin, aGST, and miR-122 secretions from the parenchymal channel after APAP treatment for 7 days in microfluidic devices seeded with human liver cells. Dunnett's multiple comparisons test (n=10˜18 independent microfluidic devices for albumin, n=3˜9 independent microfluidic devices for the rest). *P<0.05, **P<0.01, ***P<0.001 , ****P<0.0001. Error bars present mean±SEM.

FIGS. 8A-8C shows detection of Kupffer cell depletion, steatosis and fibrosis in microfluidic devices seeded with human liver cells. FIG. 8A shows representative images of lipid droplets (yellow, Nile red and DAPI in blue) from the parenchymal channel and alpha-SMA (green) from the vascular channel to indicate activated stellate cells after daily administration of MTX at 1, 10, and 30 μM for 7 days in microfluidic devices seeded with human liver cells. FIG. 8B shows quantification of Nile red-positive events per field of view and α-SMA-positive cells per field of view. Kruskal-Wallis tests (n=3 independent microfluidic devices with 3-5 randomly selected different areas per microfluidic device). FIG. 8C shows albumin secretion from the parenchymal channel and IP-10 secretion from the vascular channel after MTX treatment for 7 days and 1 day respectively in microfluidic devices seeded with human liver cells. Scale bar, 100 μm. *P<0.05, **P<0.01, ***P<0.001 , ****P<0.0001. Error bars present mean±SEM.

FIGS. 9A-9C shows a comparison of species differences in steatosis using rat and human microfluidic device liver systems following fialuridine (FIAU) treatment. FIG. 9A shows representative images of lipid droplets (yellow, Nile red and DAPI in blue) from the parenchymal channel after daily administration of FIAU at 1, 10, and 30 μM for 10 days in rat and human microfluidic device liver systems and quantification of Nile red intensity. FIG. 9B shows albumin secretions as % control after FIAU treatment for 7 days in rat and human microfluidic device liver systems. FIG. 9C shows Mir-122, alpha-GST, and keratin 18 secretions after FIAU treatment for 10 days in microfluidic devices seeded with human liver cells. Dunnett's multiple comparisons test (n=3 independent microfluidic devices). Scale bar, 100 μm. *P<0.05, **P<0.01, ****P<0.0001. Error bars present mean±SEM.

FIGS. 10A-10C depicts how to identify risk for idiosyncratic DILI using microfluidic devices seeded with human liver cells. FIG. 10A shows representative images of CDFDA (green, DAPI in blue) to identify MRP2 transporter activity, TMRM and CellROX (red and cyan respectively, DAPI in blue) to detect mitochondrial depolarization and ROS respectively, and AdipoRed (red, DAPI in blue) to detect lipid droplets after daily administration of TAK-875 at 10 and 30 μM for 8 days or 15 days in microfluidic devices seeded with human liver cells. FIG. 10B shows quantifications of number of CDFDA positive fractions in bile canaliculi area, number of redistributed TMRM fractions and CellROX positive events per field of view after daily administration of TAK-875 at 3, 10, and 30 μM for 15 days in microfluidic devices seeded with human liver cells. Kruskal-Wallis tests (n=3 independent microfluidic devices with 5 randomly selected different areas per microfluidic device). FIG. 10C shows MCP-1 and IL-6 releases from the vascular channel and albumin and keratin 18 secretions from the parenchymal channel after 14 days of TAK-875 treatment in microfluidic devices seeded with human liver cells. Dunnett's multiple comparisons test (n=3 independent microfluidic devices). ****P<0.0001. Error bars present mean±SEM.

FIG. 11 shows exemplary stellate cell activation following TAK-875 treatment. Representative images of aSMA (red, DAPI in blue) to detect activated stellate cells after daily administration of

TAK-875 at 10 or 30 uM for 15 days in human Liver-Chips. Quantifications of % aSMA positive area from the vascular channel. Not significant (n=2 independent chips with 3-5 randomly selected different areas per chip).

FIGS. 12A-12C show morphology and functionality of species-specific dual-cell microfluidic device liver systems. FIG. 12A shows a schematic of the dual-cell microfluidic device liver system that recapitulates complex liver microarchitecture. Primary hepatocytes in the upper channel in ECM sandwich format and LSECs on the opposite side of the same membrane in the lower vascular channel. FIG. 12B shows representative images of hepatocytes (bright-field), CDFDA (green) to visualize bile canaliculi in hepatocytes, MRP2 (green and DAPI in blue) in hepatocytes, and stabilin-1 (red and DAPI in blue) in LSECs after 14 days of culture in human, dog, and rat microfluidic device liver systems and sandwich monoculture plates. Scale bar, 100 μm. FIG. 12C shows albumin and urea secretions in human, dog, and rat microfluidic device liver systems over 2 weeks compared to static sandwich monoculture plates. Dunnett's multiple comparisons test (n=7-20 independent microfluidic devices, n=3-9 independent wells in plate). **P<0.01, ***P<0.001 , ****P<0.0001. Error bars present mean±SEM.

FIG. 13 shows cytochrome P450 enzyme activity in species-specific dual-cell microfluidic device liver systems. Cytochrome P450 enzyme activity in human, dog, and rat microfluidic device liver systems compared to conventional sandwich monoculture plates and fresh hepatocyte suspension over 2 weeks using a cocktail (for dog and rat) or single (for human) probe substrate. Unit: pmol/min/10⁶ cells. Dunnett's or Sidak's multiple comparisons test (n=3 to 20 independent microfluidic devices). *P<0.05, **P<0.01, ***P<0.001 , ****P<0.0001. Error bars present mean±SEM.

FIGS. 14A-14B show a comparison of hepatic functionalities between dual- and quadruple-cell microfluidic device liver systems. FIG. 14A shows a comparison of albumin secretions between dual- and quadruple-cell microfluidic device liver systems from three species models. FIG. 14B shows a comparison of CYP450 enzyme activities between dual- and quadruple-cell microfluidic devices seeded with either rat or human liver cells. Dunnett's or Sidak's multiple comparisons test (n=3 to 4 independent microfluidic devices). ***P<0.001. Error bars present mean±SEM.

FIGS. 15A-15B show detection of glucuronide metabolites of APAP and hepatocellular injury using quadruple-cell human microfluidic device liver systems. FIG. 15A shows APAP glucuronide metabolites formation from upper parenchymal (P) and lower vascular (V) channels after APAP treatment at 3 mM for 20 days from microfluidic devices seeded with human liver cells. (n=4 independent microfluidic devices). FIG. 15B shows representative bright-field images of hepatocytes after daily administration of APAP at 0.5, 3, and 10 mM and co-administration of APAP 3 mM and 200 μM of buthionine sulfoximine (BSO) for 7 days in microfluidic devices seeded with human liver cells.

FIG. 16 is an exemplary microscopic image of the co-culture region of a microfluidic device. The membrane may be identified by the presence of membrane pores.

FIG. 17 is an exemplary microscopic image of the region where one channel may begin or end to overlap a second channel in a microfluidic device. The membrane may be identified by the presence of membrane pores.

FIG. 18 is a representative image of human stellate cells stained by vimentin (green) in the bottom channel of a microfluidic device, specifically an Emulate human Liver Chip.

FIG. 19 is a representative image of a STAB1 (red) and nuclei staining (blue) indicating the presence of LSEC cells in the bottom channel of a microfluidic device, specifically an Emulate human Liver Chip.

FIG. 20 shows exemplary ASGR1 found to be present and localized within hepatocytes after 10 days in culture in the Liver-Chip. Human Liver-Chip—Day 10 in Culture. Pink—ASGR1 protein. Nuclei colored blue (DAPI).

FIG. 21 shows exemplary results after LNPS comprising GFP mRNA were delivered to human hepatocytes in the apical channel. Fluorescent images are shown over time from the apical channel where two types of LNPs comprising GFP mRNA were delivered in the apical channel resulting in LNP-specific GFP expression patterns. No signal was observed in vehicle treated cells or in the cells located in the basal channel under apical administration. Green shows exemplary GFP expression. The lower chart shows exemplary GFP signal over time for LNP#1 and LNP#2.

FIG. 22A shows immunofluorescent images of GFP expression in hepatocytes as dose dependent in Human Liver-Chips, tested as controls (no LNPs), 3×10⁸, 3×10⁹, and 3×10¹⁰ as AAV concentration of genome copies per mL-GC/mL.

FIG. 22B shows immunofluorescent images of GFP expression in hepatocytes as dose dependent in Cynomolgus (monkey) Liver-Chips. AAV concentration (GC/mL).

FIG. 23 demonstrates that 3 AAVs vectors, AA2, AAV8, AAV9, displayed time dependent transduction in cynomolgus and human Liver-Chips, Day 3 vs. Day 5, for 3×10⁸, 3×10⁹, and 3×10¹⁰ AAV genome copies per mL-GC/mL.

FIG. 24 shows an exemplary human Airway chip. Schematic diagram of one embodiment of a human Airway chip with a 3 um pore (e.g., PET) membrane in between airway epithelium and microvascular endothelium (left). Differentiated airway epithelium exhibits continuous tight junctional connections on-chip (e.g., Zo-1+ network of cells). Well-differentiated human airway epithelium generated on-chip contains goblet cells (MUCSAC+ cells) and demonstrates extensive coverage of ciliated cells labeled for alpha-tubulin (green). Nuclei are stained and colored blue. Scale bar, 20 um.

FIG. 25A shows a schematic of one embodiment of an assembled open -top chip microfluidic device 1700, showing open-top chambers 1763 and 1764 each located above a circular lower fluidic channel, e.g. 1751. Each chamber is surrounded by a deformable surface 1745 (e.g. membrane); spiral microchannels 1751 each are in fluidic communication with an inlet port 1719 located adjacent to an outlet port and an outlet port 1722 adjacent to an inlet port. Optionally a first vacuum port 1730; optionally a second vacuum port 1732, each vacuum port 1730 and 1732 connected to a first vacuum chamber 1737 or a second vacuum chamber 1738.

FIG. 25B shows a schematic of one embodiment of an exploded view of the embodiment depicted FIG. 25A shows an open-top chip device 1800, wherein a membrane 1840 resides between the bottom surface of the first chamber 1863 and the second chamber 1864 and spiral microchannels 1851.

FIG. 26A shows a schematic of one-embodiment (top view) of chip 1800 with a single chamber showing one embodiment of lower channel 1851 (left) and a combined view of an upper (blue) and lower channel (red). Black dots represent inlet and outlet ports.

FIG. 26B Illustrates an exploded (layer by layer) view of one-embodiment of an open top device as shown in FIG. 25A, showing membrane 1840 in between a chamber (blue) and the bottom channel (red).

FIG. 26C shows an exemplary schematic of one embodiment of a 3D Alveolus Lung On-Chip as an open top microfluidic chip demonstrating an air layer on top of an epithelial layer, e.g., alveolar epithelium layer or airway cell layer, overlaying a stromal area, e.g., including fibroblast cells, in an upper chamber/channel with microvascular endothelial cells, as one example of endothelial cells, in a lower channel, e.g. showing a cut away view of multiple areas (rectangles) as part of one spiral channel (red). Left: showing location of air-liquid interface (ALI) and membrane 1840 with a top closed on an open top chip. Right: showing chamber walls—blue; growth chamber—yellow and vascular circular channel cut-put views—red with a top partially opened.

FIG. 26D shows a photograph of one embodiment of an actual open top chip, cm scale on the left, actual chip in the middle with one view showing an overlay of an upper channel (blue) and lower channel (red), with respect to a US Penny for size.

### DETAILED DESCRIPTION OF THE INVENTION

The present invention is related to high-content microscopy imaging of microfluidic cell culture systems. A method of high-content microfluidic device microscopy is contemplated, along with related statistical analysis and microfluidic device adaptors.

Described herein is a novel end-to-end, automated workflow to capture and analyze confocal images of microfluidic devices containing multi-cellular organ culture in order to assess detailed cellular phenotype across large batches of microfluidic devices. By automating this process, not only is acquisition time reduced, but process variability and user bias is also minimized. Automation has enabled establishment, for the first time, of a framework of statistical best practice for microfluidic device imaging, creating the capability of using microfluidic devices and imaging for routine testing in drug discovery applications that rely on quantitative image data for decision making. The approach was tested using test compounds, such as compounds whose mechanism of toxicity was linked to mitochondrial damage with subsequent induction of apoptosis and necrosis, e.g, staurosporine, a tool inducer of apoptosis. The workflow has also been applied to assess the hepatotoxic effect of active drug candidates illustrating its applicability in drug safety assessment beyond testing tool compounds. Finally, it has been demonstrated that this approach could be adapted to microfluidic devices of different shapes and sizes through application to a microfluidic device seeded with kidney cells.

Presented herein is an invention consisting of a microfluidic device adapter, a high-content imaging workflow, and a method of statistical analysis for use with microfluidic devices. It is not intended that the present invention be limited by the type of microscope, microfluidic device, or cause for microfluidic device imaging (such as microfluidic device inspection, cellular experiments, bacterial experiments, organism experiments, chemical experiments, diagnostic experiments, etc.) In one embodiment, where the microfluidic device is seeded with cells, it is not intended that the present invention be limited by the cell type, cell density, etc. The high-content imaging workflow and statistical analysis presented herein may be used to investigate any microfluidic device. The high-content imaging workflow and statistical analysis presented herein are advantageous as they may be implemented across multiple industries that use microfluidic devices and may be used to investigate anything contained within a microfluidic device. The high-content imaging workflow presented herein is also advantageous as it has the potential to vastly increase the efficiency of microfluidic experiments, reduce image variability, improve image quality, and remove user bias.

One aspect of the invention presented herein is a high-content imaging workflow that has the capability of reducing acquisition time of microfluidic devices by as much as 95%, reducing imaging variability between microfluidic devices to less than 10%, improving imaging quality and removing user bias. The high-content imaging workflow may be used with any microfluidic device on any microscope that comprises a camera. Many microscopes are envisioned, such as confocal or light microscopes capable of imaging.

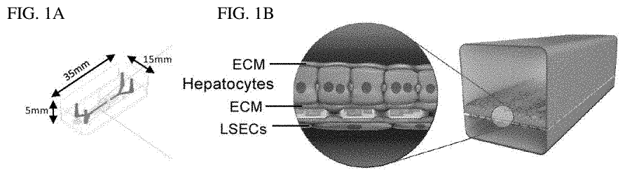

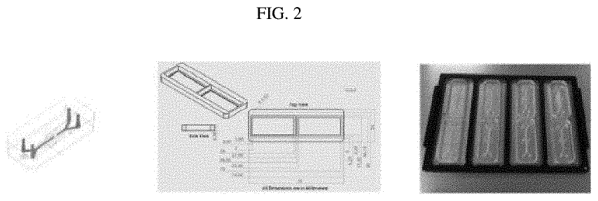

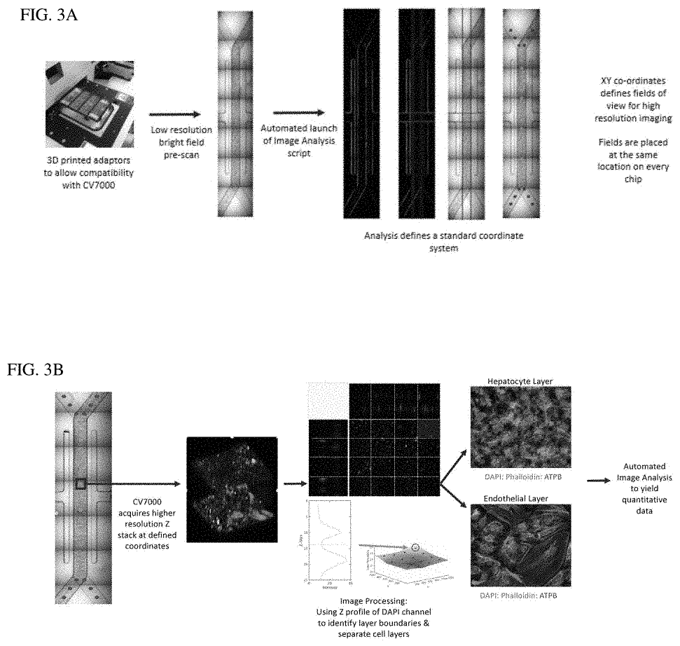

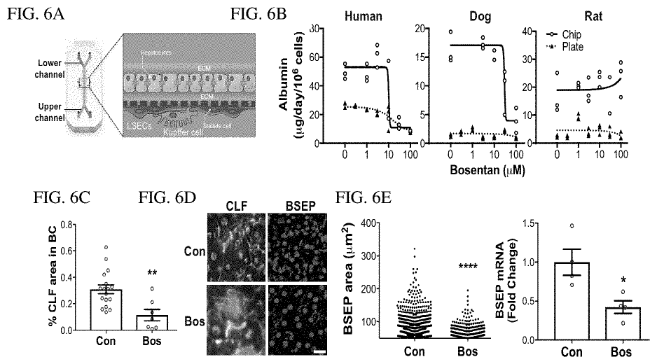

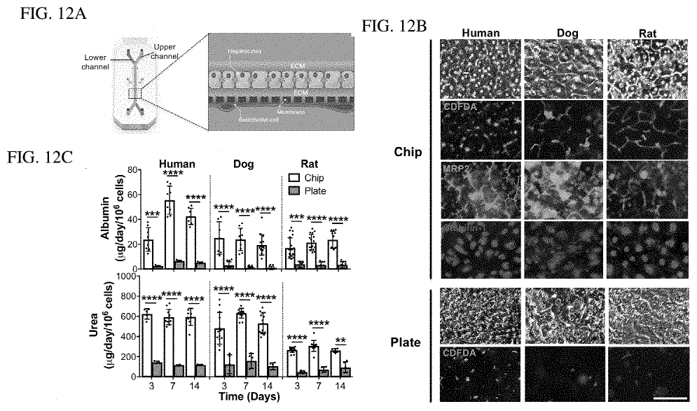

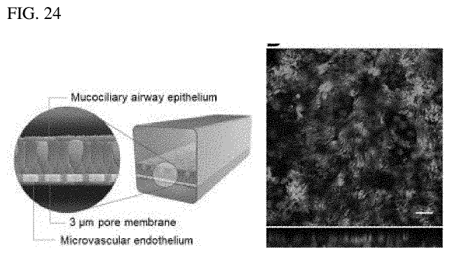

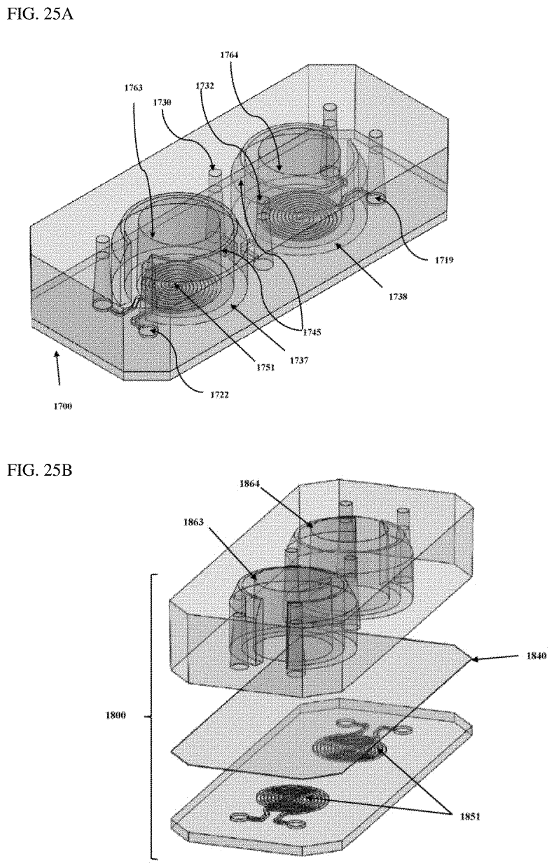

The microfluidic device adaptor, high-content imaging workflow and statistical analysis presented herein may be used with any microfluidic device. FIG. 1A, FIG. 1B. FIG. 2, FIG. 3A, FIG. 3B, FIG. 6A, FIG. 12A, FIG. 24, FIG. 25A-B, 26A-D, shows non-limiting examples of a microfluidic device that may be imaged using the high-content imaging workflow presented herein. The adaptor, high-content imaging workflow, and statistical analysis may even be used to image and analyze empty microfluidic devices. Imaging empty microfluidic devices may be useful in analyzing microfluidic device architecture and collecting information on, for example, fabrication variability. As such, in one embodiment, the microfluidic device contains nothing. Further, in one embodiment, the invention presented herein may be used to image and analyze microfluidic device architecture. In one embodiment, the microfluidic device is seeded with cells. In one embodiment, cells are prepared prior to being put in the microfluidic device. Any type of cell is considered, including, but not limited to, liver cells, kidney cells, brain cells, blood-brain-barrier cells, heart cells, skin cells, gut cells, spinal cord cells, lymph node cells, etc. Any cell source is considered, including primary isolates, or permanent cell lines, or a combination of the two. Furthermore, a variety of cell species are considered, including human, mouse, dog, monkey, etc. In one embodiment, the high-content imaging workflow presented herein may be used to image and analyze biological parameters, such as cell morphology, cell junctions, canalicular channels, lipid accumulation, alpha-smooth muscle actin (α-SMA), etc. In one embodiment, the high-content imaging workflow presented herein may be used to image bile canalicular networks. In one embodiment, the high-content imaging workflow presented herein may be used to study parameters (size, width, height, length, etc.) of bile canalicular networks. In one embodiment, the high-content imaging workflow presented herein may be used to image bile canalicular networks to investigate the health of canaliculi. In one embodiment, the high-content imaging workflow presented herein may be used to investigate whether canaliculi in microfluidic devices are interlocking. Healthy canaliculi tend to interlock.

The high-content imaging workflow presented herein may be especially helpful in imaging cells stained for α-SMA. Cells stained for α-SMA have a non-zero baseline when fluorescently imaging, and therefore it is difficult to distinguish between background and α-SMA related fluorescence. The high-content imaging workflow and related statistical analysis may be able to differentiate between relevant α-SMA fluorescence and background brightness. The same may be said for lipid accumulation imaging. Cells stained for lipid accumulation present a non-zero fluorescence base-line, and are therefore typically difficult to image. The high-content imaging workflow presented herein lends itself to distinguishing between relevant lipid accumulation fluorescence and background brightness.

In one embodiment, the microfluidic device is used to study chemical reactions. In one embodiment, the microfluidic device houses small specimen, such as bacteria, etc. In one embodiment, the microfluidic device may be seeded with cells. FIG. 1B shows how the microfluidic device in FIG. 1A may be seeded with cells. Microfluidic devices to be used with the high-content imaging workflow may, in some embodiments, comprise multiple channels, such as an upper channel (2-blue) and a lower channel (3-red). Microfluidic devices to be used with the high-content imaging workflow may also comprise architectural features such as the porous membrane (7-grey) seen in FIG. 1B. The microfluidic device shown in FIGS. 1A and 1B may comprise multiple cell layers, such a first cell layer (6-hepatocytes) and a second cell layer (5-endothelial cells), or aggregates of cells seeded into channels or chambers, such as organoids, etc. Cell layers may comprise endothelial and epithelial cell layers. Cell layers can be confluent, largely confluent, or comprise spacing between cells (i.e. patchy cell layers still may be considered cell layers).

In one embodiment, a seeding protocol may be followed to seed cells in a microfluidic device. The microfluidic device may also be coated, such as with an extracellular matrix (ECM) (4-grey). It is not intended that specific protocols presented herein limit the present invention. In the embodiment in which cells are seeded into the microfluidic device, any protocol may be used to seed the cells in the microfluidic device. One skilled in the art may determine an appropriate seeding protocol of cells into the microfluidic device to suit their individual needs.

In one embodiment, the following protocol may be followed to seed cells into a microfluidic device:
1. Coat the channels of the microfluidic device with extracellular matrix (4), wherein the microfluidic device's channels may have been previously functionalized, e.g. coated with Sulfo-SANPAH as described herein, and incubate overnight, such as at a temperature of 37° C. and a 95%/5% ratio of air to CO₂ or at room temperature.
2. Seed a first variety of cells in a first channel, such as a channel above a membrane, at a desired density in a cell culture media.
3. Optionally overlay the first monolayer with a gel.
4. Seed either the first or a second variety of cells in a second channel, such as the channel below a membrane, at a desired density in a cell culture media.
5. Optionally invert the microfluidic devices for an amount of time, nonlimiting examples include up to 3 hours, up to 4 hours.
6. Connect the microfluidic device to a pump, such as a pneumatic pump, in order to provide option flow.

In one embodiment, the cells in the microfluidic device may be tested with a compound or agent. In one embodiment, the cells in the microfluidic device may be tested with a pharmaceutical, cosmetic, food, chemical, etc. In one embodiment, the cells in the microfluidic device may be tested with an unapproved candidate compound in order to assess efficacy. In one embodiment, the following protocol may be followed to seed liver cells into a microfluidic device:
1. Coat the channels of the microfluidic device with extracellular matrix wherein the microfluidic device's channels may have been previously functionalized, e.g. coated with Sulfo-SANPAH as described herein, and incubate overnight, such as at a temperature of 37° C. and a 95%/5% ratio of air to CO₂.
2. Seed liver cells, such as hepatocytes, in a first channel, such as a channel above a membrane, at a desired density, such as 3.5×10⁶ cells/mL.

In some embodiments, hepatocytes may be seeded in Complete Hepatocyte Seeding Media: Including Base Hepatocyte Seeding Medium: William's E Medium (WEM)+ (with phenol red), L-GlutaMAX™ and Pen/strep with the addition of ITS+ Premix (1%), Corning, Ascorbic Acid (0.05 mg/mL), Sigma, Dexamethasone (1 μM), and 5% FBS.
3. Overlay the hepatocyte monolayer with a Matrigel Matrix.

In some embodiments, a Hepatocyte Overlay Medium is used: Complete Hepatocyte Maintenance Medium with 0.25 mg/mL Matrigel®.
4. In some embodiments, seed liver cells, such as sinusoidal endothelial cells, in a second channel, such as the channel below a membrane, at a desired density, such as 2 to 4×10⁶ cells/mL in a medium, such as an endothelial media, such as CSC media.

When the LSECs are not as proliferative as expected, the concentration can be increased from up to 12×10⁶ cells/mL (3 times the final seeding concentration), in order to achieve a confluent monolayer within the channel. In some embodiments, LSECs are adjusted to a density of 9×10⁶ cells/mL (3 times the final seeding concentration) prior to combining with stellate and Kupffer cells to generate a bottom channel tri-cell mixture.

In some embodiments, stellate cells are adjusted to a density of 0.3×10⁶ cells/mL (3 times the final seeding concentration) prior to combining with LSECs and Kupffer cells to generate the bottom channel tri-cell mixture.

In some embodiments, Kupffer cells are adjusted to a density of 3×10⁶ cells/mL prior to combining with LSECs and stellate cells to generate the bottom channel tri-cell mixture.

In some embodiments, nonparenchymal cells (NPCs) are seeded in NPC Seeding Medium: a 1:1 mixture of Complete Hepatocyte Maintenance Medium, omitting dexamethasone: Base LSEC Culture Medium, with 10% FBS.
5. Optionally invert the microfluidic devices for an amount of time, such as two hours, or up to 4 hours, in order for attachment to the membrane. In one embodiment, inverting the microfluidic device allows the cell attachment to the membrane.
6. Connect the microfluidic device to a pump, such as a pneumatic pump.
7. Flow media through one or more of the channels within the microfluidic device at a flow rate, such as 30 μL/hour.

In some embodiments, hepatocytes are cultured over time in Complete Hepatocyte Maintenance Medium: Including Hepatocyte Maintenance Media: WEM—(without phenol red), 1% Pen/Strep, 1% L-GlutaMAX™ with the addition of ITS+ Premix (1%), Corning, Ascorbic Acid (0.05 mg/mL), Sigma, and Dexamethasone (1 μM).

In some embodiments, nonparenchymal cells are cultured over time in NPC Maintenance Medium: a 1:1 mixtures of Complete Hepatocyte Maintenance Medium omitting dexamethasone: Base LSEC Culture Medium, with 2% FBS.

In one embodiment, the liver cells in the microfluidic device may be tested with a compound or agent. In one embodiment, the cells in the microfluidic device may be tested with a pharmaceutical, cosmetic, food, chemical, etc. In one embodiment, the liver cells in the microfluidic device may be tested with an unapproved candidate compound in order to assess efficacy.

In one embodiment, the following protocol may be followed to seed kidney cells into a microfluidic device(s):
1. Coat the channels of the microfluidic device(s) with an extracellular matrix, such as a collagen I matrix and/or a human collagen IV, wherein the microfluidic device's channels may have been previously functionalized, e.g. coated with Sulfo-SANPAH as described herein, and incubate overnight, such as at room temperature.
2. Seed kidney cells, such as proximal tubule epithelial, into the channels of a microfluidic device(s) at a desired density, such as 1.0×10⁶ cells/mL, or 5 μL per microfluidic device.
3. Place the microfluidic device(s) in an incubator, such as with a 95%/5% ratio of air to CO₂ and temperature of 37° C., for an amount of time, such as 24 hours.
4. Connect the microfluidic device to a pump, such as a perfusion platform.
5. Flow media through one or more of the channels within the microfluidic device at a flow rate, such as 0.5 μL/hour.

In one embodiment, the kidney cells in the microfluidic device may be tested with a compound or agent. In one embodiment, the cells in the microfluidic device may be tested with a pharmaceutical, cosmetic, food, chemical, etc. In one embodiment, the kidney cells in the microfluidic device may be tested with an unapproved candidate compound in order to assess efficacy.

In one embodiment, microfluidic devices are placed in a microfluidic device adaptor in order to interface with a microscope. It is not intended that the invention presented herein is limited to the type of microscope. In one embodiment, the microscope is a confocal microscope. Confocal microscopes tend to be designed to interface with cell culture plates. In one embodiment, the microfluidic device adaptor is compatible with microscopes designed to interface with cell culture plates. It is not intended that the present microfluidic device adaptor be limited by the type of microfluidic device, material, shape, or number of microfluidic devices it is able to hold. In one embodiment, the microfluidic device adaptor holds a single microfluidic device. In one embodiment, the microfluidic device adaptor holds multiple (2, 3, 4, etc.) microfluidic devices. In embodiment, the microfluidic device adaptor holds a plurality of microfluidic devices, such as 2, 3, 4, etc. In one embodiment the microfluidic device adaptor is plastic. In one embodiment, the microfluidic device adaptor is 3D printed. In one embodiment, the microfluidic device adaptor is injection molded. In one embodiment, the microfluidic device is machined from metal. In one embodiment, the microfluidic device adaptor is metal. FIG. 2 shows one embodiment of a microfluidic device adaptor (9) for a microfluidic device (1) to be seeded with liver cells. In one embodiment, the microfluidic device adaptor comprises a substrate and alignment features. In one embodiment, the microfluidic device adaptor comprises a substrate and alignment features cut into the substrate. In one embodiment, the microfluidic devices fit into the alignment features, such as with a compression fit. In one embodiment, the microfluidic devices fit into the microfluidic device adaptor such as with a compression fit due to radial compression. In one embodiment, the microfluidic device adaptor comprises a substrate with microfluidic device shaped holes cut through it. In one embodiment, the microfluidic devices fit into the holes, such as with a compression fit. In one embodiment, the microfluidic devices fit into the microfluidic device adaptor such as with a compression fit due to radial compression. In one embodiment, the microfluidic device adaptor comprises a substrate and clips configured for holding microfluidic devices in place. In one embodiment, the microfluidic device adaptor is configured for microfluidic devices seeded with liver cells. In one embodiment, the microfluidic device adaptor is configured for microfluidic devices seeded with kidney cells. The microfluidic device adaptor may be used with any microfluidic device.

The general approach of identifying landmarks at low magnification can be applied to any microfluidic device architecture. Intelligent, high-content scanning, otherwise known as high-content, of microfluidic devices from different manufacturers has been conducted in order to illustrate that the method may be applied across different microfluidic device architectures. To generalize the approach for any microfluidic device system, one may apply the following embodiment, consisting of three steps. First, a round of low-resolution images are taken in order to create a reference image set. Second, coordinates of fields of interest are placed manually on a reference image set. Third, for the intelligent scanning run, the first pass images are aligned to the reference image using a rigid registration algorithm, to determine where to place the field coordinates on the first pass images. In one embodiment, to account for brightness and focus variations in the bright field images, both reference and test images may be normalized, smoothed and edge filtered, before performing the registration.

In one embodiment, to define the same field of view, abbreviated as FOV, or focal height on every microfluidic device, a common coordinate system may be defined, based on features which are present in each microfluidic device. In one embodiment, this coordinate system may be referenced on the first set of acquisitions in order to direct the second set of acquisitions. In one embodiment, the image analysis identifies a coordinate system based upon microfluidic device architecture. In one embodiment, the microfluidic device architecture consists of microfluidic channel walls. In one embodiment, the image analysis identifies a coordinate system based upon microfluidic device architecture. In one embodiment, the microfluidic device architecture consists of a membrane. In one embodiment, the coordinate system's location is based on pores in the membrane. In one embodiment, the coordinate system's location based on the location of a first surface of the membrane. In one embodiment, the coordinate system's location is based on the location of a second surface of the membrane. In one embodiment, the coordinate system's location is located based on a first and second surface of the membrane. In one embodiment, the image analysis identifies a coordinate system based upon microfluidic device architecture. In one embodiment, the microfluidic device architecture consists of microchannel inlet or outlet ports. In one embodiment, the image analysis identifies a coordinate system based upon microfluidic device architecture. In one embodiment, the microfluidic device architecture consists of tissue culture anchors, such as for skeletal muscle tissue.

In one embodiment, microscope images are corrected for variation in illumination across each image, then stitched together to form one image covering the whole of the microfluidic device area. In one embodiment, a Hessian-based trough detection was then applied to enhance dark lines in the image, followed by a Radon transform to detect straight lines in the images, as seen in FIG. 3A. In one embodiment, microfluidic device architecture used to define the coordinate system is the location of the microchannel walls. In one embodiment, the edges of the microfluidic device microchannels are detected as lines close to the vertical orientation and with an expected separation, defining the horizontal location of the main channel. In one embodiment, lines, such as channel walls, define the standard coordinate system for a type of microfluidic device. In one embodiment, coordinates equally spaced along the center of the main channel are chosen, with a gap marginally larger than the size of the FOV. In one embodiment, points defined in these coordinates will lie at the same position on every microfluidic device.

Coordinate systems may be identified, in one embodiment, based on cells within the microfluidic device. It is not intended that the image analysis and coordinate system identification be limited by the cell type, cell size, cell density, cell age, cell culture length, whether the cell is attached or not to a surface, cell location, etc. Coordinate systems may be identified based on cells of different types, sizes, densities, ages, culture levels, attachment levels, cell location, etc. In one embodiment, cells are cultured on to a surface of the microfluidic device, such as channel walls or membranes, such that they are attached to said surface. In another embodiment, the cells are not attached to any surface, such as channel walls or membranes. The coordinate system may be a targeted cellular microsystem, either two or three dimensional. In one embodiment, the coordinate system is a recapitulated physiological system, such as a tract, vessels, stratified cellular structures, etc.

Geometric criteria used to identify coordinate systems may be based on the location of cells on or within a microfluidic device. In one embodiment, the image analysis identifies the proximity of cells to each other or features of the microfluidic device in order to identify a coordinate system for a second set of acquisitions. The identification of the location of cells within the microfluidic device may be used regardless of cell attachment or not. In one embodiment, it is desired to image cells of a particular location. In this embodiment, the image analysis of the first set of acquisitions detects cells of that particular location, sets a coordinate system about them, and conducts a second set of acquisitions.

Geometric criteria used to identify coordinate systems may be based on cell geometry or shape. Cell shape factors include cell circularity, eccentricity and solidity. Cell shape may be identified in order to then identify a coordinate system for a second set of acquisitions. Cell circularity is the amount the cell is shaped as a circle. A circle has a circularity of one. Circularity is also known as isoperimetric quotient Cell eccentricity is how much a cell deviates from being circular. The eccentricity of a circle is zero. Oftentimes non-attached cells exhibit more circular shapes, while attached cells exhibit more elongated shapes. Cell solidity, also known as convexity, is the proportion of the cell that fits within a smooth line around the cell. A cell with many protrusions or indentations would have a cell solidity closer to zero than a cell with smooth edges. Other geometric criteria or shape factors that may be used to identify a coordinate system within a microfluidic device are aspect ratio, elongation, compactness, waviness, etc. In one embodiment, it is desired to image cells of a particular shape. In this embodiment, the image analysis of the first set of acquisitions detects cells of that particular shape, sets a coordinate system about them, and conducts a second set of acquisitions. In one embodiment, the image analysis identifies the size of cells in order to then identify a coordinate system. In one embodiment, it is desired to image cells of a particular size. In this embodiment, the image analysis of the first set of acquisitions detects cells of that particular size, sets a coordinate system about them, and conducts a second set of acquisitions.

In one example, cells may be seeded in a microfluidic device in such a way as to recapitulate a biliary canaliculus. In such an embodiment, the coordinate system is a biliary canaliculus. In one embodiment, cell size smaller than 70 μm² and greater than 7 μm², may be used to determine the coordinate system of a biliary canaliculi. In one embodiment, the high-content imaging workflow may detect cell solidity greater than 0.7 in order to determine the coordinate system of a biliary canaliculi. In some cases, jagged, elongated canaliculi are sought during the first round of acquisitions. In some embodiments, the high-content imaging workflow may detect circularity below 0.5 in order to determine the coordinate system of a biliary canaliculi. In some embodiments, the high-content imaging workflow may detect eccentricity greater than 0.8 in order to determine the coordinate system of a biliary canaliculi.

Geometric criteria of cells may be compared to known geometric criteria of other objects or object surfaces, including features of a microfluidic device. In one embodiment, the circularity of cells may be compared to the circularity of foreign objects, such as round, synthetic beads. In one embodiment, the size of cells may be compared to the size of foreign objects, such as synthetic beads. Further, geometric criteria, such as cell shape, may be used to gauge cell health. Again, it is not intended that the high-content imaging workflow, or any part of the invention presented herein, be limited by the microfluidic device architecture or geometric criteria chosen for image analysis.

One embodiment of the invention presented herein is a method of imaging microfluidic devices comprising: (a) providing a microfluidic device comprising a membrane, said membrane separating two microfluidic channels; (b) providing a microscope capable of image acquisition; (c) taking a first set of microscopic acquisitions; (d) analyzing the first set of microscope acquisitions, determining a focal height and locating a standard coordinate system, wherein the coordinate system is located based on the location of the membrane within the microfluidic device; (e) and taking a second set of microscopic acquisitions based on the coordinate system located in the first set of microscopic acquisitions. One embodiment of the invention presented herein is a method of imaging microfluidic devices comprising: (a) providing a microfluidic device comprising microchannels, said microchannels comprising channel walls; (b) providing a microscope capable of image acquisition; (c) taking a first set of microscopic acquisitions; (d) analyzing the first set of microscope acquisitions, determining a focal height and locating a standard coordinate system, wherein the coordinate system is located based on the location of the membrane within the microfluidic device; (e) and taking a second set of microscopic acquisitions based on the coordinate system located in the first set of microscopic acquisitions. In one embodiment, the microscope is a confocal microscope.

In one embodiment, cells are contained within the microfluidic device. In one embodiment, the cells contained within the microfluidic devices are fluorescently active. In one embodiment, the cells are naturally fluorescently active. In one embodiment, the cells have been modified to be fluorescently active. In one embodiment, the cells have been genetically altered to be fluorescently active. In one embodiment, a fluorescent dye is added to the cells. In one embodiment, the cells fluoresce differently when not in contact and in contact with compounds. In one embodiment, the cells are fluorescently tagged facilitating the detection of a biomolecule, such as a protein, antibody, amino acid, etc.

In one embodiment, the microfluidic devices are imaged on a confocal microscope. In one embodiment, the microfluidic devices are excited with one or more excitation lasers. It is not intended that the invention presented herein be limited by the type of excitation laser. In one embodiment, the excitation laser is a 405 nm excitation laser. In one embodiment, the excitation laser is a 405 nm excitation laser with a 445/45 nm band pass emission filter. In one embodiment, the excitation laser is a 561 nm excitation laser. In one embodiment, the excitation laser is a 561 nm excitation laser with a 600/37 nm band pass emission filter. In one embodiment, the excitation laser is a 488 nm excitation laser. In one embodiment, the excitation laser is a 488 nm excitation laser with a 525/50 nm band pass emission filter. In one embodiment, the excitation laser is a 640 nm excitation laser. In one embodiment, the excitation laser is a 640 nm excitation laser with a 767/37 nm band pass emission filter.

In one embodiment, the microfluidic devices are imaged over a range at intervals to cover a plurality of cell monolayers. It is not intended that the present invention be limited by the exact size of the imaging range. In one embodiment, the microfluidic devices are imaged over a 120 μm range at 5 μm Z intervals to cover a plurality of cell monolayers. In one embodiment, the microfluidic devices are imaged over a 120 μm range at 1 μm Z intervals to cover a plurality of cell monolayers. In one embodiment, the microfluidic devices seeded with liver cells are imaged over a 120 μm range at 5 μm Z intervals to cover a plurality of cell monolayers. In one embodiment, the microfluidic devices seeded with kidney cells are imaged over a 120 μm range at 1 μm Z intervals to cover a plurality of cell monolayers.

One embodiment of the invention is a method to perform intelligent, high-content imaging of microfluidic devices on a microscope. In one embodiment, the microscope is a confocal microscope. In one embodiment, the microscope is a light microscope. In one embodiment of the invention, a first set of microscope acquisitions takes places, followed by image analysis, followed by a second set of microscope acquisitions based off the analysis of the first round of imaging. In one embodiment, the first set of microscope acquisitions are low-resolution. In one embodiment, the second set of imaging is high-resolution. In one embodiment, the first set of microscope acquisitions are taken using a bright field. In one embodiment, the second set of microscope acquisitions are taken using laser excitation. In one embodiment, the first set of microscope acquisitions are taken as bright-field images and are acquired using a 4× objective lens using a 100 W Halogen lamp as an illumination source. In one embodiment, the second set of microscope acquisitions are taken using excitation lasers. In one embodiment, both series of microscope acquisitions are taken using bright field. In one embodiment, both series of microscope acquisitions are taken using laser excitation.

In one embodiment, the fluorescent images are acquired as a single stack comprising a first cell monolayer (5) and a second cell monolayer (6) together, separated by a membrane (7). In one embodiment, the fluorescent images are acquired as a single stack comprising the endothelial layer and hepatocyte layer together, separated by the membrane (7). In the same embodiment, the stack is separated into two layers by finding the minimum in the Hoechst channel along the z-direction. To account for variations in layer locations across the image the image may be broken down into 4×4 sub regions, the height of the minimum is located in each region and then this height may be interpolated over the whole image, giving a surface, which separated the two layers as shown in FIG. 3B. For morphological measurements of cells and nuclei may be segmented in three dimensions, and measures of cell number and organization may be computed. For fluorescence measurements, a maximum projection may be calculated for each layer, and cell regions are segmented using the phalloidin channel, starting from nuclear regions as seeds, to calculated single cell intensity values. Where appropriate, the fluorescence images may be separated into distinct length scales of signal using morphological opening operations.

In one embodiment, microfluidic devices seeded with cells, such as organ cells (liver, kidney, brain, lymph node, gut, skin, skeletal muscle, etc.) may be imaged using the high-content techniques presented herein. In one embodiment, the high-content imaging techniques presented herein allows cellular phenotypes, such as morphology, proliferation, apoptosis, and mitochondrial structure to be captured across many microfluidic devices much more efficiently than typical microfluidic device imaging. Maximizing microfluidic device imaging efficiency is advantageous, as it decreases variability, increases economy and time savings, and also allows for larger experiments, increasing the potential findings of scientific experiments. Typically conducting imaging of microfluidic devices in order to investigate biological parameters, such as cell morphology, cell junction strength, marker quantification, etc. can take hours per microfluidic device. The high-content imaging workflow presented herein has the capability to image a microfluidic device in as little as five to ten minutes. In a preferred embodiment of the inventions presented herein, the imaging of eight microfluidic devices seeded with cells may be decreased from 16 hours to just 50 minutes, for a time saving of 95%.

The microfluidic device seeded with liver cells described herein have been designed to recapitulate in vivo liver function in one embodiment of their use. As an example, the microfluidic devices have been shown to display physiologically relevant levels of albumin and urea secretion as well as metabolic competency for at least 14 days. The high-content imaging workflow presented herein may be used at any point in cell culture. In one embodiment, the high-content imaging workflow may be used immediately after cell seeding. In one embodiment, the high-content imaging workflow may be used hours after cell seeding. In one embodiment, the high-content imaging workflow may be used days after cell seeding. In one embodiment, the high-content imaging workflow may be used after at least 14 days after cell seeding. Although biomarkers of cellular injury in response to drug exposure can be measured, it has been demonstrated here that high-content imaging offers a complimentary approach of capturing cell phenotype changes in response to a hepatotoxic compound, thereby enhancing the application of these systems for mechanistic studies. Image analysis confirmed staurosporine-induced apoptosis with marked increases in CC3 levels. Morphology, proliferation and apoptosis endpoints have been described as examples, but with the rapid advancement of sophisticated image analysis algorithms and use of Machine Learning, the potential phenotypes that could be analyzed can extend to any available antibodies and markers. Moreover, this approach is not limited by the small number of cells within a microfluidic device. By imaging in three dimensions the cell layers may be separated, and the cellular phenotype may be read at the single cell level, thereby allowing quantification of heterogeneity, for example in the phalloidin intensity, and an understanding of phenotype which would not be apparent in averaged population measurements.

A framework of statistical best practice for microfluidic devices studies has been built. First principles of optimal experimental design were applied to randomize microfluidic devices imaging location and order. This removed all bias associated with the imaging workflow. A Bayesian analysis was then used to understand the variability in the data and could decouple variability due to technical error from experimental conditions.

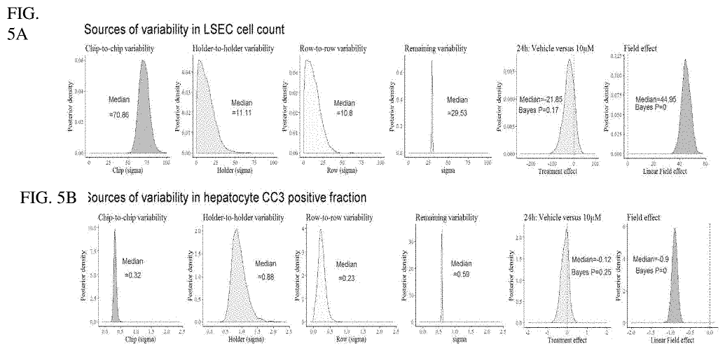

FIGS. 5A-5B show Bayesian analysis of a test compound to understand the variability in the data allowing the decoupling of the variability due to technical error (in blue) from experimental conditions (in green). Two examples are given, as shown in FIG. 5A and FIG. 5B. FIG. 5A shows analysis based on LSEC cell count. FIG. 5B shows analysis based on CC3 (Cleaved Caspase 3) signal in hepatocytes.

Finally, a power analysis was run to identify the minimum sample size necessary to detect a given effect size. This best practice standard guides the experimental design and analysis of microfluidic device studies. The application of microfluidic devices allows the extraction of robust data from these complex model systems. In one embodiment, a framework of statistical best practice for microfluidic device studies to reduce bias and variability has been developed. In one embodiment, a statistical method of analyzing microfluidic device acquisitions in order to decouple sources of variability is considered, comprising: (a) randomizing the order in which microfluidic devices are imaged; (b) fitting a Bayesian linear regression model to the images; (c) estimating treatment effects, time effects, and microfluidic device variability based on the Bayesian linear regression; and (d) reporting Bayes p-values in order to verify statistical significance. In one embodiment, a Bayes p-value of less than 0.025 is accepted as statistically significant.

Here described is the first setup of an end-to-end, automated workflow to capture and analyze confocal images of a multicellular microfluidic devices model. This process allows detailed cellular phenotypes (such as morphology, proliferation, apoptosis, and mitochondrial structure) to be captured across large batches of microfluidic devices. By automating this process, reduced acquisition and user interaction time has been reduced. Prior to this, three-dimensional confocal imaging of the microfluidic devices, such as microfluidic devices seeded with liver or kidney cells, was done in a manual fashion (i.e. manually locating and defining FOV) and took more than two hours per microfluidic device. Using the high-content imaging workflow presented herein, the imaging of a microfluidic device, in an exemplary embodiment, can be done in about five minutes. Whilst the capacity for high throughput screening may not be required, a single study to evaluate hepatoxicity of a test compound in a dose dependent manner, across multiple donors typically requires analysis of upwards of 50 microfluidic devices. Even at this scale, manual acquisition of images would take weeks and could also suffer from variability. In summary, an end-to-end, automated workflow to acquire and analyze confocal images of multicellular microfluidic devices of different formats to probe cellular phenotype across large batches of microfluidic devices has been established. By automating this process, acquisition time, process variability and user bias can be reduced. Taken together, this has enabled an establishment of a unique framework of statistical best practice for microfluidic devices imaging that will enhance the data that may be obtained from these model systems going forward and enhance their utility and applications. The establishment of the best practice framework described here may contribute to the growing platform of evidence that shows these micro-engineered systems accurately recapitulate in vivo functionality. In addition, the framework can play a role in the development of methodology guidelines that assess the reproducibility, robustness and clinical translatability of microfluidic devices.

Exemplary embodiments are presented below in order to elucidate model uses of the inventions presented herein. One exemplary embodiment of the present invention is a method of imaging microfluidic devices comprising: (a) providing a microfluidic device comprising a membrane, said membrane separating two microfluidic channels; (b) providing a microscope capable of image acquisition; (c) taking a first set of microscopic acquisitions; (d) analyzing the first set of microscope acquisitions, determining a focal height and locating a standard coordinate system, wherein the coordinate system is located based on the location of the membrane within the microfluidic device; (e) and taking a second set of microscopic acquisitions based on the coordinate system located in the first set of microscopic acquisitions.

In an exemplary embodiment, the microscope may be a confocal microscope. In one embodiment, the first set of microscopic acquisitions are low-resolution. In one embodiment, the second set of microscopic acquisitions are high-resolution. In one exemplary embodiment, the microfluidic device is seeded with cells. In one embodiment, the second set of microscope acquisitions are used to evaluate the effect of an agent on the cells. In one embodiment, the agent is a pharmaceutical. In one embodiment, the cells are cultured for more than seven days. In one embodiment, the cells are located in the channels separated by the membrane. In one embodiment, the second set of microscopic acquisitions comprises a three-dimensional acquisition. In one embodiment, the three-dimensional acquisition comprises an endothelial cell layer and hepatocyte cell layer together, separated by the membrane. In one embodiment, the cells are liver cells. In one embodiment, the liver cells are hepatocytes and sinusoidal endothelial cells. In one embodiment, the hepatocytes and sinusoidal endothelial cells are human hepatocytes and human sinusoidal endothelial cells. In one embodiment, the cells are kidney cells. In one embodiment, the microscopic acquisitions are of individual cells. In one embodiment, the channels of the microfluidic device are coated with a mixture of extracellular matrix. In one embodiment, the method further comprises applying flow to the channels. In one embodiment, the flow exerts shear stress on the cells. In one embodiment, the method further comprises identifying the presence of cells in the microfluidic device. In one embodiment, the method further comprises identifying the presence of nuclear stains on the cells in the microfluidic device. In one embodiment, the method further comprises identifying membrane markers between the cells in the microfluidic device. In one embodiment, the membrane markers are tight junction markers. In one embodiment, the tight junction markers are zonula occludens-1 (ZO-1) markers. In one embodiment, the tight junction markers are cadherin markers. In one embodiment, the cadherin markers are epithelial cadherin markers. In one embodiment, the method further comprises identifying the presence of a gradient along the length of a microfluidic device. In one embodiment, the gradients are identified downstream in the microfluidic device channels. In one embodiment, the gradients are identified upstream in the microfluidic device channels. In one embodiment, the gradient is a change in the number of metabolites. In one embodiment, the gradient is an oxygen gradient. In one embodiment, the gradient is a change in the number of nuclei present. In one embodiment, the method further comprises identifying the presence of α-SMA. In one embodiment, the method further comprises identifying lipid accumulation. In one embodiment, the method further comprises identifying biocanaliculi. In one embodiment, the cells are identified using geometric criteria. In one embodiment, the geometric criteria are selected from a list comprising of size, circularity, eccentricity and solidity.

One exemplary embodiment of the present invention is a method of imaging microfluidic devices comprising: (a) providing a microfluidic device comprising a membrane, said membrane separating two microfluidic channels; (b) providing a microscope capable of image acquisition; (c) taking a first set of microscopic acquisitions; (d) analyzing the first set of microscope acquisitions, determining a focal height and locating a standard coordinate system, wherein the coordinate system is located based on the location of the membrane within the microfluidic device; (e) and taking a second set of microscopic acquisitions based on the coordinate system located in the first set of microscopic acquisitions.

In one embodiment, the coordinate system is located based on pores in the membrane. In one embodiment, the coordinate system is located based on the location of a first surface of the membrane. In one embodiment, the coordinate system is located based on the location of a second surface of the membrane. In one embodiment, the coordinate system is located based on a first and second surface of the membrane. In one embodiment, the method further comprises identifying the presence of cells in the microfluidic device. In one embodiment, the method further comprises identifying the presence of nuclear stains on the cells in the microfluidic device. In one embodiment, the method further comprises identifying membrane markers between the cells in the microfluidic device. In one embodiment, wherein the membrane markers are tight junction markers. In one embodiment, the tight junction markers are zonula occludens-1 (ZO-1) markers. In one embodiment, wherein the tight junction markers are cadherin markers. In one embodiment, wherein the cadherin markers are epithelial cadherin markers. In one embodiment, the method further comprises identifying the presence of a gradient along the length of a microfluidic device. In one embodiment, wherein the gradients are identified downstream in the microfluidic device channels. In one embodiment, wherein the gradients are identified upstream in the microfluidic device channels. In one embodiment, wherein the gradient is a change in the number of metabolites. In one embodiment, wherein the gradient is an oxygen gradient. In one embodiment, wherein the gradient is a change in the number of nuclei present. In one embodiment, the method further comprises identifying the presence of α-SMA. In one embodiment, the method further comprises identifying lipid accumulation. In one embodiment, the method further comprises identifying biocanaliculi. In one embodiment, the cells are identified using geometric criteria. In one embodiment, the geometric criteria are selected from a list comprising of size, circularity, eccentricity and solidity. In one embodiment, the microscope is a confocal microscope. In one embodiment, the first set of microscopic acquisitions are low-resolution. In one embodiment, the second set of microscopic acquisitions are high-resolution. In one embodiment, the microfluidic device is seeded with cells. In one embodiment, the second set of microscope acquisitions are used to evaluate the effect of an agent on the cells. In one embodiment, the agent is a pharmaceutical. In one embodiment, the cells are cultured for more than seven days. In one embodiment, the cells are located in the channels separated by the membrane. In one embodiment, the second set of microscopic acquisitions comprises a three-dimensional acquisition. In one embodiment, the three-dimensional acquisition comprises an endothelial cell layer and hepatocyte cell layer together, separated by the membrane. In one embodiment, the cells are liver cells. In one embodiment, the liver cells are hepatocytes and sinusoidal endothelial cells. In one embodiment, the hepatocytes and sinusoidal endothelial cells are human hepatocytes and human sinusoidal endothelial cells. In one embodiment, the cells are kidney cells. In one embodiment, the microscopic acquisitions are of individual cells. In one embodiment, the channels of the microfluidic device are coated with a mixture of extracellular matrix. In one embodiment, the method further comprises applying flow to the channels. In one embodiment, the flow exerts shear stress on the cells.

An exemplary method of analyzing cellular phenotype changes following agent exposure comprises: (a) providing one or more microfluidic device comprising microchannels, said microchannels comprising microchannel walls; (b) providing a microscope capable of image acquisition; (c) treating a number of the microfluidic devices an agent and a number of microfluidic devices with a control media; (d) taking a first set of microscopic acquisitions; (e) analyzing the first set of microscope acquisitions and locating a standard coordinate system, wherein the coordinate system is located based on the location of the microchannel walls within the microfluidic device; (f) taking a second set of microscopic acquisitions based on the coordinate system located in the first set of microscopic acquisitions; (g) making endpoint measurements of the acquisitions; (h) fitting a Bayesian linear regression model to the measurements; (i) estimating a linear field effect based on the Bayesian linear regression; and (j) comparing the linear field effect from microfluidic devices treated with an agent versus microfluidic device treated with a control media.

In one embodiment, the microscope is a confocal microscope. In one embodiment, the first set of microscopic acquisitions are low-resolution. In one embodiment, the second set of microscopic acquisitions are high-resolution. In one embodiment, the microfluidic device is seeded with cells. In one embodiment, the second set of microscope acquisitions are used to evaluate the effect of an agent on the cells. In one embodiment, the agent is a pharmaceutical. In one embodiment, the cells are cultured for more than seven days. In one embodiment, the cells are located in the channels separated by the membrane. In one embodiment, the second set of microscopic acquisitions comprises a three-dimensional acquisition. In one embodiment, the three-dimensional acquisition comprises an endothelial cell layer and hepatocyte cell layer together, separated by the membrane. In one embodiment, the cells are liver cells. In one embodiment, the liver cells are hepatocytes and sinusoidal endothelial cells. In one embodiment, the hepatocytes and sinusoidal endothelial cells are human hepatocytes and human sinusoidal endothelial cells. In one embodiment, the cells are kidney cells. In one embodiment, the microscopic acquisitions are of individual cells. In one embodiment, the channels of the microfluidic device are coated with a mixture of extracellular matrix. In one embodiment, the method further comprises applying flow to the channels. In one embodiment, the flow exerts shear stress on the cells.

An exemplary method of analyzing cellular phenotype changes following agent exposure comprises: (a) providing one or more microfluidic device comprising a membrane, said membrane separating two microfluidic channels, each channel seeded with cells; (b) providing a microscope capable of image acquisition; (c) treating a number of the microfluidic devices an agent and a number of microfluidic devices with a control media; (d) taking a first set of microscopic acquisitions; (e) analyzing the first set of microscope acquisitions and locating a standard coordinate system, wherein the coordinate system is located based on the location of the membrane within the microfluidic device; (f) taking a second set of microscopic acquisitions based on the coordinate system located in the first set of microscopic acquisitions; (g) making endpoint measurements of the acquisitions; (h) fitting a Bayesian linear regression model to the measurements; (i) estimating a linear field effect based on the Bayesian linear regression; and (j) comparing the linear field effect from microfluidic devices treated with an agent verses microfluidic device treated with a control media.

In one embodiment, the microscope is a confocal microscope. In one embodiment, the first set of microscopic acquisitions are low-resolution. In one embodiment, the second set of microscopic acquisitions are high-resolution. In one embodiment, the microfluidic device is seeded with cells. In one embodiment, the second set of microscope acquisitions are used to evaluate the effect of an agent on the cells. In one embodiment, the agent is a pharmaceutical. In one embodiment, the cells are cultured for more than seven days. In one embodiment, the cells are located in the channels separated by the membrane. In one embodiment, the second set of microscopic acquisitions comprises a three-dimensional acquisition. In one embodiment, the three-dimensional acquisition comprises an endothelial cell layer and hepatocyte cell layer together, separated by the membrane. In one embodiment, the cells are liver cells. In one embodiment, the liver cells are hepatocytes and sinusoidal endothelial cells. In one embodiment, the hepatocytes and sinusoidal endothelial cells are human hepatocytes and human sinusoidal endothelial cells. In one embodiment, the cells are kidney cells. In one embodiment, the microscopic acquisitions are of individual cells. In one embodiment, the channels of the microfluidic device are coated with a mixture of extracellular matrix. In one embodiment, the method further comprises applying flow to the channels. In one embodiment, the flow exerts shear stress on the cells.

Several protocols are listed below as which are exemplary embodiments to be used with the high content imaging workflow presented herein.

### Standard Protocol for Bright-Field and Phase-Contrast Imaging Microfluidic Devices

Below is an example of an exemplary protocol for bright-field and phase-contrast imaging microfluidic devices. The following protocol is an exemplary protocol to be used in high-content imaging workflow.

The goal of the experiment is to image cells in microfluidic devices. Microfluidic devices include Emulate Organ Chips. Steps involve the assessment of cell morphology via bright-field microscopy and use of a phase-contrast condenser if better visualization of cellular structure is required. Materials required include a bright-field microscope and a phase-contrast condenser. In this example, the microfluidic device comprises a channel comprising a central membrane, a first channel on a first side of the membrane, and a second channel on the second side of the membrane. For the example below, the channels separated by the membrane may be oriented vertically, such that the first channel may be called the upper or top channel, and the second channel may be called the lower or bottom channel. Other materials may be required for special needs.

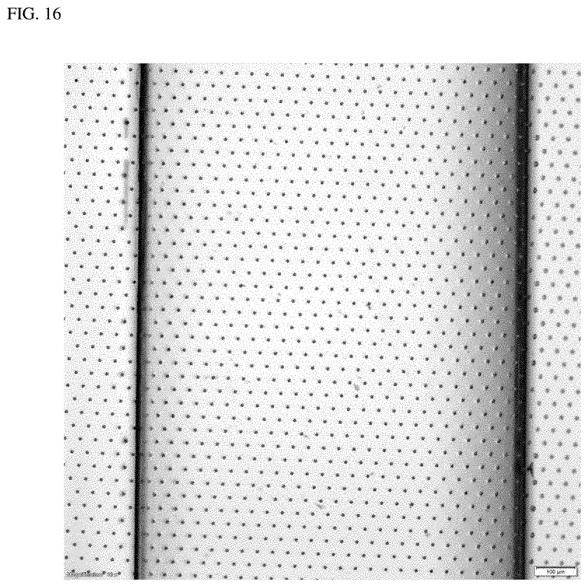

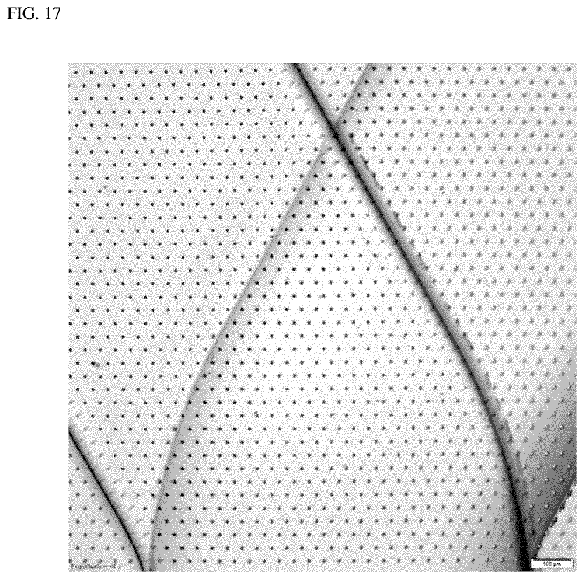

The protocol:
1. Place a perfusion manifold assembly with microfluidic device attached, or Emulate Pod™ with an Emulate Organ Chip attached, under a microscope condenser. Start by using the 10× objective.
2. Focus the microscope on the membrane, distinguished by hexagonally-packed pores, as seen in FIG. 16. Move the perfusion manifold assembly, or Emulate Pod™, until the objective is clearly under the microfluidic device, or Emulate Organ Chip, channel area.
3. Adjust fine-focus, moving slightly upwards to locate cells of interest in the top channel of the microfluidic device, or Emulate Organ Chip. It may be easier to identify cells in the top channel when looking at the co-culture region. In other words, it may be simpler to identify the cells in the top channel when looking at the region where the top channel and bottom channel do overlap, with the membrane between them. In order to visualize the cells in the bottom channel, the clearest region is usually in the inlet or outlet of that bottom channel, as seen in FIG. 17. In other words, it may be simpler to identify the cells in the bottom channel when looking at the portions of the bottom channel where the top channel and bottom channel do not overlap.

FIG. 16 is an exemplary microscopic image of the co-culture region of a microfluidic device. The membrane may be identified by the presence of membrane pores.

FIG. 17 is an exemplary microscopic image of the region where one channel may begin or end to overlap a second channel in a microfluidic device. The membrane may be identified by the presence of membrane pores.
4. Once the appropriate region and cell type has been identified, adjust the magnification as desired, then re-focus the fine-focus adjustment.
5. Inspect the entire length of the channel at the desired magnification to assess morphology of the cells and uniformly of cell quality throughout the microfluidic device, or Emulate Organ Chip. The entire length of the channel should be inspected to produce good data.
6. For cells in the top channel, acquire images at three different areas of the channel, such as inlet, middle and outlet positions. The middle region can be localized by the presence of working or vacuum channels if they are present in the microfluidic device. If the working or vacuum channels are present, they may be located perpendicular to the main channel of the microfluidic device.
7. For cells in the bottom channel, inspect cell morphology and acquire images at three different areas of the channel, such as inlet, middle and outlet portions. Imaging the cells in the bottom channel may be impeded by the cells in the top channel.
8. Assess the morphology at any time during the culture process and track any changes in cellular morphology over time.

The above protocol may be adapted for use with a high-content imaging workflow.

### Exemplary Vimentin Staining Protocol

Vimentin may be used to mark quiescent stellate cells that are not activated, in principle the opposite of α-SMA. Vimentin and α-SMA staining is useful to image all stellate cells so that the proportion of activated cells may be gauged.

Immunofluorescent staining of microfluidic devices is used for a variety of experiments. Herein, the goal of the immunofluorescent staining is to visualize vimentin via fluorescent imaging in fixed microfluidic devices, such as an Emulate Organ Chip. Required materials include anti-vimentin antibody [SP20] (Abeam ab16700), 10% Saponin, phosphate buffer solution (PBS), bovine serum albumin (BSA), normal goat serum (or other serum from the species the secondary antibody was raised in), Alexa Fluor®488-conjugated goat anti-rabbit IgG secondary antibody (or other anti-rabbit secondary), and a fluorescent microscope. Fixed microfluidic devices are also needed for the imaging. Fixed samples are adherent to the surface on which they are to be imaged. The recommended fixative is 4% paraformaldehyde (PFA) for 15 minutes at room temperature. The recommended permeabilization is 1% Saponin in PBS for 30 minutes at room temperature. The recommended blocking buffer is 1% BSA and 10% goat serum in PBS incubated overnight at 4° C. The recommended antibody host is rabbit. The recommended secondary antibody dilution is Alexa Fluor0488-conjugated goat anti-rabbit IgG secondary antibody 1:500 dilution in blocking buffer for 2 hours at room temperature in the dark.

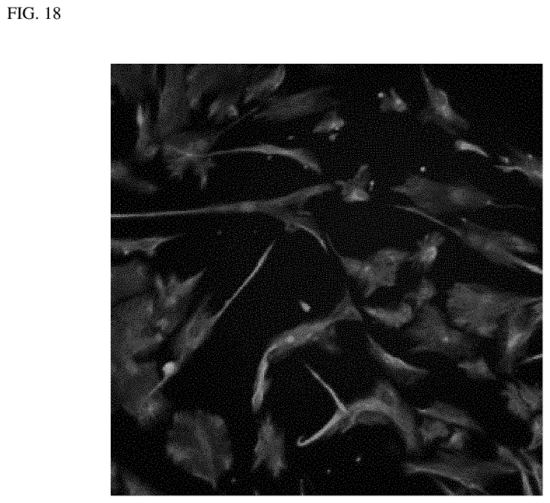

FIG. 18 is a representative image of human stellate cells stained by vimentin (green) in the bottom channel of a microfluidic device, specifically an Emulate human Liver Chip.

The above protocol, and other protocols described herein, may be used with the high-content imaging protocol presented herein, as well as during regular microscopy.

### Exemplary Stabilin-1 Staining Protocol

Stabilin-1 is a mark of the liver sinusoidal endothelial cells, specific to fenestrated endothelial cells. Staining for Stabilin-1 may be useful in imaging so as to ease detecting and counting of liver endothelial cells. This gene encodes a large, transmembrane receptor protein and is primarily expressed on sinusoidal endothelial cells of liver, spleen, and lymph node. The receptor has been shown to endocytose ligands such as low density lipoprotein, Gram-positive and Gram-negative bacteria, and advanced glycosylation end products.

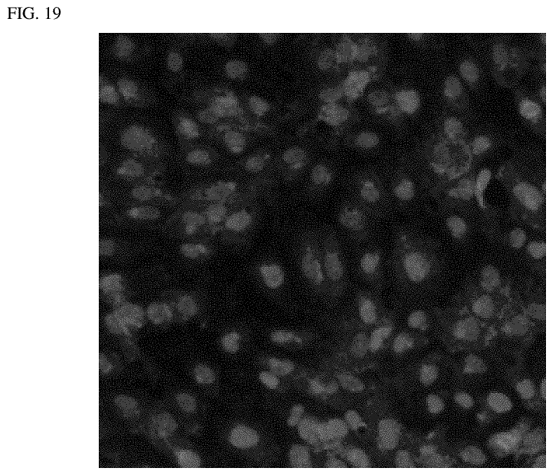

Immunofluorescent staining of microfluidic devices is used for a variety of experiments. Herein, the goal of the experiment is to visualize the multifunction al scavenger receptor stabilin-1 in microfluidic devices, such as the Emulate Organ Chip. Required materials include Anti-Stabilin-1 (STAB1) antibody (Novus NBP1-84444), 4% paraformaldehyde (PFA), 10% saponin, phosphate buffer solution (PBS), bovine serum albumin (BSA), normal goat serum (or other serum from the species the secondary antibody was raised in), and Alexa Fluor®555-conjugated goat anti-rabbit IgG secondary antibody (or other anti-rabbit secondary antibody.) The recommended fixative is 4% paraformaldehyde (PFA) for 15 minutes at room temperature. The recommended permeabilization is 1% Saponin in PBS for 30 minutes at room temperature. The recommended blocking buffer is 1% BSA and 10% goat serum in PBS incubated overnight at 4° C. The recommended antibody host is rabbit. The recommended primary antibody dilution is Anti-Stabilin-1 (STAB!) (Novus NBP1-84444) at a 1:50 dilution in blocking buffer incubate overnight at 4° C. The recommended secondary antibody dilution is Alexa Fluoro555-conguated goat anti-rabbit IgG secondary antibody at a 1:500 dilution in blocking buffer incubated at room temperature for 2 hours in the dark. FIG. 19 is a representative image of a STAB1 (red) and nuclei staining (blue) indicating the presence of LSEC cells in the bottom channel of a microfluidic device, specifically an Emulate human Liver Chip.

FIG. 19 is a representative image of a STAB1 (red) and nuclei staining (blue) indicating the presence of LSEC cells in the bottom channel of a microfluidic device, specifically an Emulate human Liver Chip.

The above protocol, and other protocols described herein, may be used with the high-content imaging protocol presented herein, as well as during regular microscopy.

### Target Gene Expression and Delivery of Encapsulated or Decorated Molecules in the Liver-Chip

In some embodiments, liver-chips, or microfluidic devices seeded with liver cells, are used for testing intracellular delivery of therapeutic compounds. Thus, in some embodiments, liver-chips were transduced with lipid nanoparticles (LNPs). Transduction refers to a process by which heterologous (e.g. not naturally found in the host cell) DNA or RNA, e.g. mRNA, is introduced into a host cell by a virus or viral vector. Lipid nanoparticles used herein may be encapsulated and/or decorated, i.e. LNPS may have cell type specific attachment molecules for binding to cell surface receptors of target host cells.

Lipid nanoparticles include but are not limited to recombinant encapsulated viral vectors, e.g. Adeno-Associated Virus (AAV), etc. Moreover, recombinant viral vectors, are not limited to one serotype, indeed, numerous serotypes may be used, e.g. for AAV serotypes include but are not limited to AAV2, AAV8, AAV9, etc.

In some embodiments, encapsulated LNPs comprise a recombinant viral vector and at least one type of recombinant RNA sequence. In some embodiments, LNPs comprise a recombinant viral vector and at least two types of recombinant ribonucleic acid (RNA) sequences. In some embodiments, recombinant RNA sequences include but are not limited to messenger RNA (mRNA), regulating RNA, including but not limited to silencing RNA, e.g. small interfering RNA (siRNA), RNA interference (RNAi), etc. In some embodiments, recombinant RNA sequences are intended as a therapeutic, e.g. RNAi for reducing ASGR1 expression, e.g. as an anti-viral therapy. In some embodiments, recombinant RNA sequences are intended for co-delivery of a therapeutic. In some embodiments, recombinant mRNA sequences are intended for expressing a reporter molecule, e.g. mRNA expressing green fluorescent protein (GFP), i.e. GFP transgene.

In some embodiments, liver-chips may be seeded with hepatocytes, including but not limited to human, rat, monkey, e.g. Cynomolgus monkey—Macaca fascicularis, etc. In some embodiments, monkey hepatocytes are derived from the monkey liver tissue. In some embodiments, monkey liver tissue is obtained from biopsy material. In some embodiments, monkey hepatocytes are obtained from a commercial sources, e.g. Cell Biologics. In some embodiments, monkey hepatocytes are cultured in Hepatocyte Medium/w Kit (500 ml), e.g. Cell Biologics. In some embodiments, monkey hepatocytes seeded into microfluidic liver chips are primary cells. In some embodiments, monkey hepatocytes seeded into microfluidic liver chips are derived from primary cells. In some embodiments, liver-chips may be seeded with or without endothelial cells, as described herein. In some embodiments, liver-chips may be seeded with or without Kupffer cells and with or without hepatic stellate cells. In some embodiments, liver-chips may be cultured under physiological fluid flow.

In some embodiments, liver-chips as described herein, may be transduced with LNPs for evaluation of dose and time-dependent transduction determined by expression of a recombinant reporter molecule, e.g. GFP. In some embodiments, liver-chips transduced with LNPs are used for safety assessment of AAV vectors. In some embodiments, liver-chips transduced with LNPs are used for personalized medicine.

In many in vitro platforms, human hepatocytes display rapid downregulation of gene expression. Therefore in vivo models are mainly used for identifying downregulated genes and proteins, e.g. rats. Thus, there remains a need for an in vitro human platform, i.e. hepatocyte model, for demonstrating a treatment associated gene downregulation that is not a gene downregulation effect merely by culturing hepatocytes over the assay time period.

One exemplary biomarker expressed by liver cells, i.e. hepatocytes, is a cell surface Asialoglycoprotein receptor 1 (ASGR1), a hetero-oligomeric protein composed of major and minor subunits. Asialoglycoprotein receptor 1 refers to a transmembrane protein that plays a role in serum glycoprotein homeostasis by mediating one or more of the binding of, internalization, endocytosis, then lysosomal degradation of certain glycoproteins. Such glycoproteins (ASGR1-ligands) have one or more exemplary units which may be used for decorating LNPs, e.g. terminal galactose (Gal), β-linked galactose, N-acetylgalactosamine residues, oligosaccharide chains, etc. Asialoglycoprotein receptors may facilitate hepatic infection by multiple viruses including hepatitis B, hepatitis C, etc. Thus in one embodiment, ASGR1 is known to mediate hepatic binding and uptake of natural hepatitis B virus (HBV). Further, ASGR1 is used as a target for liver-specific drug delivery. Thus in one embodiment, ASGR1 plays a role in virus and RNA uptake (RNA therapeutic applications). In one embodiment, a therapeutic RNA delivered to a hepatocyte lowers ASGR1 expression.

For testing of a treatment associated downregulation of a biomarker expressed by hepatocytes, ASGR1 was chosen as an exemplary biomarker. Human hepatocytes were cultured for 10 days in the liver-chip, then stained (including immunostaining and 4′,6-diamidino-2-phenylindole (DAPI)) then imaged for presence and location within or on liver cells. After investigation, it was found that it was present and localized to hepatocytes.

Thus, in on embodiment, a liver-chip platform may be used for monitoring ASGR1 expression was contemplated for use in evaluating a RNAi therapeutic against ASGR1 protein.

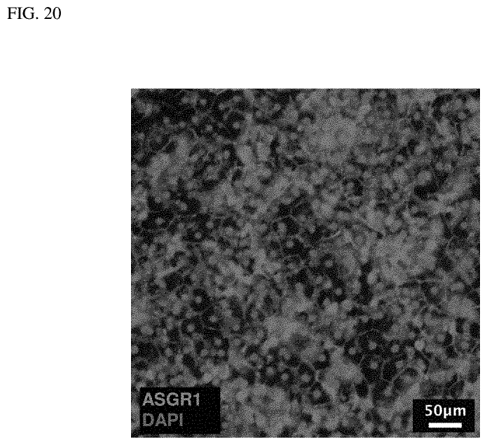

FIG. 20 shows exemplary ASGR1 found to be present and localized within hepatocytes after 10 days in culture in the Liver-Chip. Human Liver-Chip—Day 10 in Culture. Pink—ASGR1 protein. Nuclei colored blue (DAPI).

The above protocol, and other protocols described herein, may be used with the high-content imaging protocol presented herein, as well as with regular microscopy.

Formulated mRNA Delivery in the Liver-Chip

In one embodiment, an investigation was done for addressing whether human hepatocytes in the Liver-Chip can be transfected with LNP-encapsulated mRNA expressing a GFP fluorecent marker. LNPs containing green fluorescent protein (GFP) mRNA were introduced into a liver chip into the apical (hepatocyte) channel. The expression profile of GFP was in line with in vivo rodent studies: LNP #1 induced GFP expression that started high, e.g. up to 8-24 hours post exposure, and gradually tapered off over 48, 72 and 96 hours, while LNP #2 caused delayed expression over 8-24 hours that peaked after 48 hours, then diminished over 72-96 hours. Thus, GFP mRNA delivered to hepatocytes in the apical channel resulted in LNP-specific GFP expression patterns.

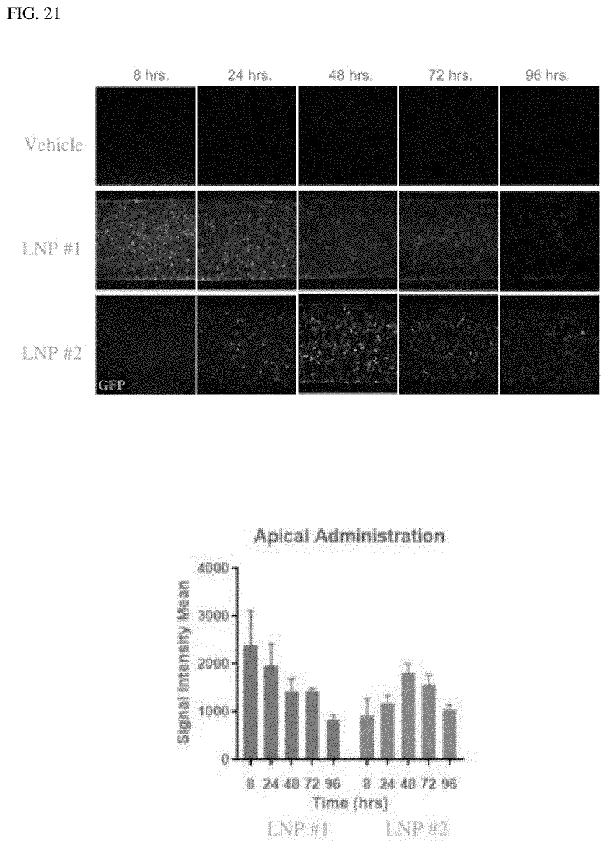

FIG. 21 shows exemplary results after LNPS comprising GFP mRNA were delivered to human hepatocytes in the apical channel. Fluorescent images are shown over time from the apical channel where two types of LNPs comprising GFP mRNA were delivered in the apical channel resulting in LNP-specific GFP expression patterns. No signal was observed in vehicle treated cells or in the cells located in the basal channel under apical administration. Green shows exemplary GFP expression. The lower chart shows exemplary GFP signal over time for LNP #1 and LNP #2.

The above protocol, and other protocols described herein, may be used with the high-content imaging protocol presented herein, as well as with regular microscopy.

### Adeno-Associated Virus (AAV) Delivery of GFP Transgene

In vitro hepatocytes cultured in other systems are highly resistant to AAVs, making it difficult to study of the effect of AAV vectors in human and cynomolgus (cyno) monkey hepatocytes. As a proof of concept, we conducted a pilot study where we investigated whether hepatocytes in human and cyno Liver-Chips can be transduced with AAVs containing a GFP transgene, to determine the potential application of the Liver-Chip for safety assessment of AAV vectors.

Three (3) serotypes of AAV were evaluated, and 3 of 3 displayed dose and time-dependent transduction in both human and cyno Liver-Chips. Dose dependent data is shown in

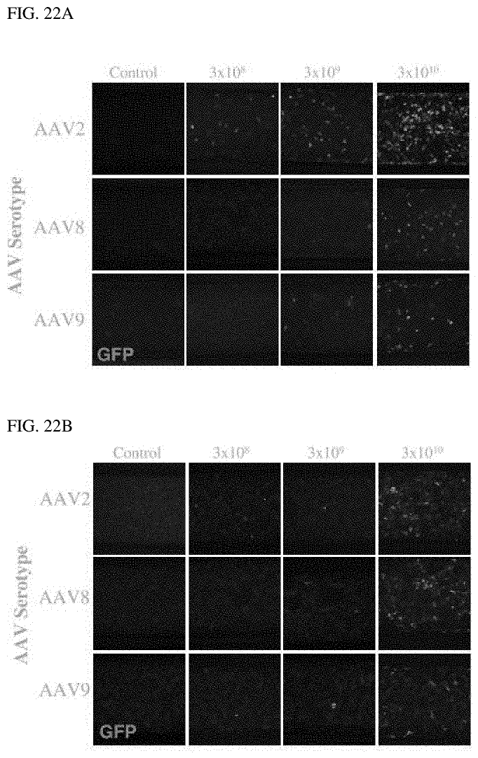

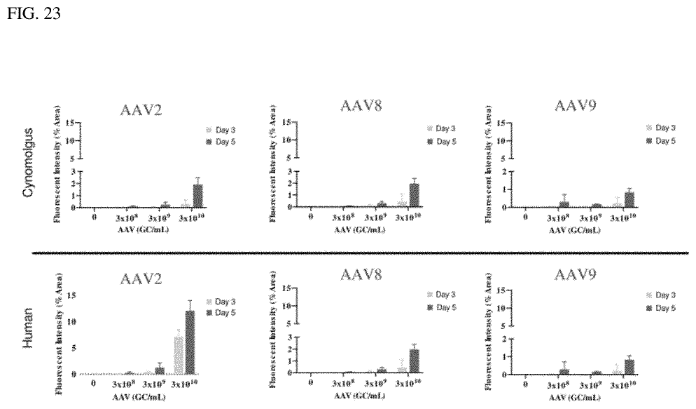

FIG. 22A-22B. Time-dependent data shown in FIG. 23. Thus, AAV LNPs can transduce microfluidic liver chips, i.e. deliver genetic sequences into hepatocytes cultures on-chip. In some preferred embodiments, hepatocytes cultured in liver chips are transduced with LNPs.

FIG. 22A shows immunofluorescent images of GFP expression in hepatocytes as dose dependent in Human Liver-Chips, tested as controls (no LNPs), 3×10⁸, 3×10⁹, and 3×10¹⁰ as AAV concentration of genome copies per mL-GC/mL.

FIG. 22B shows immunofluorescent images of GFP expression in hepatocytes as dose dependent in Cynomolgus (monkey) Liver-Chips. AAV concentration (GC/mL).

FIG. 23 demonstrates that 3 AAVs vectors, AA2, AAV8, AAV9, displayed time dependent transduction in cynomolgus and human Liver-Chips. Day 3 vs. Day 5, for 3×10⁸, 3×10⁹, and 3×10¹⁰ AAV genome copies per mL-GC/mL.

The above protocol, and other protocols described herein, may be used with the high-content imaging protocol presented herein, as well as with regular microscopy.

### Exemplary Experimental Method

### I. Materials and Methods

9. Preparation of Microfluidic Devices Seeded with Liver Cells

Microfluidic devices were obtained from Emulate Inc. (USA) and are illustrated in FIG. 1A. The channels of the microfluidic devices were coated with a proprietary mixture of extracellular matrix and were incubated overnight at 37° C. Primary human cryopreserved hepatocytes sourced from ThermoFisher, as one example, were seeded in the top channel at a density of 3.5×10⁶ cells/mL, and were left to form an attached monolayer at 37° C. in a humidified incubator with a 95%/5% ratio of air to CO₂.

In some embodiments, cryopreserved hepatocytes are thawed into Complete Hepatocyte Seeding Medium, then centrifuged through a 90% Percoll (colloidal silica particles of 15-30 nm diameter (23% w/w in water) which have been coated with polyvinylpyrrolidone (PVP), Sigma) solution in 10× DPBS (−/−). Cells are rinsed several times in Complete Hepatocyte Seeding Medium then used to seed plates or microfluidic chips.

Twenty-four hours after seeding, the hepatocyte monolayer was overlaid with Matrigel Matrix sourced from Corning, USA. Primary human cryopreserved liver sinusoidal endothelial cells, also known as LSECs, were seeded on the bottom channel of the microfluidic device at a density of 2 to 4×10⁶ cells/mL in endothelial medium, also known as CSC medium or Base LSEC Culture Medium; CSC basal Medium plus Culture-Boost (2%), Cell Systems, and Pen/Strep (1%), Sigma. In some embodiments, endothelial cells are cultured in Complete LSEC Culture Medium, Base LSEC Culture Medium plus 10% FBS.

Microfluidic devices were inverted for 2 hours to allow attachment underneath the membrane, and then inverted back before being connected to a platform containing a pneumatic pump source from Emulate Inc. Media was flowed through both channels at a flow rate of 30 μL/hour and was refreshed every other day.

The effects of agents, such as staurosporine on cell phenotype was evaluated. Microfluidic devices were perfused with staurosporine (0 and 10 μM) for 6 hours at a flow rate of 30 μL/hour. A compound was also tested in the microfluidic devices seeded with liver cells. The microfluidic devices seeded with liver cells were perfused with the compound at the concentrations of 0.1 μM, 1 μM or 10 μM for 3 hours, after which the compound was removed from the media flow. The protocol was chosen as it was analogous to how the compound has been evaluated in vivo. After a further 3 or 21 hours with compound-free media flow, microfluidic devices were fixed with formaldehyde and stored in phosphate buffered saline (PBS) at 4° C. as described below.

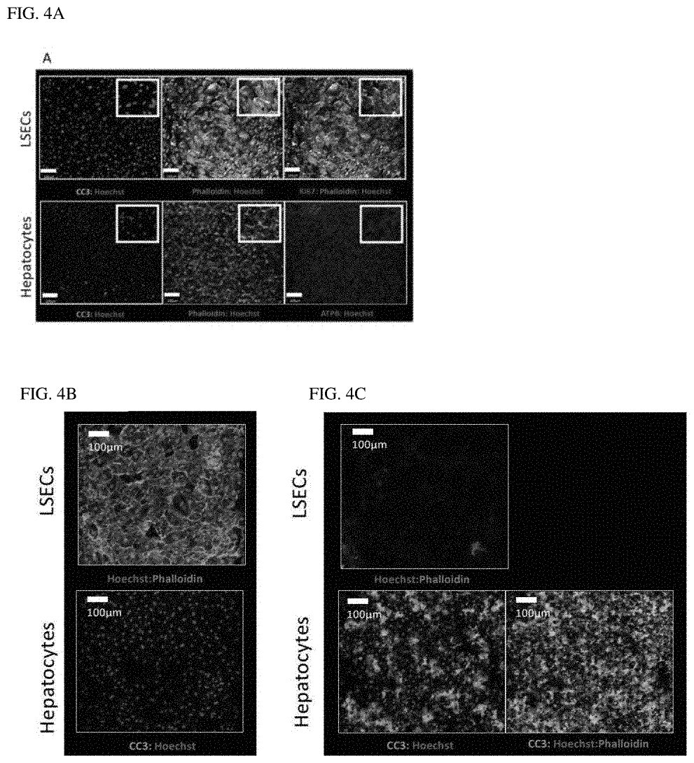

FIGS. 4A-4C show exemplary confocal images taken at 20× magnification. Images are representative and taken from the mid region of each microfluidic device. Images shown include markers of F-actin morphology (Phalloidin), proliferation (Ki67), mitochondrial structure (ATPB—ATP synthase beta subunit), apoptosis (CC3—Cleaved Caspase 3) and Nuclei (Hoechst). FIG. 4A shows images captured from a microfluidic device treated with vehicle control (DMSO). FIGS. 4B-4C show images are taken from the mid region of microfluidic devices and show markers of F-actin morphology (Phalloidin), apoptosis (CC3) and Nuclei (Hoechst). Images were captured from microfluidic devices treated with vehicle control (DMSO) or staurosporine (10 μM) for 6 hours. FIG. 4B shows the microfluidic devices treated with vehicle control (DMSO). FIG. 4C shows microfluidic devices treated with staurosporine (10 μM).

10. Preparation of Microfluidic Devices Seeded with Kidney Cells

Dual-channel microfluidic devices were obtained from Nortis Inc, USA, and were prepared as previously described [10]. Briefly, the microfluidic devices were filled with a collagen I matrix and subsequently coated with human collagen IV, after overnight incubation at room temperature. Primary human cryopreserved proximal tubule epithelial cells, sourced from Biopredic, were cultured according to supplier specifications and injected in the left channel of the microfluidic devices as a single cell suspension at a density of 1.0×10⁶ cells/mL, or 5 μL per microfluidic device. Microfluidic devices were kept in a humidified incubator with 95%/5% ratio of air to CO2 at 37° C. for 24 hours to allow the cells to adhere to the collagen matrix. Media reservoirs were filled with 10 mL cell culture media and flow was initiated in the Nortis perfusion platform, sourced from Nortis Inc., at a rate of 0.5 μL/min. Microfluidic devices were cultured for 7 days and media reservoir waste levels were monitored daily to ensure adequate perfusion. Tube formation and morphology were monitored daily by visual inspection using a bench transmitted-light microscope.

### 11. Immunofluorescence

The microfluidic devices seeded with liver cells and microfluidic devices seeded with kidney cells were fixed with 4% formaldehyde for 15 and 30 minutes respectively, at room temperature before being washed and permeabilized with Triton X 0.1% with 2% BSA in phosphate buffered saline. Blocking and incubation with the primary antibody, overnight in PBS containing 2% bovine serum albumin, was followed by 2 hours of incubation with fluorescently conjugated secondary antibodies, sourced from Invitrogen, also containing Phalloidin-488 with a ratio of 1:500, sourced from Thermofisher, and Hoechst 33342 with a ratio of 1:1000, sourced from Invitrogen, in the same buffer. Immunostaining of the microfluidic devices seeded with liver cells was performed with primary antibodies specific for the beta subunit of the ATP synthase enzyme (ATPB; 1:100, Abcam, ab14730), Ki67 D3B5 (1:50, Cell Signalling 12075S) and Cleaved Caspase 3 (CC3) Asp175 (1:100, Cell Signalling, 9661S). An antibody specific for Zonula-occludens 1 (ZO1) (1:100, BD Biosciences) along with Phalloidin-488 (1:500) was used in the microfluidic devices seeded with kidney cells.

### 12. Design and Printing of Adaptors

Microfluidic device adaptors, otherwise known as microfluidic device holders, were designed and 3D printed to allow compatibility with the plate based, automated confocal microscope and may be seen in FIG. 2. Two microfluidic device holder designs were created to capture the different formats of microfluidic device. FIG. 2 shows the production parts for the microfluidic devices seeded with liver cells. Designs were made in house using SolidWorks 3D CAD and printed on a Stratasys OBJET30 Prime 3D printer. Parts were made using a Stratasys OBJET MED610 Biocompatible material (Part # OBJ-04057).

### 13. Confocal Image Acquisition

Microfluidic devices were imaged on a Cell Voyager 7000 (CV7000, Yokogawa Inc.). Confocal fluorescent images were captured using a long working distance 20× objective (Olympus LUCPLFLN 0.45 NA, WD 6.6-7.8 mm) and an Andor Neo sCMOS camera with a 2×2 bin. Hoechst was imaged using a 405nm excitation laser (405±5 nm, 100 mW, Coherent) with a 445/45 nm band pass emission filter. CC3 and ZO1 were imaged using a 561nm excitation laser (561±2 nm, 200 mW, Coherent) with a 600/37 nm band pass emission filter. Phalloidin was imaged using a 488 nm excitation laser (488±2 nm, 200 mW, Coherent) with a 525/50 nm band pass emission filter and Ki67/ATPB imaged using a 640 nm excitation laser (640 4-5 nm, 100 mW, Coherent) with a 676/37 nm band pass emission filter.

For the microfluidic devices seeded with liver cells, images were captured over a 120 μm range at 5 μm Z intervals to cover both the LSECs and hepatocyte cell layers. For the microfluidic devices seeded with kidney cells, images were captured over a 120 μm range at 1 gm Z intervals. In the first pass scan, bright-field images were acquired using a 4× objective (Olympus UPLSAPO 0.16 NA, WD 13 mm) using a 100 W Halogen lamp as an illumination source.

### 14. Image Analysis

a. Intelligent and High-Content Scanning Algorithm

For control of the CV7000 automated confocal microscope, the SearchFirst functionality from the Wako Software Suite, sourced from Wako Automation, was used, which defines a workflow where a first round of acquisition takes place, followed by launch of an analysis script, which calculates coordinates from the first-round images. The coordinates are then passed to the microscope for a second round of acquisition at higher magnification. A simplified process is as follows:
1. A first low-resolution round of acquisition
2. Calculation of coordinates bases on the first round of acquisition
3. A second high-resolution round of acquisition based on the calculated coordinate system

In the first pass, seven bright field images were captured for each microfluidic device seeded with liver cells. To define the same field of view, abbreviated as FOV, on every microfluidic device, a common coordinate system was defined, based on features which are present in each microfluidic device seeded with liver cells, as seen in FIG. 3A-B. The images were corrected for variation in illumination across each image, then stitched together to form one image covering the whole of the microfluidic device area. A Hessian-based trough detection was then applied to enhance dark lines in the image, followed by a Radon transform to detect straight lines in the images, as seen in FIG. 3A. The edges of the main channel were detected as two lines close to the vertical orientation and with the expected separation, defining the horizontal location of the main channel. The working channels to either side of the main channel were detected as two lines perpendicular to the main channel, with the correct spacing. Together, these lines defined the standard coordinate system for the microfluidic device seeded with liver cells. Coordinates equally spaced along the center of the main channel were chosen, with a gap marginally larger than the size of the FOV. Points defined in these coordinates will lie at the same position on every microfluidic device.

The general approach of identifying landmarks at low magnification can be applied to any microfluidic device architecture. Intelligent, high-content scanning, otherwise known as high-content, of microfluidic devices to be seeded with kidney cells from a different manufacturers was conducted in order to illustrate that the method may be applied across different microfluidic device architectures. To generalize the approach for any microfluidic device system, one needs to apply the following three steps. First, a round of low-resolution images are taken in order to create a reference image set. Second, coordinates of fields of interest are placed manually on a reference image set. Third, for the intelligent scanning run, the first pass images were aligned to the reference image using a rigid registration algorithm, to determine where to place the field coordinates on the first pass images. To account for brightness and focus variations in the bright field images, both reference and test images were normalized, smoothed and edge filtered, before performing the registration.
b. Quantification of Fluorescent Images

In the microfluidic device seeded with liver cells, the fluorescent images were acquired as a single stack comprising the endothelial layer and hepatocyte layer together, separated by the membrane. First, the stack was separated into two layers by finding the minimum in the Hoechst channel along the z-direction. To account for variations in layer locations across the image the image was broken down into 4×4 sub regions, the height of the minimum was located in each region and then this height was interpolated over the whole image, giving a surface, which separated the two layers as shown in FIG. 3B. For morphological measurements of hepatocytes and nuclei, images were segmented in three dimensions, and measures of cell number and organization were computed. For fluorescence measurements, a maximum projection was calculated for each layer, and cell regions were segmented using the phalloidin channel, starting from nuclear regions as seeds, to calculated single cell intensity values. Where appropriate, the fluorescence images were separated into distinct length scales of signal using morphological opening operations.

### 15. Statistical Analysis

A framework of statistical best practice for microfluidic device studies to reduce bias and variability has been developed. Principles of optimal experimental design to randomize the order in which the microfluidic devices are imaged was used. The principles of optimal experimental design distributed experimental conditions equally across the different rows within a microfluidic device holder and between individual holders to reduce bias associated with microfluidic device location or imaging order.

The relationship between the image analysis endpoints and treatment, or time effects while controlling for the microfluidic device to microfluidic device variability, were analyzed, as well as row or holder effects. Specifically, a Bayesian multilevel linear regression model with uninformative priors was fitted, treating the fluorescence signal of the fields of view of a microfluidic device as repeated measurements. It was estimated that treatment and time effects as well as the variability induced by microfluidic device, holder row and each individual holder itself. Bayes p-values were reported, each representing the probability that the true effect is in the opposite direction than what was considered. Statistical significance was accepted with a Bayes p-value of less than 0.025. The analysis was implemented using the “brms” package for the R programming language.

In some embodiments, modeling data from the microfluidic devices with a positive control for inducing a cytotoxicity in hepatocytes is contemplated for use in demonstrating a power analysis enabling identification of the minimum sample size necessary to detect a given phenotypic effect. To do so, data from the fitted model, a parametric bootstrap, was simulated where the treatment effect relative to the positive control and the sample size was varied. Summary statistics on the simulations to assess the power of the statistical analysis was used.

### II. Results

An end-to-end workflow that enables the automation of confocal microscopy of microfluidic devices to reduce user input and experimental bias, and increase throughput has been developed. Furthermore, statistical modelling to create a framework that guides future experimental design was applied. The improvements in throughput and reproducibility move towards the possibility of using microfluidic devices in a drug screening setting.

i. Imaging Workflow of Microfluidic Device Seeded with Liver Cells Model

An outline of the workflow is illustrated in FIG. 3A-B. Low magnification, bright field images were acquired and tiled to cover the entire microfluidic device. After the first pass, a script was automatically launched in MATLAB, which tiled the images and identified the location of 30 standard FOV corresponding to the same locations on each microfluidic device. These coordinates were used in the second pass of imaging, at higher magnification, multiple fluorescent channels and a three-dimensional Z-stack to capture both cell layers of the microfluidic devices seeded with liver cells. The whole process is fully automated, requiring no user intervention other than to load the microfluidic devices into the adapters. Each holder can contain up to eight microfluidic devices and the imaging acquisition process takes 70 minutes per holder, but requiring less than five minutes of user interaction time. As well as increasing the number of microfluidic devices that can be imaged within a single study, this approach allowed more FOV to be acquired, increasing the amount of data collected from a single microfluidic device. The FOV were defined to be in the same location on every microfluidic device, enabling standardization of acquisition and statistical modelling of the sources of uncertainty in microfluidic devices measurements, such as cell seeding and microfluidic device inconsistencies, and potential spatial effects such as zonation, which are not systematically studied in microfluidic device seeded with liver cells systems unless oxygen biosensors are incorporated.

ii. Capturing Cellular Phenotype Changes Following Drug Exposure

Exemplar confocal fluorescent images from the higher resolution, second pass imaging step of both the hepatocyte and endothelial cell layers on the microfluidic device seeded with liver cells are shown in FIG. 4A-4C. It is possible to obtain a clear separation of the cell layers with single cell resolution as well as signals for well validated stains of cellular morphology, via F-actin visualization by Phalloidin staining, proliferation (Ki67), apoptosis (CC3) and mitochondrial structure (ATP synthase beta subunit, ATPB).

Bosentan is a potent inhibitor of Bile salt efflux pump (BSEP) and causes downregulation of BSEP expression as well as Multidrug Resistance-associated Protein (MRP2) expression. Thus, imaging using fluorescent bile acids is contemplated.

Imaging techniques were performed on microfluidic devices seeded with liver cells treated with staurosporine, a compound reported to induce apoptosis in hepatocytes through activation of caspsase-3 [9]. Staurosporine (10 μM) produced marked increases in the apoptotic marker (CC3) with little if any effect on hepatocyte cell number and complete loss of LSECs after six hours of exposure (FIG. 4B).

iii. Statistical Analysis: Proof of Concept and Experimental Design

In some embodiments, to inform future experimental design, a power analysis was contemplated to compute the number of replicate microfluidic devices required to ensure sufficient statistical power to detect a hepatotoxic effect. It is common practice to aim for at least 80% power, therefore this analysis contemplates, in one embodiment, that for endpoints of interest at least three replicate microfluidic devices are required per condition to achieve sufficient statistical power to detect a phenotype comparable to 80% of the positive control.

iv. Decoupling Sources of Variability

To demonstrate how to apply this workflow for the safety profiling of potential drugs, the hepatotoxic effect of an active drug candidate currently in clinical development was analyzed.

Eighty microfluidic devices that contained hepatocytes from two different human donors were analyzed at 3-time points (3 hours, 6 hours and 24 hours) and four concentrations (vehicle, 0.1 μM, 1 μM, 10 μM), with each group consisting of at least three replicate microfluidic devices. As described above, a Bayesian multilevel linear regression model was fit. The high number of microfluidic devices imaged allowed different sources of variability to be decoupled, including microfluidic device, holder row and individual holder effects, by including hyperparameters describing the standard deviation associated with each error source. Accordingly, the fitting procedure automatically identified the microfluidic device to microfluidic device, row-to-row and holder-to-holder variability, and allowed biological treatment effect after adjusting for these unwanted influences to be estimated. FIG. 5A-5B displays the posterior probability densities estimating the standard deviation corresponding to the different sources of variability as well as the treatment and field effects for two representative endpoints: LSEC cell count and hepatocyte CC3 positive fraction. FIG. 5A illustrates that the highest source of variability with respect to LSEC cell count comes from microfluidic device to microfluidic device variability, as the posterior density was high and comparably tight (in contrast, the holder-to-holder and row-to-row variability's posterior densities were wide spread and close to zero, indicating low holder-to-holder and row-to-row variability). This is an encouraging result, indicating that the holder and imaging system are not a significant source of noise in the system for this sort of measurement. In contrast, FIG. 5B illustrates that the highest source of variability with respect to hepatocyte CC3 response was the variability across holders, i.e. the order in which the microfluidic devices are imaged. This highlights the randomization of the position in which the microfluidic devices are placed in the holders and hence the order in which they are imaged.

With the various sources of measurement error characterized, an adjusted estimate of the treatment effect at each concentration and timepoint was derived. It may be concluded that the drug treatment showed no significant effect at any of the endpoints that were analyzed. Additionally, these holders prove advantageous to standard microscope stage adapters, as they reliably place microfluidic devices in consistent positions, such that variability is significantly lowered experiment to experiment.

v. Confirmation of Approach Using Microfluidic Devices from a Different Manufacturer

Traditional in vitro renal cell cultures lack functionality and retain a poor epithelial phenotype [15]. Microfluidic devices have been designed to provide renal proximal tubule cells with a microenvironment that has continuous luminal flow promoting the self-assembly of a tight epithelium and recreating the characteristic barrier function [16]. Imaging and reconstruction of a whole kidney tubule is a powerful tool to understand how nephrotoxicity develops over time, where the injury is manifest and impact on individual cells. The potential to multiplex high resolution imaging with quantitative analysis of metabolites and injury markers in microfluidic devices seeded with kidney cells can dramatically improve the reach of these systems in renal research and disease mechanism elucidation.

Using 3D printing bespoke adaptors were created to enable the automated confocal imaging of these microfluidic devices seeded with kidney cells (see FIG. 6A-6D). To improve the flexibility of the intelligent scanning algorithm, avoiding the need to design custom image processing for different microfluidic device designs, placed FOV coordinates were manually placed on a single reference image. First-pass scans are then registered to this reference image, aligning based on strong edges in the images, to determine where to place the fields for the second pass. By basing the algorithm on global features of the image, this approach improves the robustness of the field identification, because some elements can be missing or altered and the alignment will still be successful. FIG. 6B shows the location of the fields identified for high resolution imaging in the second pass, overlaid on a stitched image of the four first pass bright field images. Slices from the high resolution second pass stacks are also displayed in FIG. 6D along with a three-dimensional reconstruction of the proximal tubule (FIG. 6C).

FIGS. 6A-6D show automated confocal imaging of microfluidic devices seeded with kidney cells. FIG. 6A shows a schematic of microfluidic device seeded with kidney cells. FIG. 6B shows a brightfield image (4× magnification) from first pass acquisition with defined fields of view highlighted (green) for second pass imaging. FIG. 6C shows a 3D render of proximal tube (rendered from 20× magnification Z stack images). FIG. 6D shows exemplar confocal images taken from the mid region of a kidney tubule taken at 20× magnification. Cells have been stained with Phalloidin (green), ZO1 (orange) and Hoechst (Blue).

### Study Conducted Using High-Content Imaging Workflow

### I. Introduction

Preclinical rodent and dog toxicity models required by regulatory agencies often do not produce consistent results or predict complications in humans, leading to high rates of drug failure in the clinic. Here dual-channel, microfluidic devices seeded with cells were applied to construct rat, dog, and human microfluidic devices seeded with liver cells containing species-specific primary hepatocytes interfaced with liver sinusoidal endothelial cells, with or without Kupffer cells and hepatic stellate cells, cultured under physiological fluid flow. The microfluidic devices seeded with liver cells detected species-specific toxicities, including necrosis, fibrosis, cholestasis, Kupffer cell depletion, and innate immune response when treated with multiple drugs. Multi-species microfluidic devices seeded with liver cells may therefore provide a useful platform to inform decisions on therapeutic compound progression by better defining human relevance of liver toxicities detected in animal studies.

The U.S. Food and Drug Administration (FDA) and European Medicines Agency generally require that the safety of new drug candidates be evaluated in a rodent and a non-rodent model, frequently rat and dog, before moving the compound into clinical trials. However, analysis of 150 drugs that caused adverse events in humans found that regulatory testing in rats and dogs only correctly predicted 71% of toxicities in humans. Moreover, while gastrointestinal, hematological, and cardiovascular toxicities were predicted with a relatively high concordance, the ability to predict liver toxicities was much lower (80 versus 50% concordance, respectively). As a result, drug-induced liver injury (DILI) remains one of the leading causes of drug failure in the clinic and withdrawal from the market. The poor concordance for liver toxicity is driven by poor nonclinical to clinical translation for compounds that cause intrinsic dose-dependent liver toxicity, and by rare idiosyncratic events that occur in large patient trials or at post-marketing. Thus, one of the major challenges the pharmaceutical and biotechnology industries face is choosing whether to move a drug forward towards clinical testing based on animal safety data, specifically with respect to hepatotoxicity. In addition, to better understand and determine human risk, safety margins, and design safer drugs, the mechanism of action of drug toxicity should also be understood. Given the scale of this challenge and its negative impact on healthcare costs and development of new therapeutics, there is a critical need for more predictive and human relevant alternatives to animal models. Here, it was explored whether human microfluidic device culture technology, which has been shown to faithfully recapitulate the complex functions and pathophysiology of multiple human organs, may be used to build species-specific liver models that can be used to address this challenge.

### II. Study Details

i. Cell Sourcing

Cryopreserved primary human hepatocytes were purchased from Triangle Research Labs (Lonza, Morrisville, N.C., USA) and Gibco (Thermo Fisher Scientific, Waltham, Mass., USA); cryopreserved primary rat hepatocytes and freshly isolated dog hepatocytes were purchased from Biopredic (Saint Gregoire, France) and QPS (Newark, Del., USA), respectively. Cryopreserved primary human LSECs were purchased from Cell Systems (Kirkland, Wash., USA), rat and dog LSECs were purchased from Cell Biologics (Chicago, Ill., USA) and each were cultured according to their respective vendor protocols. Cryopreserved human and rat Kupffer cells were purchased from Thermo Fisher Scientific, human and rat stellate cells were purchased from Lonza, and each were cultured according to their respective vendor protocols. Dog Kupffer and stellate cells were isolated from hepatic NPC (QPS) at Emulate, Inc. following protocols by Olynyk et al. and Riccalton-Banks et al.

ii. Culturing Liver Cells in Microfluidic Devices

Prior to cell seeding, microfluidic devices, in this case S-I Chips (Emulate, Inc. Boston, Mass., USA), were functionalized using Emulate's proprietary protocols and reagents (ER™), including those described herein. After surface functionalization, both channels of the microfluidic device were coated with species-specific extracellular matrices (ECM). For the human bound microfluidic device, a mixture of collagen type I (Corning, Corning, N.Y., USA) and fibronectin, e.g. bovine, (Gibco) was used; for the rat bound microfluidic devices a mixture of collagen type IV, e.g. human placenta, (Sigma-Aldrich, St. Louis, Mo., USA) and fibronectin (Gibco) was used; for the dog bound microfluidic devices a mixture of collagen I (Corning), collagen type IV (Sigma-Aldrich), and fibronectin (Gibco) was used. Primary hepatocytes were seeded in the upper channel of the microfluidic devices at a concentration of 3.5 million cells/mL and later overlaid with Matrigel® (Corning), then incubated at 37° C., 5% CO2. For the dual-cell culture model (hepatocytes and endothelial cells), the LSECs were seeded at a concentration of 2-4 million cells/mL in the lower vascular channel.

For the quadruple-culture model (hepatocytes, endothelial cells, Kupffer cells, and stellate cells), a mixture of LSEC, Kupffer, and stellate cells were seeded in the channel of the microfluidic devices at the following concentrations: 3 million cells/mL for LSEC, 0.5 million cells/mL for LSEC, 0.1 million cells/mL for stellate cells. After cell seeding, the upper channel of the microfluidic device was maintained in William's E Medium (WEM) containing Glutamax (Gibco), ITS+ (Corning), dexamethasone (Sigma-Aldrich), ascorbic acid (Sigma-Aldrich), fetal bovine serum (FBS) (Sigma-Aldrich), and Penicillin/Streptomycin (Sigma-Aldrich). The vascular channel of the microfluidic device was maintained with species-specific endothelial media (Emulate, Inc.). Two days after seeding, the microfluidic devices were connected to the Human Emulation System™ (Emulate, Inc.), as described in part herein, and both of the microfluidic device channels were perfused at 30 μL/hr to provide a continuous supply of fresh media for the duration of the experiments.

iii. Immunofluorescence Staining

Microfluidic devices seeded with liver cells and static sandwich plate cultures were fixed with 4% paraformaldehyde for 15 minutes at room temperature, washed with PBS, and permeabilized (saponin 1% with 10% serum in PBS). Blocking and incubation with the primary antibodies (overnight in PBS containing 1% BSA or PBS containing 10% serum and 1% BSA) was followed by a two-hour incubation with secondary antibodies (Cell Signaling, Danvers, Mass., USA) in the same blocking buffer. Immunostaining was performed with specific primary antibodies (anti-MRP2, anti-stabilin-1, anti-BSEP, anti-α-SMA, anti-CD68; Abcam, Cambridge, Mass., USA) and images were acquired with either an Olympus fluorescence microscope (IX83) or Zeiss confocal microscope (AxiovertZ1 LSM880).

iv. Live Cell Staining

Microfluidic devices seeded with liver cells were stained in the upper channel with 5(6)-Carboxy-2′,7′-dichlorofluorescein diacetate (CDFDA) (ThermoFisher) to visualize bile canaliculi and MRP2 activity, cholyl-lysyl-fluorescein (CLF) (Corning) to visualize bile canaliculi and BSEP activity, Nile red (ThermoFisher) or AdipoRed (Lonza) to visualize lipid droplet accumulation, Tetramethylrhodamine, methyl ester (TMRM) (ThermoFisher) to visualize active mitochondria, and CellROX® (ThermoFisher) to visualize cellular oxidative stress. Each staining solution was prepared in blank medium or compound dosing medium and added to the upper channel, incubated for 15 minutes at 37° C., and washed three times with medium. The stained microfluidic devices were imaged using a specific filter, and were de-blurred with Olympus cellSens software. Using ImageJ-Fiji, the fluorescent channel images were histogram adjusted to remove background, followed by fluorescence quantification using the Integrated Density calculation function.

v. Exemplary Biochemical Assays.

Liver injury may include but is not limited to, drug induced liver injury (DILI), alcohol toxicity, obesity, diabetes, infection and/or hepatocellular carcinoma (HCC). Biological consequence or symptoms of such liver injury may include, but is not limited to, metabolic dysregulation, iron dysregulation (e.g., anemia/iron overload), carbohydrate imbalance, lipid imbalance, late onset diabetes, vitamin storage dysregulation, biliary tract damage, inflammation and/or fibrosis.

In some embodiments, assays are not limited to chips comprising human cells. Indeed, chips may comprise cells obtained from (as derived from) rat, dog, mouse, monkey, etc.

Albumin secretion from the upper channel was quantified using ELISA kits for human, rat, and dog models effluent samples (human and rat: Abcam; dog: Immunology Consultants Laboratory, Inc., Portland, Oreg., USA) and the assays were performed according to the protocol provided by each vendor.

Urea secretion into the upper channel was quantified using kits for human, rat, and dog effluent samples (Sigma and Abcam) and the assays were performed according to the protocol provided by the vendor.

Exemplary Biomarker, e.g. DILI: Keratin 18 levels were quantified in human model effluent samples from both the hepatocyte and vascular channels using the M65 EpiDeath CK18 ELISA Kit (DiaPharma, Detroit, Mich., USA). The assay was run following the vendor protocol using a standard curve ranging from 0-5,000 U/L. Additionally, a standard curve ranging from 0-500 U/L was generated independently for quality control.

Exemplary Biomarker, e.g. DILI: Alpha Glutathione S-Transferase (α-GST) levels were quantified in human model effluent samples from the upper channel using an ELISA kit (DiaPharma). The assay was run following the vendor protocol using a standard curve ranging from 0-64 μg/L. Additionally, a standard curve ranging from 0-50 μg/L was generated independently for quality control.

Exemplary Biomarker, e.g. DILI: Mir-122: Isolation of micro-RNAs was performed on upper and lower chamber eluates using Qiagen's (Hilden, Germany) miRNA isolation kit for serum and plasma. A total volume of 50 μL was used for isolation. Isolation was performed according to manufacturer's protocol using a 56.5 pM oligonucleotide spike-in control. Samples were eluted with 14 μL RNase-free water. Reverse transcription was performed using Thermo Fisher Scientific's TaqMan™ MicroRNA Reverse Transcription Kit according to the manufacturer's protocol. A total reaction volume of 15 μL was used. Real-time PCR was performed using Thermo Fisher Scientific's TaqMan™ Micro-RNA Assays according to manufacturer's protocol. A total reaction volume of 10 μL was used. Normalized rrCt was used for fold-change analysis.

Cytokines: Release of IL-6, MCP-1, and IP-10 from the vascular channel was quantified using U-PLEX biomarker human assays (Meso Scale Diagnostics, Rockville, Md., USA) and the assays were performed according to the protocol provided by the vendor.

Adenosine triphosphate (ATP) was measured using a modified CellTiter-Glo® Luminescent Cell Viability Assay (Promega, Madison, Wis., USA). Briefly 50 μL of cell lysate was mixed with 50 μL of ATP luminescent reaction mixture, incubate for 5 min, and luminescent intensity measured using a luminescence plate reader according to manufacturer's instruction. ATP concentrations in the lysates was quantified with an ATP standard curve.

Total glutathione was measured by adding, 50 μL of cell lysate from each sample to 50 μL of the GSH-GIo™ Reagent 2× (Promega) with DTT (˜2.5 mM final) reagent (to reduce glutathione) to each well of a 96-well plate. Samples were incubated at room temperature for 30 minutes. Reconstituted Luciferin Detection Reagent (100 μL) to each well of a 96-well plate. Mixed briefly on a plate shaker, incubated samples for 15 minutes and measured luminescence. Raw luminescent numbers were quantified to total glutathione (μM) by using a total glutathione standard curve.

AST, ALT, and GLDH concentrations in media from the upper channel were measured using the Siemens Advia 1800 Clinical Chemistry Analyzer (Tarrytown, N.Y.).

vi. CYP450 Enzyme Activity Measurement

CYP450 enzyme activity was determined using prototypical probe substrate compounds: phenacetin, midazolam, and bupropion (Sigma) as a cocktail or a single substrate, and cyclophosphamide and testosterone (Sigma) as a single substrate. For the cocktail substrate, a mixture of phenacetin at 30 midazolam at 3 μM, and bupropion at 40 μM final concentration respectively, was prepared in serum-free cell culture medium. For the single substrate, phenacetin at 100 μM, cyclophosphamide at 1 mM, and testosterone at 200 μM final concentration respectively, was prepared in serum-free cell culture medium. Enzyme activities were measured at Day 0, 3, 7, and 14 of hepatocyte cultures after a 30-min for testosterone, 1-hour and 2-hours incubation for the rest of the probe substrates under a flow rate of 200˜250 μL/hr of flow rate. The control condition was tested with hepatocytes in static sandwich monoculture plates using the same concentration of substrate and duration of incubation, using 500 uL in 24-well plate format. The reaction was stopped using acetonitrile with 0.1% formic acid, and formation of metabolites were measured using LC-MS.

vii. Gene Expression Analysis

In some embodiments, gene expression analysis is not limited to, PCR, RT-PCR, Taqman, etc.

In one embodiment, gene expression levels were analyzed using 2-step PCR. Total RNA isolation was performed using the RNeasy 96 Kit (Qiagen) or PureLink® RNA Mini Kit (Invitrogen) following the vendor protocol. To prepare cDNA, a combination of SuperScript VILO MasterMix or SuperScript™ IV (Invitrogen) and water was added to the isolated RNA samples and run on iCycler (BioRad) using the SuperScript VILO protocol or SimpliAmp Thermocycler (Applied Biosystems) using the SuperScript™ IV First-Strand Synthesis System protocol. Subsequent addition of a mixture of TaqMan Universal PCR MasterMix or TaqMan Fast Advanced MasterMix (ThermoFisher), water, and the appropriate probe was added to the cDNA samples and run on ViiA7 Real-Time PCR System (ThermoFisher) or QuantStudio™ 3 Real-Time PCR System (Applied Biosystems™) for qPCR analysis. Relative gene expression levels were calculated using ddCt method.

viii. APAP Metabolite Quantification

After APAP exposure to microfluidic devices seeded with human liver cells, effluent metabolite levels were quantified using LC-MS analysis. Relative quantitation was performed by first generating a standard curve using six concentrations of APAP standards: 0.78, 1.56, 3.12, 6.24, 12.48 and 24.96 mg/mL. These concentrations were plotted versus LC-MS peak area to generate a standard curve and subsequent linear regression equation: Y=0.5046.3*X. Effluent samples from Day 20 of culture were then analyzed, and the concentration of APAP present was interpolated using the standard curve regression: X=Y/5046.3*D, where X is the concentration of APAP, Y is the peak area from LC-MS, and D is the dilution factor. APAP-Glucuronide (APAP-Glu), the primary metabolite of APAP, was not quantified using a standard curve. Rather, the LC-MS peak area of APAP-Glu was reported corresponding to each sample.

ix. Image Analysis (e.g. CLF and CDFDA)

Both a brightfield and a fluorescence image was taken from each field of view. Both images were de-noised using median filtering, locally enhanced in contrast using the CLAHE algorithm, and thresholded to extract bright regions which corresponded to the biliary canaliculi (channel 1) and to CLF-labeled regions (channel 2), respectively, using Matlab (MathWorks, Natick, Mass., USA). CLF-labeled regions that most likely to corresponded to bile canaliculi were automatically identified based on geometric criteria and retained only if they co-localized with a bile canaliculi signal in channel 1. Specifically, the main criteria used to detect canalicular geometry were: (1a) Circularity is lower than 0.5 and eccentricity is greater than 0.8 (jagged. elongated canaliculi), or (1b) Eccentricity is greater than 0.8 and solidity is greater than 0.7 (smooth, elongated canaliculi), and (2) Total size is smaller than 70 μm2 and greater than 7 μm² (excluding noise and stained cell debris). These criteria were determined empirically using a small test set of images and then applied to images for the analysis. Circularity, eccentricity, and solidity were computed using Matlab built-in functions. The detected areas hence correspond to CLF-containing bile canaliculi. The data for treated and control samples were quantified by computing the area percentage of CLF-labeled bile canaliculi within each field of view (FOV). Because of the non-normal distribution of these data, statistical analysis was performed using the non-parametric Wilcoxon rank sum test. The total number of analyzed FOVs from 2 microfluidic devices each were n=8 FOVs for 30 μM bosentan and n=17 FOVs for vehicle-treated samples (0.1% DMSO).

FIGS. 6A-6E show recapitulation of species-specific drug toxicities in rat, dog, and human microfluidic liver devices. FIG. 6A is a schematic of a microfluidic device seeded with liver cells that recapitulates complex liver microarchitecture. Primary hepatocytes in the upper parenchymal channel in ECM sandwich format and NPCs (e.g. LSECs, Kupffer, and stellate cells) on the opposite side of the same membrane in the lower vascular channel. FIG. 6B shows albumin secretions after daily administration of bosentan at 1, 3, 10, 30, and 100 μM for 3 days in dual-cell (hepatocyte and LSECs) microfluidic devices seeded with human liver cells and plates (hepatocyte sandwich monoculture) and for 7 days in dual-cell dog and rat microfluidic device liver systems and plates (n=3 independent microfluidic devices and plate wells). FIG. 6C shows quantification of % CLF-positive area in bile canaliculi (BC) from the parenchymal channel after bosentan treatment at 30 μM for 7 days in microfluidic devices seeded with human liver cells. Mann-Whitney U test (n=3 independent microfluidic devices with 3 randomly selected different areas per microfluidic device, see detailed description on the analysis herein). FIG. 6D shows representative images of CLF (green, BSEP substrate) and BSEP (red, DAPI in blue) from the parenchymal channel. FIG. 6E shows quantification of BSEP-positive area and fold change of BSEP gene expression. Mann-Whitney U test (n=3 independent microfluidic devices). Scale bar, 20 μm. *P<0.05, **P<0.01, ****P<0.0001. Error bars present mean±SEM.

x. Bile Salt Export Pump (BSEP) Area Quantification

The fluorescent channel images were first de-blurred with Olympus cellSens software. Using ImageJ, they were then histogram adjusted to remove background, adaptive thresholded to extract fluorescent regions (a plugin developed by Qingzong Tseng using the adaptive threshold method of the OpenCV library), and analyzed using the built-in 3D Object Counter function with an area cutoff value to remove noise. N=3 FOVs from each microfluidic device were analyzed for vehicle (0.1% DMSO) and 30 μM bosentan conditions.

xi. Lipid Accumulation Quantification

The stained microfluidic devices were imaged using the TRITC filter, and were de-blurred with Olympus cellSens software. Using ImageJ-Fiji, the fluorescent channel images were histogram adjusted to remove background, followed by fluorescence quantification using the Integrated Density calculation function. N=5 FOVs per microfluidic device were analyzed for conditions: vehicle (0.1% DMSO), FIAU dosing groups, and MTX dosing groups.

xii. Kupffer Cell and Activated Stellate Cell Count

The number of CD68- or α-SMA-positive cells in the vascular channel in each group was counted in each field of view (360 mm²) and quantified.

xiii. Alphα-SMA Intensity Quantification

The fluorescent channel (588) was first background subtracted and processed using the median filter of Image J. After processing, the signal was thresholded using adaptive thresholding function of Image J (Otsu method) to extract fluorescent regions and to measure the relative intensity of the fluorescent signal. N=4 to 6 FOVs per microfluidic device from each microfluidic device were analyzed. Results were normalized to the relative control condition (vehicle, DMSO 0.1%) and reported as fold increment in respect to control.

xiv. Glycogen Quantification Assay

Samples, e.g. cell lysate, dilution range 1:500 to 1:1000. Recommended assay flow rate (Liver-Chip) 30 uL/h. Example, use a Glycogen Assay Kit (Abeam, ab65620); run assay as described on vendor site.

xv. Cholesterol Quantification Assay

Hepatocyte medium composition adjustment: For best results, use hepatocyte media without FBS and with ITSG (ThermoFisher #41400045), instead of the ITS premix that is used as complete Liver-Chip media.

Samples, e.g. effluent collected from liver chip, recommended effluent dilution (Liver-Chip), either no dilution, or up to 1:10 (at flow rate 30 μL/h). Example, use a Cholesterol Assay Kit (Abeam #: A12216); run assay as described on vendor site. Note: Cholesterol concentration of media may change assay dilution range. Adjust accordingly. Use vendor fluorometric protocol for best results.

Sample quantification Recommended:
Chol_{Net}=Chol_{effluent}−Chol_{dosing media}
Chol_{Net}=Net effluent cholesterol (μg/mL)
Chol_{effluent}=Cholesterol from effluent (μg/mL)
Chol_{dosing media}=Cholesterol from dosing media (μg/mL)
   xvi. Triglycerides Quantification Assay

Samples, e.g. effluent collected from liver chip, recommended effluent dilution (Liver-Chip), 1:5 (at flow rate 30 μL/h). Example, use a Triglyceride Assay Kit (Abeam ab65336); run assay as described on vendor site. Use vendor fluorometric protocol for best results. Sample quantification Recommended:
TGₙₑₜ=TG_{effluent}−TG_{treatment media}
TG_{Net}=net effluent triglycerides (μg/mL)
TG_{effluent}=triglycerides from effluent (μg/mL)
TG_{treatment media}=triglycerides from treatment media (μg/mL)
   xvii. Hepcidin Quantification Assay.

Hepcidin (hepcidin antimicrobial peptide) refers to a peptide regulator of iron metabolism, synthesized and secreted by hepatocytes and other cell types. As an iron-regulatory hormone, hepcidin regulates intestinal iron absorption, plasma iron concentrations, and tissue iron distribution by inducing degradation of its receptor, the cellular iron exporter ferroportin. Ferroportin exports iron into plasma from absorptive enterocytes, from macrophages that recycle the iron of senescent erythrocytes, and from hepatocytes that store iron. Increased hepcidin concentrations in plasma are pathogenic in iron-restrictive anemias including anemias associated with inflammation, chronic kidney disease and some cancers. Hepcidin deficiency causes iron overload in hereditary hemochromatosis and ineffective erythropoiesis. Hepcidin, ferroportin and their regulators represent potential targets for the diagnosis and treatment of iron disorders and anemias. Hepcidin binds to a receptor/iron exporter ferroportin and causes its internalization and degradation. Major iron disorders are caused by dysregulation of hepcidin. Molecular analysis of the hepcidin-ferroportin system allows targeting for diagnosis and therapy.

Quantify Hepcidin from Emulate Organ-Chip Effluent.

Hepcidin levels may change depending on cell injury status or based on donor-to-donor variability. Therefore, sample dilutions may need to be modified to accommodate different experimental conditions or cells from different donors.

Effluent Sampling: Human Hepcidin Quantikine ELISA Kit (R&D Systems #DHP250). Run assay as described on supplier site (www.rndsystems.com/products/human-hepcidin-quantikine-elisa-kit_dhp250).

xviii. Iron Dysregulation (e.g., Anemia/Iron Overload).

In some embodiments, hepcidin may be regulated by iron levels on chip, and the dysregulation of this process may mimic iron disorders. Hepcidin also appears to block, at least partially, the export of stored iron from hepatocytes. Thus, in some embodiments, a combination of changing iron levels in fluids contacting cells on chips, with altered hepcidin levels, may be used for mimicking iron dysregulation. In some embodiments, a compound may be added for altering hepcidin production or secretion levels or iron levels in hepatocytes on chips.

xix. Semi-Quantitation of TAK-875 Metabolites from Human Liver-Chip

Incubation of ¹⁴C-TAK-875 with human liver microsomes in the presence of NRS and UDPGA/UDPAG: The goal of the in vitro incubation is to generate ¹⁴C-TAK-875 acyl glucuronide and ¹⁴C-M1 metabolite for quantitation of these 2 metabolites in incubates from human Liver-Chip model. Human liver microsomes (HLM, 1 mg/mL, BD Gentest, Franklin Lakes, N.J., USA) was pre-incubated with 10 μM 14 C-TAK-875 at 37° C. for 3 minutes. The NADPH regenerating system and uridine 5′-diphosphoglucuronic acid (UDPGA, 5 mM): uridine 5′-diphospho-N-acetylglucosamine (UDPAG, 1mM) were added to initiate the reaction at the end of pre-incubation. The reaction mixtures were further incubated for 60 minutes. Incubates in the absence of radiolabeled TAK-875 served as calibration matrices for samples from Liver-Chip.

The reaction was quenched with 5 volumes of acetonitrile:isopropyl alcohol/1:1 fortified with 0.1% formic acid and ammonium formate (500 mM, pH 3.0) to stabilize the acyl glucuronides.

The mixture was vortexed mixed and sonicated prior to centrifugation at 3000 g for 10 minutes at 4° C. The resulting supernatants were dried under nitrogen. The dried residues were suspended in 300 μL of acetonitrile:water:isopropyl alcohol/1:2:1 fortified with 0.1% formic acid. The suspension was filtered through 0.45 μm Nylon filters for LC/RAD/MS analysis.

xx. Preparation of Samples for Quantitation by Calibration of MS Response:

An equal volume of 14 C-TAK-875 HLM incubate and sample matrices (vehicle-treated) from Liver-Chip were mixed prior to analysis. Similarly, samples from Liver-Chip following treatment with cold TAK-875 at 10 μM for ˜2 weeks were mixed with the HLM matrices (without ¹⁴C-JNJ-TAK-875) prior to analysis.

The unchanged drug, M1 and acyl glucuronide metabolites of TAK-875 in eluates from Liver-Chip were quantified using the peak areas from ¹⁴C and MS of HLM incubate according to the equation as follows: (¹⁴C-Peak Area/Peak area of same component from MS ionization of ¹⁴C sample)*Peak area of same component from MS ionization of samples.

xxi. Statistical Analysis

As indicated in the FIG. legends, one-way ANOVA, Sidak's and Dunnett's multiple comparisons test was used for parametric data and the Mann-Whitney U test or Kruskal-Wallis tests was used for nonparametric data. Statistical analyses were performed using Prism 7 (GraphPad).

xxii. Testing of JNJ-1 in Rats and Dogs

JNJ-1, prepared in purified water, was administered once daily for 14 days to 5 male and 5 female rats at doses of 5, 25, and 125 mg/kg. In a separate study, JNJ-1, also in purified water, was administered once daily for 4 weeks to 5 male and 5 female dogs at 0 or 40 mg/kg/day, and to 3 males and 3 female dogs at 2 or 10 mg/kg/day. Mortality, clinical observations, body weight, food consumption, clinical pathology, gross necropsy and microscopic examination of selected tissues, and toxicokinetics were evaluated. Rats or dogs were fasted overnight prior to blood collection for measurement of clinical pathology parameters (including AST and ALT) at the end of 14 days (rats) or 4 weeks (dogs) of dosing. Histology slides (hematoxylin and eosin staining and Mason's Trichrome staining) of the liver tissues were prepared and evaluated microscopically. The quantification of JNJ-1 was conducted using a qualified liquid chromatographic-triple quadrupole mass spectrometric (LC-MS/MS) procedure.

A double-blind, placebo-controlled, randomized, dose-escalating, sequential group design was conducted in 2 Parts. Subjects received daily doses of JNJ-28312141 or placebo for a total of 2 weeks (the first dose on Day 1 and then daily on Days 3 to 14). Part 1 included 5 cohorts of healthy male or female subjects. In Part 2 of the study, 2 cohorts of postmenopausal women (PMW) were enrolled. Within each cohort of 12 subjects, 9 subjects were randomized to JNJ-1, and 3 subjects were randomized to placebo. Dose escalation only occurred after the Sponsor and the Investigator had performed a satisfactory review of the preliminary safety data including those obtained on the post-study follow up visit (anytime between Days 30 to 32), and of the drug concentration and PK data (if available) at the current dose level. In addition to efficacy endpoints, and pharmacokinetics, an assessment of safety included measurement of clinical chemistry parameters, including ALT and AST.

### Testing of JNJ-2 in Rats

Following the discovery of liver fibrosis in a 14-day male rat study with JNJ-2, a mechanistic study was performed with the same compound at the high dose, three time points (3, 7, and 14 days), 12 male rats per group (6 euthanized at the end of treatment, and 6 after a 14-day recovery period). Regarding histology procedures, the liver was sampled. Briefly, 5 serial sections were prepared. One was stained with hematoxylin and eosin (H&E) kit. The 4 others were submitted to histochemical and immunohistochemical stains. Collagen was detected with the Van Gieson kit (Merck, Darmstadt, Germany), and reticulin, with the Reticulum kit (Sigma-Aldrich). A monoclonal mouse anti-human aSMA antibody (Dako, Denmark) was incubated for 2 hours at room temperature after antigen retrieval, endogenous peroxidase block and with normal goat serum block, and revealed with the detection kit Vectastain ABC Elite (Vector Labs, Burlingame, Calif., USA) and the chromogen DAB (Dako). A monoclonal mouse anti-rat CD68 (ED1) antibody (Serotec, Raleigh, N.C., USA) followed the same protocol.

### Testing of JNJ-3 in Dogs

JNJ-3 (in 20% hydroxypropyl-β-cyclodextrin) was administered once daily for 14 days to 3 male beagle dogs per group at doses of 15 and 65 mg/kg. Mortality, clinical observations, body weight, food consumption, clinical pathology, gross necropsy and microscopic examination of selected tissues, and toxicokinetics were evaluated. Dogs were fasted overnight prior to blood collection for measuring clinical pathology parameters (including AST and ALT) after 6 and 14 days of dosing. Histology slides (hematoxylin and eosin staining and Mason's Trichrome staining) of the liver tissues were prepared and evaluated microscopically. The quantification of JNJ-3 was conducted using a qualified liquid chromatographic-triple quadrupole mass spectrometric (LC-MS/MS) procedure.

III. Recapitulation of Species-Specific Drug Toxicities in Microfluidic Devices Seeded with Rat, Dog, and Human Cells

Species-specific microfluidic devices lined by living rat, dog, or human hepatic cells were constructed using dual channel microfluidic devices that have previously been shown to recapitulate the multicellular architecture, tissue-tissue interfaces, vascular perfusion, interstitial fluid flow, and the relevant physical microenvironment of multiple human organs, including lung, intestine, and kidney. Primary rat, dog or human hepatocytes were cultured in the upper parenchymal channel within an extracellular matrix (ECM) sandwich on top of an ECM-coated, porous membrane that separates the two parallel microchannels, and relevant species-specific rat, dog, or human liver sinusoidal endothelial cells (LSECs), with or without liver Kupffer cells and/or stellate cells, were cultured on the opposite side of the same membrane in the lower vascular channel (FIG. 6A). These studies were initiated by analyzing dual-cell microfluidic devices liver systems containing only the hepatocytes and LSECs (FIG. 12A), which revealed that three species of primary hepatocytes formed characteristic branched bile canalicular networks lined by functional multidrug resistance-associated protein 2 (MRP2) efflux transporters and maintained their stereotypical in vivo-like liver epithelial morphologies for at least 14 days in culture when co-cultured with liver endothelium under continuous flow (FIG. 12B). In contrast, the same human, dog, and rat hepatocytes failed to form well developed bile canaliculi when maintained for 2 weeks without endothelium in a static ECM sandwich culture plates (FIG. 12B). In the vascularized microfluidic device seeded with liver cells, the underlying LSECs also displayed the multifunctional scavenger receptor stabilin-1, which is expressed selectively on sinusoidal endothelial cells of liver, spleen, and lymph nodes (FIG. 12B).

FIGS. 6A-6E show recapitulation of species-specific drug toxicities in rat, dog, and human microfluidic liver devices. FIG. 6A is a schematic of a microfluidic device seeded with liver cells that recapitulates complex liver microarchitecture. Primary hepatocytes in the upper parenchymal channel in ECM sandwich format and NPCs (e.g. LSECs, Kupffer, and stellate cells) on the opposite side of the same membrane in the lower vascular channel. FIG. 6B shows albumin secretions after daily administration of bosentan at 1, 3, 10, 30, and 100 μM for 3 days in dual-cell (hepatocyte and LSECs) microfluidic devices seeded with human liver cells and plates (hepatocyte sandwich monoculture) and for 7 days in dual-cell dog and rat microfluidic device liver systems and plates (n=3 independent microfluidic devices and plate wells). FIG. 6C shows quantification of % CLF-positive area in bile canaliculi (BC) from the parenchymal channel after bosentan treatment at 30 μM for 7 days in microfluidic devices seeded with human liver cells. Mann-Whitney U test (n=3 independent microfluidic devices with 3 randomly selected different areas per microfluidic device, see detailed description on the analysis herein). FIG. 6D shows representative images of CLF (green, BSEP substrate) and BSEP (red, DAPI in blue) from the parenchymal channel. FIG. 6E shows quantification of BSEP-positive area and fold change of BSEP gene expression. Mann-Whitney U test (n=3 independent microfluidic devices). Scale bar, 20 μm. *P<0.05, **P<0.01, ****P<0.0001. Error bars present mean±SEM.

To assess the physiological function of the dual-cell microfluidic device liver systems, secretion of two major hepatocyte products: albumin and urea, were measured and compared to results obtained from the same human, rat, and dog hepatocytes cultured alone in the static sandwich culture plates. These studies revealed that three species-specific microfluidic devices seeded with liver cells maintained significantly higher (4- to 14-fold greater) levels of these liver-specific functions than cells in conventional sandwich monocultures (FIG. 12C). The quantitative range of albumin production measured in the microfluidic devices seeded with human liver cells of ˜40-60 μg/day/million cells (between days 7 and 14) is very similar to that estimated for humans in vivo (50 μg/day/million cells) using in vitro-to-in vivo extrapolation (iViVE) techniques. In contrast, hepatocytes within conventional sandwich plates showed significantly lower (7.8- to 8.5-fold lower) levels of albumin production than cells in the microfluidic device seeded with human liver cells over the same time period.

FIGS. 12A-12C show morphology and functionality of species-specific dual-cell microfluidic device liver systems. FIG. 12A shows a schematic of the dual-cell microfluidic device liver system that recapitulates complex liver microarchitecture. Primary hepatocytes in the upper channel in ECM sandwich format and LSECs on the opposite side of the same membrane in the lower vascular channel. FIG. 12B shows representative images of hepatocytes (bright-field), CDFDA (green) to visualize bile canaliculi in hepatocytes, MRP2 (green and DAPI in blue) in hepatocytes, and stabilin-1 (red and DAPI in blue) in LSECs after 14 days of culture in human, dog, and rat microfluidic device liver systems and sandwich monoculture plates. Scale bar, 100 μm. FIG. 12C shows albumin and urea secretions in human, dog, and rat microfluidic device liver systems over 2 weeks compared to static sandwich monoculture plates. Dunnett's multiple comparisons test (n=7-20 independent microfluidic devices, n=3-9 independent wells in plate). **P<0.01, ***P<0.001 , ****P<0.0001. Error bars present mean±SEM.

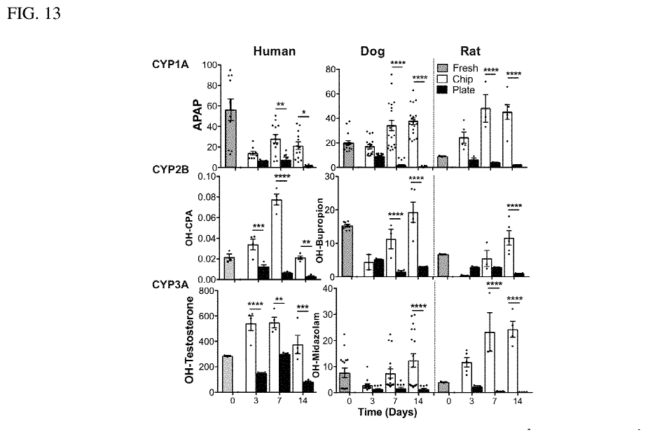

To further evaluate the physiological relevance of the dual-cell microfluidic devices seeded with liver cells, the drug metabolizing capacity of the hepatocytes was characterized by measuring activities of multiple cytochrome P450 (CYP) isoforms (CYP1A, CYP2B, and CYP3A) that represent CYP families involved in drug metabolism with a substrate cocktail approach using concentrations of their respective substrates (phenacetin, bupropion, and midazolam) or single substrate (cyclophosphamide for CYP2B and testosterone for CYP3A for human model) that mirror their Michaelis constant (Km) in humans. These three isoforms also represent the major CYPs regulated by the xenosensors: aryl hydrocarbon receptor (AhR), constitutive androstane receptor (CAR), and pregnane X receptor (PXR). These studies revealed that CYP activities measured in the dual-cell human, rat, and dog microfluidic devices seeded with liver cells during the 14-day culture period were comparable to, or in some cases greater than, those exhibited by freshly isolated hepatocytes (FIG. 13), which are the gold-standard model currently used by pharmaceutical researchers. In contrast, there was a significant decline in CYP activities in three species in sandwich monoculture plates over the same time period (FIG. 13).

FIG. 13 shows cytochrome P450 enzyme activity in species-specific dual-cell microfluidic device liver systems. Cytochrome P450 enzyme activity in human, dog, and rat microfluidic device liver systems compared to conventional sandwich monoculture plates and fresh hepatocyte suspension over 2 weeks using a cocktail (for dog and rat) or single (for human) probe substrate. Unit: pmol/min/10⁶ cells. Dunnett's or Sidak's multiple comparisons test (n=3 to 20 independent microfluidic devices). *P<0.05, **P<0.01, ***P<0.001 , ****P<0.0001. Error bars present mean±SEM.

To explore whether these dual-cell microfluidic devices seeded with liver cells could be used to predict species-specific DILI responses, the three species models were used to evaluate hepatotoxic effects induced by bosentan, which is a dual endothelin receptor antagonist that causes cholestasis in humans, but not in rats or dogs, by inhibiting the bile salt export pump (BSEP) and inducing hepatocellular accumulation of bile salts. Daily administration of bosentan at 1, 3, 10, 30, and 100 μM resulted in decreases in albumin secretion with different potencies in these species-specific microfluidic devices seeded with liver cells, with an IC50 of 10, 30 and >100 μM in human, dog, and rat microfluidic devices, respectively (FIG. 6B). Notably, the potency in human microfluidic liver systems approximated plasma concentrations of bosentan (Cmax=7.4 μM) that has been associated with DILI in humans, and the model was more sensitive in detecting bosentan toxicity compared to sandwich monoculture plates (FIG. 6B) or other complex in vitro liver models, such as 3D human spheroid hepatic cultures where the IC50 was found to be more than 10-fold higher. Co-treatment of bosentan (30 μM) with cholyl-lysyl-fluorescein (CLF), a BSEP substrate, resulted in more than a 50% reduction in its efflux (FIG. 6C) and resultant intracellular accumulation of CLF (FIG. 6D) in human hepatocytes. Inhibition of BSEP activity also was accompanied by decreases in BSEP protein and mRNA levels ((FIG. 6E), reflective of an adaptive response secondary to transporter inhibition. Thus, in addition to mimicking species-specific hepatotoxicities in vitro, these results illustrate that mechanisms of DILI that involve hepatic transporters can be studied in the microfluidic device liver system, and they highlight the advantage of the microfluidic device liver system in terms of integrating mechanisms of toxicity (BSEP inhibition) to functional outcome (decrease in albumin synthesis) in the same model.

FIGS. 6A-6E show recapitulation of species-specific drug toxicities in rat, dog, and human microfluidic liver devices. FIG. 6A is a schematic of a microfluidic device seeded with liver cells that recapitulates complex liver microarchitecture. Primary hepatocytes in the upper parenchymal channel in ECM sandwich format and NPCs (e.g. LSECs, Kupffer, and stellate cells) on the opposite side of the same membrane in the lower vascular channel. FIG. 6B shows albumin secretions after daily administration of bosentan at 1, 3. 10, 30, and 100 μM for 3 days in dual-cell (hepatocyte and LSECs) microfluidic devices seeded with human liver cells and plates (hepatocyte sandwich monoculture) and for 7 days in dual-cell dog and rat microfluidic device liver systems and plates (n=3 independent microfluidic devices and plate wells). FIG. 6C shows quantification of % CLF-positive area in bile canaliculi (BC) from the parenchymal channel after bosentan treatment at 30 μM for 7 days in microfluidic devices seeded with human liver cells. Mann-Whitney U test (n=3 independent microfluidic devices with 3 randomly selected different areas per microfluidic device, see detailed description on the analysis herein). FIG. 6D shows representative images of CLF (green, BSEP substrate) and BSEP (red, DAPI in blue) from the parenchymal channel. FIG. 6E shows quantification of BSEP-positive area and fold change of BSEP gene expression. Mann-Whitney U test (n=3 independent microfluidic devices). Scale bar, 20 μm. *P<0.05, **P<0.01, ****P<0.0001. Error bars present mean±SEM.

IV. Detection of More Complex Hepatotoxicities Using Quadruple-Cell Microfluidic Devices Seeded with Liver Cells.

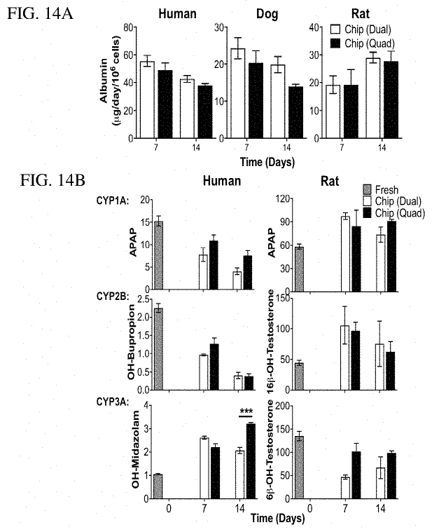

To add higher order organ complexity to the microfluidic devices seeded with liver cells necessary to study a wider range of hepatoxicities, species-specific, non-parenchymal (NP), liver stellate and Kupffer cells were integrated into the vascular channel to develop the quadruple-cell microfluidic device liver system (FIG. 6A). These species-specific, quadruple-cell microfluidic device liver systems also exhibited high levels of albumin secretion similar to those observed in the dual-cell microfluidic devices (FIG. 14A), and the human and rat microfluidic devices maintained high levels of CYP enzyme activities that were similar to, or higher than, those observed in freshly isolated hepatocytes or the dual-cell microfluidic devices (FIG. 14B).

FIGS. 14A-14B show a comparison of hepatic functionalities between dual- and quadruple-cell microfluidic device liver systems. FIG. 14A shows a comparison of albumin secretions between dual- and quadruple-cell microfluidic device liver systems from three species models. FIG. 14B shows a comparison of CYP450 enzyme activities between dual- and quadruple-cell microfluidic devices seeded with either rat or human liver cells. Dunnett's or Sidak's multiple comparisons test (n=3 to 4 independent microfluidic devices). ***P<0.001. Error bars present mean±SEM.

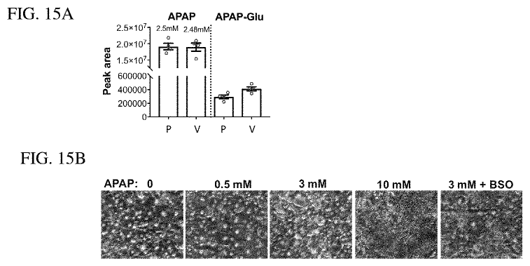

The generic analgesic, acetaminophen (APAP), can produce DILI resulting in whole organ failure and death when over-dosed. APAP toxicity is mediated by formation of the toxic and reactive metabolite NAPQI that depletes cellular glutathione (GSH) causing oxidative stress, and the drug also can be detoxified by hepatocytes resulting in formation of glucuronide and sulfate metabolites. To evaluate APAP toxicity in the human quadruple-cell microfluidic device liver systems, a constant flow rate was maintained that was determined based on known intrinsic clearance to best reproduce its metabolism rate and turnover (10 μL/hr of flow rate). Metabolism of APAP microfluidic devices was confirmed by detection of significant amounts of APAP glucuronide in both the parenchymal and vascular channels following daily administration of 3 mM APAP for 20 days (FIG. 15A), which confirmed that four cell types were exposed to the hepatocyte-derived metabolites as a result of diffusion through the porous membrane. FIGS. 15A-15B show detection of glucuronide metabolites of APAP and hepatocellular injury using quadruple-cell human microfluidic device liver systems. FIG. 15A shows APAP glucuronide metabolites formation from upper parenchymal (P) and lower vascular (V) channels after APAP treatment at 3 mM for 20 days from microfluidic devices seeded with human liver cells. (n=4 independent microfluidic devices). FIG. 15B shows representative bright-field images of hepatocytes after daily administration of APAP at 0.5, 3, and 10 mM and co-administration of APAP 3 mM and 200 μM of buthionine sulfoximine (BSO) for 7 days in microfluidic devices seeded with human liver cells.

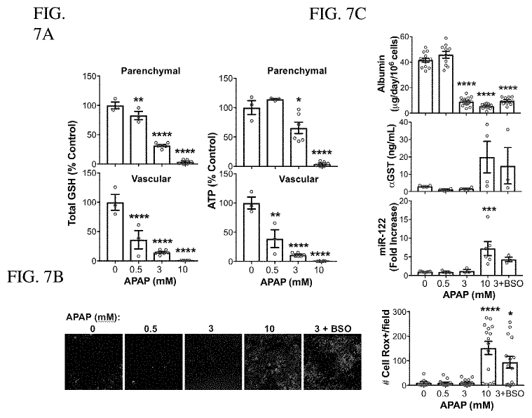

Treatment with APAP resulted in dose-dependent depletion in total GSH and ATP at all concentrations tested (0.5, 3, and 10 mM) in both the hepatocytes within the parenchymal channel, and even more potently in the NPCs in the vascular channel (FIG. 7A), highlighting that APAP toxicity is not limited to liver epithelial cells. The depletion of GSH also is suggestive of formation of reactive oxygen species (ROS), which was confirmed by measuring their levels using a fluorescent reporter assay (FIG. 7B), and thus, the observed increased sensitivity of NPCs may be due to a reduced detoxification capacity relative to that in hepatocytes. Early APAP-induced depletion of GSH and ATP depletion also was followed by a decline in hepatocyte morphology (FIG. 7B) and function, as measured by decreased albumin synthesis and oxidative stress-related injury markers, such as alpha glutathione S-transferase (α-GST) and microRNA 122 (miR122) (FIG. 7C). In addition, co-treatment of a moderate dose of APAP (3 mM) with the glutathione depleting agent, buthionine sulfoximine (BSO; 200 μM), increased sensitivity to APAP toxicity based on increased release of ROS (FIG. 7B), as well as miR122 and α-GST (FIG. 7C) that were not detected in the absence of BSO, further confirming the reported role of ROS in APAP-induced hepatotoxicity.

FIGS. 7A-7C shows detection of hepatocellular injury and release of various DILI biomarkers using quadruple-cell human microfluidic device liver systems. FIG. 7A shows total GSH and ATP levels from the parenchymal and vascular channels after daily administration of APAP at 0.5, 3, and 10 mM for 7 days in microfluidic devices seeded with human liver cells. FIG. 7B shows representative images of ROS levels (magenta, CellROX) after daily administration of APAP at 0.5, 3, and 10 mM and co-administration of 3 mM of APAP and 200 μM of BSO for 7 days in microfluidic devices seeded with human liver cells and quantification of number of CellROX-positive events per field of view. Kruskal-Wallis tests (n=3 independent microfluidic devices with 3 to 5 randomly selected different areas per microfluidic device). Scale bar, 100 μm. FIG. 7C shows albumin, αGST, and miR-122 secretions from the parenchymal channel after APAP treatment for 7 days in microfluidic devices seeded with human liver cells. Dunnett's multiple comparisons test (n=10-18 independent microfluidic devices for albumin, n=3˜9 independent microfluidic devices for the rest). *P<0.05, **P<0.01, ***P<0.001, ****P<0.0001. Error bars present mean±SEM.

To explore whether the quadruple-cell microfluidic devices could be used to model more complex liver DILI mechanisms that target Kupffer cells, and a JNJ compound was studied, which is a cFMS/FLT3 inhibitor that was discontinued in Phase I clinical trials due to elevation of the transaminases, alanine aminotransferase (ALT) and aspartate aminotransferase (AST), in 3/12 and 7/12 healthy subjects, respectively. Dose-related elevation in transaminases were also observed in rat and dog studies, but without any correlative microscopic changes in the liver, and this toxicity is suspected to be due to Kupffer cell depletion and subsequent increase in the half-life of the transaminases. Interestingly, administration of a JNJ compound in microfluidic devices seeded with human liver cells resulted in Kupffer cell depletion, as indicated by decreased number of CD68-positive cells, a dose-dependent decrease in interleukin 6 (IL-6) and monocyte chemoattractant protein-1 (MCP-1) in the vascular channel. Results demonstrated the ability of the human quadruple-cell microfluidic devices seeded with liver cells to detect mechanisms that target Kupffer cells, independently of hepatocytes.

### V. Modeling Steatosis and Fibrosis in Microfluidic Devices

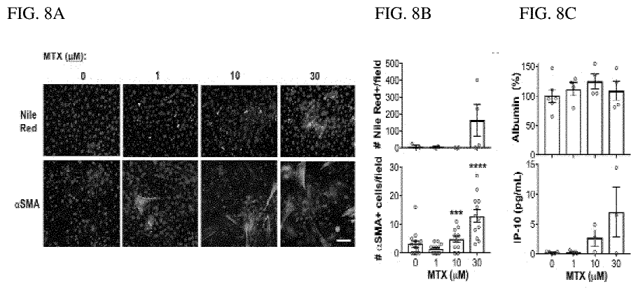

Methotrexate (MTX) causes liver injury in humans characterized by steatosis, stellate cell hypertrophy and fibrosis at maximal plasma concentrations of ˜1 μM in some patient populations. These findings were recapitulated in the quadruple-cell microfluidic device liver systems where daily administration of MTX at 1, 10, and 30 μM for 7 days resulted in microscopic evidence of lipid accumulation as detected by Nile red, and stellate cell activation as indicated by increased expression of the fibrosis marker, α-smooth muscle action (α-SMA) (FIG. 8A, 8B). These changes were also associated with increases in interferon y-induced protein 10 kDa (IP-10), a chemokine whose elevation is associated with liver inflammation and fibrosis although there were no abnormalities in albumin secretion (FIG. 8C) or hepatocyte morphology (not shown), which is consistent with lack of predictive or diagnostic biomarkers for monitoring these toxicities in humans. These studies suggest that inclusion of microscopic endpoints for steatosis and fibrosis in the quadruple-cell microfluidic device liver systems could be an approach to identify compounds with potential risk for these toxicities.

FIGS. 8A-8C shows detection of Kupffer cell depletion, steatosis and fibrosis in microfluidic devices seeded with human liver cells. FIG. 8A shows representative images of lipid droplets (yellow, Nile red and DAPI in blue) from the parenchymal channel and alpha-SMA (green) from the vascular channel to indicate activated stellate cells after daily administration of MTX at 1, 10, and 30 μM for 7 days in microfluidic devices seeded with human liver cells. FIG. 8Bshows quantification of Nile red-positive events per field of view and α-SMA-positive cells per field of view. Kruskal-Wallis tests (n=3 independent microfluidic devices with 3-5 randomly selected different areas per microfluidic device). FIG. 8C shows albumin secretion from the parenchymal channel and IP-10 secretion from the vascular channel after MTX treatment for 7 days and 1 day respectively in microfluidic devices seeded with human liver cells. Scale bar, 100 μm. *P<0.05, **P<0.01, ***P<0.001, ****P<0.0001. Error bars present mean±SEM.

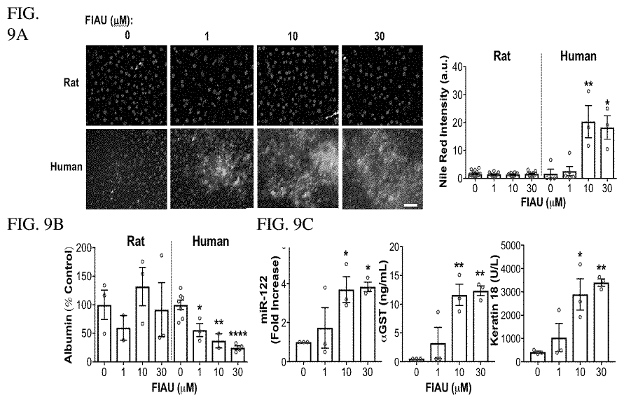

To investigate whether cross-species microfluidic devices liver models could be used to predict human-specific steatosis, fialuridine (FIAU) was tested in rat and human quadruple-cell microfluidic device liver systems. Development of FIAU, an anti-viral nucleoside analog, was discontinued in Phase II clinical trials due to liver failure and deaths in 5/15 patients, caused by microvesicular steatosis. A review of the animal toxicology data concluded that the studies could not have predicted severe liver injury caused by FIAU. Daily administration of FIAU at 1, 10, and 30 μM for 10 days in the microfluidic devices seeded with human cells resulted in a dose-dependent increase in lipid accumulation (FIG. 9A). There also was a concomitant dose-dependent decline in albumin secretion at concentrations ≥1 μM, and release of liver injury markers including miR122, α-GST, and keratin 18 (K-18) (FIG. 9B and FIG. 9C). In contrast, there were no effects on lipid accumulation or hepatocyte function following treatment of microfluidic devices seeded with rat liver cells with FIAU at the same concentrations and treatment duration as the microfluidic devices seeded with human liver cells (FIG. 9A and FIG. 9B), which is consistent with past preclinical studies.

FIGS. 9A-9C shows a comparison of species differences in steatosis using rat and human microfluidic device liver systems following fialuridine (FIAU) treatment. FIG. 9A shows representative images of lipid droplets (yellow, Nile red and DAPI in blue) from the parenchymal channel after daily administration of FIAU at 1, 10, and 30 μM for 10 days in rat and human microfluidic device liver systems and quantification of Nile red intensity. FIG. 9B shows albumin secretions as % control after FIAU treatment for 7 days in rat and human microfluidic device liver systems. FIG. 9C shows Mir-122, alpha-GST, and keratin 18 secretions after FIAU treatment for 10 days in microfluidic devices seeded with human liver cells. Dunnett's multiple comparisons test (n=3 independent microfluidic devices). Scale bar, 100 μm. *P<0.05, **P<0.01, ****P<0.0001. Error bars present mean±SEM.

VI. Use of Species-Specific Microfluidic Devices Seeded with Liver Cells to Query Human Relevance of Animal Liver Toxicities

It is not uncommon for compounds to be discontinued due to liver toxicity observed in rats or dogs prior to testing in humans because of uncertainties on the human relevance of these findings. To evaluate whether species-specific microfluidic devices seeded with liver cells could be used to assess human relevance, a Janssen proprietary compound that was discontinued due to liver toxicity in rats was characterized in the cross-species microfluidic device liver systems. Daily oral administration of a JNJ compound to rats for 2 weeks resulted in liver fibrosis, supported by increased α-SMA staining within stellate cells that was persistent 3 months after compound wash-out. These findings were associated with chronic inflammation of portal areas and decreases in albumin with no changes in transaminases in rats, and as a result, this compound was discontinued prior to testing in non-rodent species. However, daily treatment of of this compound at different concentrations in rat quadruple-cell microfluidic device liver systems for 4 days resulted in a dose-dependent increase in expression of α-SMA specifically within stellate cells, whereas treatment of microfluidic devices seeded with human liver cells at the same concentrations did not produce these abnormalities, even when extended for 14 days of treatment. These results imply that a compound might be dropped from development based on results of preclinical studies that were not indicative of human responses.

Also tested was another Janssen proprietary compound, that was discontinued from further development due to hepatocellular necrosis and biliary hyperplasia following daily dosing in dogs. These findings were associated with significant elevations of ALT and AST at the maximal plasma concentrations of 19.4 μM. Daily administration for 24 hours significantly decreased albumin secretion at ≥1 μM in microfluidic devices seeded with dog liver cells, whereas this was only observed at doses ≥10 μM in microfluidic devices seeded with human liver cells. ALT, AST, and glutamate dehydrogenase (GLDH) were also elevated in dog and human microfluidic device liver systems, however, albumin was a more sensitive marker of hepatocyte dysfunction. Thus, the microfluidic device liver system data corroborate past in vivo results and suggest that the dog is more sensitive to some compound's toxicity than humans; however, as this is only a difference in potency, the liver toxicity observed in dogs would have likely translated to humans depending on clinical dose.

VII. Identifying Risk for Idiosyncratic DILI Using Microfluidic Devices Seeded with Liver Cells

One of the most difficult forms of hepatotoxicity relates to idiosyncratic DILI responses that are often missed during preclinical testing. To explore whether microfluidic devices seeded with human liver cells might be useful to predict these types of response, TAK-875, a GPR40 agonist that was discontinued in Phase III trials due to low incidence (2.7%) treatment-related elevations in transaminases (>3-fold rise in upper limit of normal) combined with a few individual cases of serious DILI was tested. In vitro and in vivo studies identified formation of reactive acyl glucuronide metabolites, suppression of mitochondrial respiration, and inhibition of hepatic transporters by TAK-875 as potential mediators of its hepatotoxic effects.

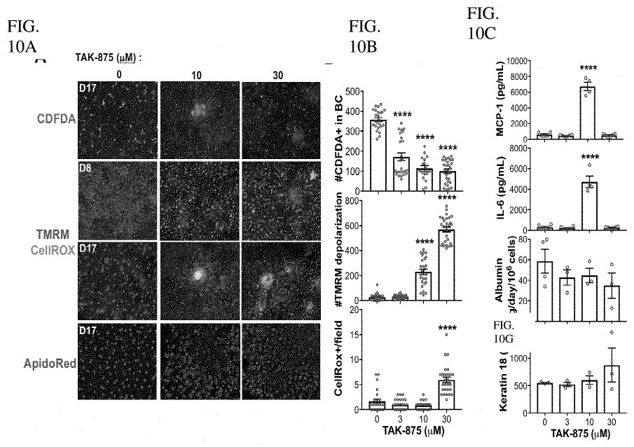

Intracellular accumulation of TAK-875AG in microfluidic devices seeded with liver cells is likely a consequence of its effect on MRP transporters because glucuronide metabolites are substrates for canalicular and basolateral hepatic MRP transporters, but at high concentrations they may inhibit their own efflux and accumulate in hepatocytes. This was confirmed in microfluidic devices seeded with liver cells, as indicated by a dose-dependent decrease in biliary efflux of the MRP2 substrate CDFDA, suggestive of competitive inhibition by TAK-875AG (FIG. 10A). This allowed the probing of the consequences of prolonged exposures to TAK-875 and its reactive acyl glucuronide metabolite in microfluidic devices seeded with liver cells which was found to have an effect on mitochondrial membrane potential, as indicated by a dose-related and time-dependent redistribution of the mitochondrial potential sensitive dye tetramethylrhodamine methyl ester (TMRM) following treatment (FIG. 10A and FIG. 10B). Lipid droplet accumulation was also detected at the end of the 2-week treatment, which is a physiological consequence of perturbation of the mitochondria (FIG. 10A). These effects were accompanied by an increase in formation of ROS, which is an expected consequence of inhibiting mitochondrial complex-1 (FIG. 10A and FIG. 10B).

It was then investigated whether an innate response could be detected in microfluidic devices seeded with liver cells treated with TAK-875 based on the prevailing hypothesis for DILI, which is that haptenization by reactive metabolites combined with cell stress can cause release of damage associated molecular patterns (DAMPs) and initiate an innate response followed by an adaptive immune attack of hepatocytes. As expected, treatment with TAK-875 caused significant release of the inflammatory cytokines MCP-1 and IL-6 at 10 μM; this was not observed at the highest dose (30 μM), likely because cell injury was observed at this concentration, as measured by decreased albumin production and increased release of K18 (FIG. 10C). Although steatosis was noted in liver biopsies in patients administered TAK-875, it was challenging to assign causality because of disease background in Type 2 diabetic patients; however, these findings suggest that microvesicular steatosis secondary to mitochondrial dysfunction could be a phenotype of DILI following treatment with TAK-875. The mitochondrial perturbations caused by TAK-875 in microfluidic devices seeded with human liver cells were only accompanied by minimal changes in markers of hepatocyte function or necrosis (albumin and K18; FIG. 10C), suggesting that use of these endpoints alone would miss the underlying mechanisms identified in the microfluidic device liver system.

FIGS. 10A-10C depicts how to identify risk for idiosyncratic DILI using microfluidic devices seeded with human liver cells. FIG. 10A shows representative images of CDFDA (green, DAPI in blue) to identify MRP2 transporter activity. TMRM and CellROX (red and cyan respectively, DAPI in blue) to detect mitochondrial depolarization and ROS respectively, and AdipoRed (red, DAPI in blue) to detect lipid droplets after daily administration of TAK-875 at 10 and 30 μM for 8 days or 15 days in microfluidic devices seeded with human liver cells. FIG. 10B shows quantifications of number of CDFDA positive fractions in bile canaliculi area, number of redistributed TMRM fractions and CellROX positive events per field of view after daily administration of TAK-875 at 3, 10, and 30 μM for 15 days in microfluidic devices seeded with human liver cells. Kruskal-Wallis tests (n=3 independent microfluidic devices with 5 randomly selected different areas per microfluidic device). FIG. 10C shows MCP-1 and IL-6 releases from the vascular channel and albumin and keratin 18 secretions from the parenchymal channel after 14 days of TAK-875 treatment in microfluidic devices seeded with human liver cells. Dunnett's multiple comparisons test (n=3 independent microfluidic devices). ****P<0.0001. Error bars present mean±SEM.

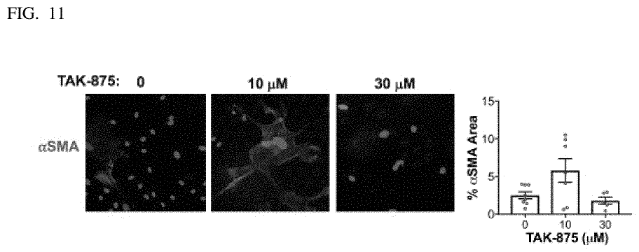

FIG. 11 shows stellate cell activation following TAK-875 treatment. Representative images of aSMA (red, DAPI in blue) to detect activated stellate cells after daily administration of TAK-875 at 10 or 30 uM for 15 days in human Liver-Chips. Quantifications of % aSMA positive area from the vascular channel. Not significant (n=2 independent chips with 3-5 randomly selected different areas per chip).

[symbol]

increased albumin secretion and CYP activities) in short-term (e.g., 1 day) cultures. In contrast, in the present study, it has been showed that both the dual-cell and quadruple-cell microfluidic device liver systems remain metabolically competent and maintain albumin production as well as activities of multiple drug metabolizing enzymes at in vivo-like levels for at least 14 days in culture. While DILI responses to various drugs were measured in other MPS and microfluidic liver models, the drug concentrations utilized were not clinically relevant. In contrast, drug levels similar to those observed in the plasma of animals and patients in the present study were used, and as a result, results that closely mimic those previously reported in both preclinical animal studies and human clinical trials were able to be generated.

Endpoints assessed in most in vitro systems are limited to measures of cell viability as an initial assessment of potential hazards, but they often do not capture the mechanisms that underlie DILI and they are not effective for human risk assessment. It was evaluated whether the microfluidic device liver system could detect more complex and mechanistically relevant DILI endpoints. Using a combination of microscopy, tissue staining, and measurement of DILI biomarkers, diverse phenotypes of DLI including hepatocellular injury, cholestasis, steatosis, Kupffer cell depletion, and stellate cell activation as a marker of fibrosis were able to be detected. Thus, the quadruple-cell microfluidic device liver system appears to be suitable for detecting toxicities that are attributable to direct effects on the four liver cell types included in our model; however, they are not capable of detecting toxicities of the bile duct. It was interesting to note that, with the tool compounds tested so far, toxicities in the model were detected at concentrations that bridged human plasma levels associated with DILI, suggesting that the model has potential to be used for human risk assessment.

The ability to measure mechanistic endpoints and biomarkers in the model also makes it suitable for delineating pathways and mechanisms causing DILI. For instance, the observation that GSH and ATP depletion are early events in APAP-mediated toxicity followed by a decline in hepatocyte function and finally by oxidative stress and overt injury, suggests that toxic metabolite-mediated mitochondrial dysfunction and ATP depletion are likely early events in the APAP toxicity cascade. Indeed, mitochondrial dysfunction has been identified as a hazard for APAP toxicity. Depletion of GSH and ATP in non-parenchymal cells following treatment with APAP implies that the toxic metabolite can escape hepatocytes and mediate an effect on other cell types, or that these cells have intrinsic metabolic activity. Low mRNA levels of CYP1A1 and CYP2B were detected in the non-parenchymal layer in microfluidic devices seeded with liver cells, and similar low levels of CYP activity have been previously reported in Stellate cells. These studies also confirm that both hepatocytes and non-parenchymal contribute to APAP hepatoxicity.

Specific contexts of use may be defined for predictive in vitro models, such as the microfluidic device liver system, prior to their qualification to make decisions in the drug development process. These contexts of use can include prediction of human liver toxicity, human relevance of toxicity observed in animal studies, or identifying DILI potential of compounds that form reactive metabolites. Results of our studies with bosentan, fialuridine, methotrexate, and a JNJ compound show that the microfluidic devices seeded with human liver cells can be used to predict diverse liver toxicities. Observed liver toxicities occur at concentrations that bridge plasma concentrations where the toxicities were observed in humans. Human-specific sensitivities to toxicity by bosentan and FIAU were also confirmed in the microfluidic devices seeded with human liver cells compared to the companion microfluidic devices seeded with animal liver cells. The putative mechanism for bosentan—inhibition of bile acid efflux via BSEP resulting in intracellular accumulation of bile acids—was also confirmed; however, an advantage of the microfluidic device liver system is that these mechanistic endpoints could be coupled to a measurable decline in hepatocyte function. The species differences in bosentan toxicity may be related to the presence of factors that mitigate the impact of BSEP inhibition in the rat, but not in humans. Alternatively, rats may be less susceptible to bosentan-induced hepatotoxicity because rat bile acids are inherently less toxic than human bile acids, and bosentan inhibits Na+-dependent taurocholate uptake in rat hepatocytes. The species differences for FIAU can be explained by lack of expression and activity of the nucleoside transporter I (EMTI), which facilitates entry of FIAU into the mitochondrial membrane in rodents compared to human EMT1 which initiates mitochondrial toxicity.

A gap exists in assigning human relevance of liver toxicities observed in animal studies, especially when these are observed in only one species. At least two JNJ compounds tested, are examples of compounds that caused liver toxicity in animal studies and were discontinued prior to clinical development because of lack of biomarkers to monitor for fibrosis in humans or severe liver toxicity in dogs. The microfluidic devices seeded with rat liver cells were very sensitive to treatment with one compound at early times, while no toxicity was observed in the microfluidic devices seeded with human liver cells at same concentrations up to 14 days of daily treatment. Activation of stellate cells noted in rat. but not the microfluidic devices seeded with human liver cells, confirmed that the pathophysiology observed in living rats could not be reproduced in the microfluidic devices seeded with rat liver cells in vitro. Moreover, the lack of a similar response in the microfluidic devices seeded with human liver cells suggests that this finding may not translate to humans. Although these results are interesting and could have influenced an internal decision to test the compound in non-rodents to address whether fibrosis was rat-specific, the model would need robust qualification with a specific context of use to convince regulatory agencies to make a decision with regards to the lack of human relevance of the rat findings. A release of cytokines/chemokines was observed including IP-10 in microfluidic device liver systems treated with MTX, FIAU, and a JNJ compound which has been identified as a potential marker for fibrosis, this model also may be amenable to biomarker discovery, especially for more challenging disease areas, such as steatosis and fibrosis, where suitable biomarkers for monitoring in humans are lacking.

Reactive metabolite formation has been identified as a hazard associated with compounds that cause rare or idiosyncratic DILI. Assays used to assess formation of reactive metabolites are often conducted in microsomes, hepatocyte suspensions, or in static plate cultures. The microfluidic device liver systems provide an opportunity to put formation of reactive metabolites in a cell, tissue and organ context for a functional readout of their contribution to DILI. For instance, studies conducted with TAK-875 show that continuous exposure to parent and reactive metabolites caused mitochondrial dysfunction, oxidative stress to cells, formation of lipid droplets, and an innate immune response (i.e., cytokine release). Thus, our results suggest that microfluidic devices seeded with human liver cells may enable a specific context of use to be developed for assessing causality of reactive metabolite formation and DILI in humans.

Although the enzymatic activities in the microfluidic devices seeded with human liver cells were robust, some activities were lower or higher in comparison to fresh human hepatocytes, which may reflect donor to donor variability. The CYP enzyme activities in microfluidic devices seeded with rat liver cells also were similar to those measured in fresh rat hepatocytes, when a single substrate was used, but they were higher when a cocktail of CYP substrates were used, which suggests that there might be interactions among these substrates (FIG. 14 and FIG. 15B).

Also, it has been reported that octamethylcyclotetrasiloxane (D4), which is an intermediate in the synthesis of poly-dimethylsiloxane (PDMS) that is used to fabricate the microfluidic devices, can induce rat CYP2B1/2; so it is possible that release of low levels of this chemical from the body of the microfluidic devices could potentially induce this gene in our model. Compounds that undergo turnover by rat CYP2B1/2, or those that induce these enzymes, should therefore be evaluated carefully to avoid incorrect interpretations when using these microfluidic devices; however, the general impact on compound risk assessment is low given that multiple CYP enzymes are commonly analyzed in parallel.

One advantage of the microfluidic device liver systems is the use of continuous flow in an open system, which ensures that cells are exposed to sufficient levels of the parent drug and its metabolites simply by adjusting the flow rate. The open system also allows for continuous collection or sampling of the effluents of both the vascular and parenchymal channels, which prevents accumulation of the parent and metabolites, while enabling measurement of biomarkers and other biology endpoints over time. Metabolite identification studies with TAK-875 confirmed that the microfluidic devices seeded with human liver cells generates metabolites at relative amounts that are similar to those reported in humans.

Also, while some compounds have been reported to adsorb to the PDMS material used in the microfluidic devices, there has been acceptable recovery following administration of compounds to the microfluidic devices in the absence or presence of adherent cells.

In conclusion, it has been shown that species-specific microfluidic device liver systems have potential future application for safety testing, disease modeling, and predicting human hepatotoxicities, including idiosyncratic responses. This approach also could be used to query human relevance of toxicities observed in preclinical animal studies or for mechanistic investigations of DILI detected in nonclinical and clinical studies.

### Exemplary Chip Activation

A. Chip Activation (Functionalization) Compounds

In one embodiment, bifunctional crosslinkers are used to attach one or more extracellular matrix (ECM) proteins. A variety of such crosslinkers are available commercially, including (but not limited to) the following compounds:

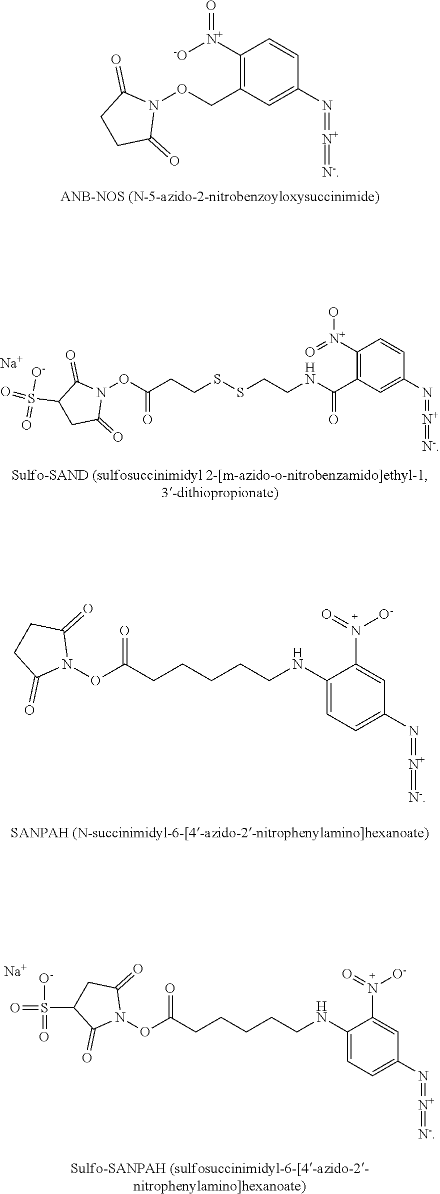

By way of example, sulfosuccinimidyl 6-(4′-azido-2′-nitrophenyl-amino) hexanoate or “Sulfo-SANPAH” (commercially available from Pierce) is a long-arm (18.2 angstrom) crosslinker that contains an amine-reactive N-hydroxysuccinimide (NHS) ester and a photoactivatable nitrophenyl azide. NHS esters react efficiently with primary amino groups (—NH₁) in pH 7-9 buffers to form stable amide bonds. The reaction results in the release of N-hydroxy-succinimide. When exposed to UV light, nitrophenyl azides form a nitrene group that can initiate addition reactions with double bonds, insertion into C—H and N—H sites, or subsequent ring expansion to react with a nucleophile (e.g., primary amines). The latter reaction path dominates when primary amines are present.

Sulfo-SANPAH should be used with non-amine-containing buffers at pH 7-9 such as 20 mM sodium phosphate, 0.15 M NaCl; 20 mM HEPES; 100 mM carbonate/bicarbonate; or 50 mM borate. Tris, glycine or sulfhydryl-containing buffers should not be used. Tris and glycine will compete with the intended reaction and thiols can reduce the azido group.

For photolysis, one should use a UV lamp that irradiates at 300-460 nm. High wattage lamps are more effective and require shorter exposure times than low wattage lamps. UV lamps that emit light at 254 nm should be avoided; this wavelength causes proteins to photodestruct. Filters that remove light at wavelengths below 300 nm are ideal. Using a second filter that removes wavelengths above 370 nm could be beneficial but is not essential.

B. Exemplary Methods of Chip Activation.
1. Prepare and sanitize hood working space
2. S-1 Chip (Tall Channel) Handling—Use aseptic technique, hold Chip using Carrier
   a. Use 70% ethanol spray and wipe the exterior of Chip package prior to bringing into hood
   b. Open package inside hood
   c. Remove Chip and place in sterile Petri dish (6 Chips/Dish)
   d. Label Chips and Dish with respective condition and Lot #
3. Surface Activation with Chip Activation Compound (light and time sensitive)
   a. Turn off light in biosafety hood
   b. Allow vial of Chip Activation Compound powder to fully equilibrate to ambient temperature (to prevent condensation inside the storage container, as reagent is moisture sensitive)
   c. Reconstitute the Chip Activation Compound powder with ER-2 solution
      i. Add 10 ml Buffer, such as HEPES, into a 15ml conical covered with foil
      ii. Take 1 ml Buffer from above conical and add to chip Activation Compound (5mg) bottle, pipette up and down to mix thoroughly and transfer to same conical
      iii. Repeat 3-5 times until chip Activation Compound is fully mixed
      iv. NOTE: Chip Activation Compound is single use only, discard immediately after finishing Chip activation, solution cannot be reused
   d. Wash channels
      i. Inject 200 ul of 70% ethanol into each channel and aspirate to remove all fluid from both channels
      ii. Inject 200 ul of Cell Culture Grade Water into each channel and aspirate to remove all fluid from both channels
      iii. Inject 200 ul of Buffer into each channel and aspirate to remove fluid from both channels
   e. Inject Chip Activation Compound Solution (in buffer) in both channels
      i. Use a P200 and pipette 200 ul to inject Chip Activation Compound/Buffer into each channel of each chip (200 ul should fill about 3 Chips (Both Channels))
      ii. Inspect channels by eye to be sure no bubbles are present. If bubbles are present, flush channel with Chip Activation Compound/Buffer until bubbles have been removed
   f. UV light activation of Chip Activation Compound Place Chips into UV light box
      i. UV light treat Chips for 20 min
      ii. While the Chips are being treated, prepare ECM Solution.
      iii. After UV treatment, gently aspirate Chip Activation Compound/Buffer from channels via same ports until channels are free of solution
      iv. Carefully wash with 200 ul of Buffer solution through both channels and aspirate to remove all fluid from both channels
      v. Carefully wash with 200 ul of sterile DPBS through both channels
      vi. Carefully aspirate PBS from channels and move on to: ECM-to-Chip
         Exemplary ECM-to-Chip: Coat Chips with ECM

In some preferred embodiments, liver chip channels are coated with ECM, e.g. Collagen I and Fibronectin; organ-specific extracellular matrix proteins; cell-specific extracellular matrix proteins; Matrigel® (BD Corning); etc. In some embodiments, ECM is a mixture of collagen I and fibronectin proteins. In some embodiments, both channels are coated with a mixture of Collagen I and Fibronectin. ECM material may be diluted in Dulbecco's phosphate-buffered saline (DPBS) (without Ca²⁺, Mg²⁺).

In some embodiments, liver chips were treated by perfusing with a compound. In some embodiments, liver chips undergo physiological flow rates. In some embodiments, both liver chip channels were perfused at compound-specific flow rates to provide a continuous supply of fresh dosing solution in media for the duration of the experiments. More specifically, merely for examples, bosentan, JNJ compounds, MTX and FIAU were perfused at 30 μL/hr, whereas APAP and TAK-875 were perfused at 10 μL/hr.

In some embodiments, an airway microfluidic device may be used for high content imaging, e.g. for cilia and other airway cell proteins. FIG. 24 shows an exemplary human Airway chip. Schematic diagram of one embodiment of a human Airway chip with a 3 um pore (e.g., PET) membrane in between airway epithelium and microvascular endothelium (left). Differentiated airway epithelium exhibits continuous tight junctional connections on-chip (e.g., Zo-1+ network of cells). Well-differentiated human airway epithelium generated on-chip contains goblet cells (MUCSAC+ cells) and demonstrates extensive coverage of ciliated cells labeled for alpha-tubulin (green). Nuclei are stained and colored blue. Scale bar, 20 urn.

### Open Top Chips.

Embodiments Of Open-Top Chip Incorporates Mechanical Stretching And Vascular Fluid Flow. The open top device fits into the adapter shown in FIG. 2. FIG. 25A shows a schematic of one embodiment of an assembled open -top chip microfluidic device 1700, showing open-top chambers 1763 and 1764 each located above a circular lower fluidic channel, e.g. 1751. Each chamber is surrounded by a deformable surface 1745 (e.g. membrane); spiral microchannels 1751 each are in fluidic communication with an inlet port 1719 located adjacent to an outlet port and an outlet port 1722 adjacent to an inlet port. Optionally a first vacuum port 1730; optionally a second vacuum port 1732, each vacuum port 1730 and 1732 connected to a first vacuum chamber 1737 or a second vacuum chamber 1738. FIG. 25B shows a schematic of one embodiment of an assembled open -top chip microfluidic device 1700, showing open-top chambers 1763 and 1764 each located above a circular lower fluidic channel, e.g. 1751. Each chamber is surrounded by a deformable surface 1745 (e.g. membrane); spiral microchannels 1751 each are in fluidic communication with an inlet port 1719 located adjacent to an outlet port and an outlet port 1722 adjacent to an inlet port. Optionally a first vacuum port 1730; optionally a second vacuum port 1732, each vacuum port 1730 and 1732 connected to a first vacuum chamber 1737 or a second vacuum chamber 1738.

FIG. 25B shows a schematic of one embodiment of an exploded view of the embodiment depicted FIG. 25A shows an open-top chip device 1800, wherein a membrane 1840 resides between the bottom surface of the first chamber 1863 and the second chamber 1864 and spiral microchannels 1851.

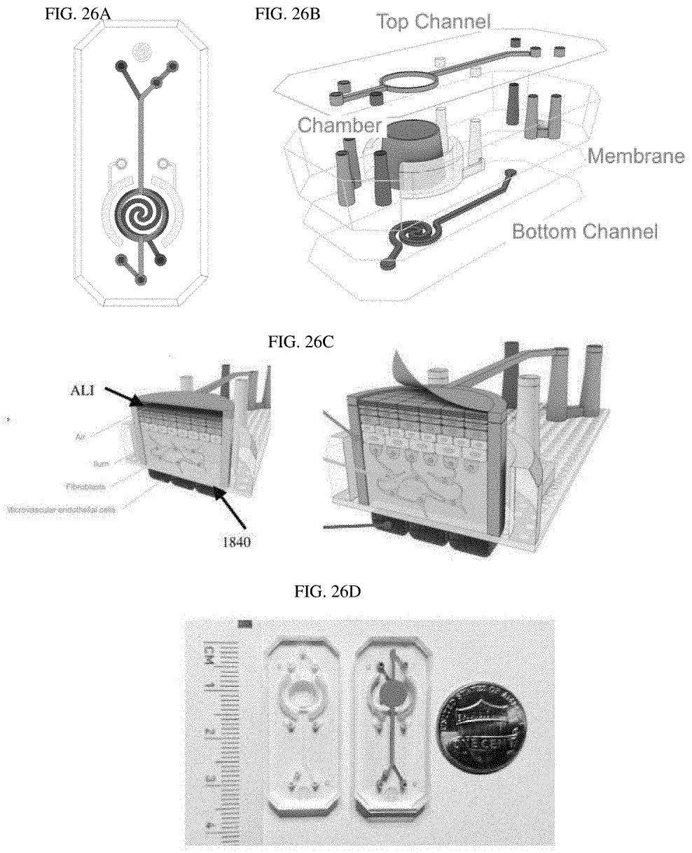

FIG. 26A shows a schematic of one-embodiment (top view) of chip 1800 with a single chamber showing one embodiment of lower channel 1851 (left) and a combined view of an upper (blue) and lower channel (red). Black dots represent inlet and outlet ports.

FIG. 26B Illustrates an exploded (layer by layer) view of one-embodiment of an open top device as shown in FIG. 25A, showing membrane 1840 in between a chamber (blue) and the bottom channel (red).

FIG. 26C shows an exemplary schematic of one embodiment of a 3D Alveolus Lung On-Chip as an open top microfluidic chip demonstrating an air layer on top of an epithelial layer, e.g., alveolar epithelium layer or airway cell layer, overlaying a stromal area, e.g., including fibroblast cells, in an upper chamber/channel with microvascular endothelial cells, as one example of endothelial cells, in a lower channel, e.g. showing a cut away view of multiple areas (rectangles) as part of one spiral channel (red). Left: showing location of air-liquid interface (ALI) and membrane 1840 with a top closed on an open top chip. Right: showing chamber walls—blue; growth chamber—yellow and vascular circular channel cut-put views—red with a top partially opened.

FIG. 26D shows a photograph of one embodiment of an actual open top chip, cm scale on the left, actual chip in the middle with one view showing an overlay of an upper channel (blue) and lower channel (red), with respect to a US Penny for size.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in biological control, biochemistry, molecular biology, or related fields are intended to be within the scope of the following claims.

1. A method of imaging microfluidic devices comprising:
(a) providing a microfluidic device comprising a porous membrane, said porous membrane separating a first microfluidic channel having an endothelial cell layer and a second microfluidic channel having a second cell layer;
(b) providing a microscope capable of image acquisition;
(c) taking a first set of microscopic image acquisitions;
(d) determining a focal height and locating a standard coordinate system from said first set of microscopic image acquisitions, wherein the coordinate system is located based on the location of the membrane within the microfluidic device; and
(e) taking a second set of microscopic image acquisitions based on the coordinate system located in the first set of microscopic acquisitions;
wherein said second set of microscopic image acquisitions comprise said endothelial cell layer and said second cell layer together, separated by the membrane.

2. The method of claim 1, wherein the microscope is a confocal microscope.

3. The method of claim 1, wherein the first set of microscopic acquisitions are low-resolution.

4. The method of claim 1, wherein the second set of microscopic acquisitions are high-resolution.

5. (canceled)

6. The method of claim 4, wherein the second set of microscope acquisitions are used to evaluate the effect of an agent on the cells.

7. The method of claim 6, wherein the agent is a pharmaceutical.

8. The method of claim 1, wherein the cells are cultured for more than seven days.

9. (canceled)

10. The method of claim 4, wherein the second set of microscopic acquisitions comprises a three-dimensional acquisition.

11. (canceled)

12. The method of claim 1, wherein the second cell layer comprises liver cells.

13. The method of claim 12, wherein the liver cells are hepatocytes.

14. The method of claim 13, wherein the hepatocytes are human hepatocytes.

15. The method of claim 1, wherein the second cell layer comprises kidney cells.

16. (canceled)

17. (canceled)

18. The method of claim 1, further comprising applying flow to the channels.

19. The method of claim 1, wherein the second set of acquisitions, guided by the coordinate system, comprises Z stack slices through different layers of the microfluidic device.

20-37. (canceled)

38. A method of imaging microfluidic devices comprising:
(a) providing a microfluidic device comprising a membrane having pores, said membrane separating two microfluidic channels;
(b) providing a microscope capable of image acquisition;
(c) taking a set of low resolution microscopic image acquisitions;
(d) locating a standard coordinate system using said set of low resolution image acquisitions, wherein the coordinate system is located based on the location of said pores; and
(e) taking a set of high resolution microscopic acquisitions based on the coordinate system located in the first set of microscopic acquisitions.

39-61. (canceled)

62. The method of claim 38, wherein the microfluidic device is seeded with cells.

63. The method of claim 62, wherein the high resolution set of microscopic image acquisitions is used to evaluate the effect of an agent on the cells.

64-68. (canceled)

69. The method of claim 62, wherein the cells are liver cells.

70. The method of claim 69, wherein the liver cells are hepatocytes and sinusoidal endothelial cells.

71. (canceled)

72. The method of claim 62, wherein the cells are kidney cells.

73. The method of claim 62, wherein the microscopic acquisitions are of individual cells.

74. (canceled)

75. The method of claim 62, further comprising applying flow to the channels.

76. The method of claim 75, where in the flow exerts shear stress on the cells.

77. A method of analyzing cellular phenotype changes following agent exposure comprising:
(a) providing a plurality of microfluidic devices comprising cells in microchannels, said microchannels comprising microchannel walls;
(b) providing a microscope capable of image acquisition;
(c) treating a number of said microfluidic devices with an agent and a number of said microfluidic devices with a control media;
(d) taking a first set of microscopic acquisitions;
(e) locating a standard coordinate system using the first set of microscope acquisitions, wherein the coordinate system is located based on the location of the microchannel walls within the microfluidic device;
(f) taking a second set of microscopic acquisitions based on the coordinate system located in the first set of microscopic acquisitions;
(g) making endpoint measurements of the acquisitions;
(h) fitting a regression model to the measurements;
(i) estimating a field effect based on the regression; and
(j) comparing the field effect from microfluidic devices treated with an agent verses microfluidic device treated with a control media;
wherein said high resolution microscopic acquisition comprises a three-dimensional microscopic acquisition.

78. The method of claim 77, wherein said regression model is a Bayesian linear regression model.

79. The method of claim 77, wherein said field effect is a linear field effect.

80. The method of claim 77, wherein the microscope is a confocal microscope.

81. The method of claim 77, wherein the first set of microscopic acquisitions are low-resolution.

82. The method of claim 77, wherein the second set of microscopic acquisitions are high-resolution.

83. The method of claim 77, wherein the agent is a pharmaceutical.

84-85. (canceled)

86. The method of claim 77, wherein the three-dimensional acquisition comprises an endothelial cell layer and hepatocyte cell layer together, separated by the membrane.

87. The method of claim 77, wherein the cells are liver cells.

88. The method of claim 87, wherein the liver cells are hepatocytes and sinusoidal endothelial cells.

89. The method of claim 88, wherein the hepatocytes and sinusoidal endothelial cells are human hepatocytes and human sinusoidal endothelial cells.

90. The method of claim 77, wherein the cells are kidney cells.

91. The method of claim 77, wherein the microscopic acquisitions are of individual cells.

92. The method of claim 77, further comprising applying flow to the channels.

98-110. (canceled)
